SEQUENCE LISTING (79 sequences)

SEQ ID NO: 1 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 743 aa, Artificial Sequence
  X1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 743 aa, Artificial Sequence
  X1.1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIDGAATKNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 743 aa, Artificial Sequence
  X1.2
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIDGQSSKSQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 743 aa, Artificial Sequence
  X1.3
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTILQTSSPGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 743 aa, Artificial Sequence
  X1.4
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 743 aa, Artificial Sequence
  X1.5
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 743 aa, Artificial Sequence
  X1.6
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGAQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 743 aa, Artificial Sequence
  AAV1-X1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSGNNTRSVTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - protein, 743 aa, Artificial Sequence
  X2
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNNTRDTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 743 aa, Artificial Sequence
  X3
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTNSTRPVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 7 aa, Artificial Sequence
  X1-7mer
GNNTRSV

SEQ ID NO: 14 - protein, 7 aa, Artificial Sequence
  X2-7mer
GNNTRDT

SEQ ID NO: 15 - protein, 7 aa, Artificial Sequence
  X3-7mer
TNSTRPV

SEQ ID NO: 16 - protein, 7 aa, Artificial Sequence
  X1.1-7mer
DGAATKN

SEQ ID NO: 17 - protein, 7 aa, Artificial Sequence
  X1.2-7mer
DGQSSKS

SEQ ID NO: 18 - protein, 7 aa, Artificial Sequence
  X1.3-7mer
LQTSSPG

SEQ ID NO: 19 - protein, 14 aa, Artificial Sequence
  AAV9 588_589
TNHQSAQAQAQTGW

SEQ ID NO: 20 - protein, 14 aa, Artificial Sequence
  AAV1 588_589
VNFQSSSTDPATGD

SEQ ID NO: 21 - protein, 21 aa, Artificial Sequence
  PHP.B 588_589
TNHQSAQTLAVPFKAQAQTGW

SEQ ID NO: 22 - protein, 21 aa, Artificial Sequence
  AAV1-PHP.B 588_589
VNFQSSSTLAVPFKTDPATGD

SEQ ID NO: 23 - protein, 21 aa, Artificial Sequence
  X1 588_589
TNHQSAQGNNTRSVAQAQTGW

SEQ ID NO: 24 - protein, 21 aa, Artificial Sequence
  AAV1-X1 588_589
VNFQSSSGNNTRSVTDPATGD

SEQ ID NO: 25 - DNA, 24 bp, Artificial Sequence
  FXN barcode F primer
tggacctaagcgttatgactggac

SEQ ID NO: 26 - DNA, 31 bp, Artificial Sequence
  FXN barcode R primer
ggagcaacatagttaagaataccagtcaatc

SEQ ID NO: 27 - DNA, 35 bp, Artificial Sequence
  Adaptor F
acgctcttccgatcttgttccagattacgcttgag

SEQ ID NO: 28 - DNA, 39 bp, Artificial Sequence
  Adaptor R
tgtgctcttccgatcttgtaatccagaggttgattatcg

SEQ ID NO: 29 - protein, 4 aa, Artificial Sequence
  X1-4mer1
GNNT

SEQ ID NO: 30 - protein, 4 aa, Artificial Sequence
  X1-4mer2
NNTR

SEQ ID NO: 31 - protein, 4 aa, Artificial Sequence
  X1-4mer3
NTRS

SEQ ID NO: 32 - protein, 4 aa, Artificial Sequence
  X1-4mer4
TRSV

SEQ ID NO: 33 - protein, 5 aa, Artificial Sequence
  X1-5mer2
GNNTR

SEQ ID NO: 34 - protein, 5 aa, Artificial Sequence
  X1-5mer2
NNTRS

SEQ ID NO: 35 - protein, 5 aa, Artificial Sequence
  X1-5mer3
NTRSV

SEQ ID NO: 36 - protein, 6 aa, Artificial Sequence
  X1-6mer1
GNNTRS

SEQ ID NO: 37 - protein, 6 aa, Artificial Sequence
  X1-6mer2
NNTRSV

SEQ ID NO: 38 - protein, 4 aa, Artificial Sequence
  X2-4mer2
NTRD

SEQ ID NO: 39 - protein, 4 aa, Artificial Sequence
  X2-4mer2
TRDT

SEQ ID NO: 40 - protein, 5 aa, Artificial Sequence
  X2-5mer1
NNTRD

SEQ ID NO: 41 - protein, 5 aa, Artificial Sequence
  X2-5mer2
NTRDT

SEQ ID NO: 42 - protein, 6 aa, Artificial Sequence
  X2-6mer1
GNNTRD

SEQ ID NO: 43 - protein, 6 aa, Artificial Sequence
  X2-6mer2
NNTRDT

SEQ ID NO: 44 - protein, 4 aa, Artificial Sequence
  X3-4mer1
TNST

SEQ ID NO: 45 - protein, 4 aa, Artificial Sequence
  X3-4mer2
NSTR

SEQ ID NO: 46 - protein, 4 aa, Artificial Sequence
  X3-4mer3
STRP

SEQ ID NO: 47 - protein, 4 aa, Artificial Sequence
  X3-4mer4
TRPV

SEQ ID NO: 48 - protein, 5 aa, Artificial Sequence
  X3-5mer1
TNSTR

SEQ ID NO: 49 - protein, 5 aa, Artificial Sequence
  X3-5mer2
NSTRP

SEQ ID NO: 50 - protein, 5 aa, Artificial Sequence
  X3-5mer3
STRPV

SEQ ID NO: 51 - protein, 6 aa, Artificial Sequence
  X3-6mer1
TNSTRP

SEQ ID NO: 52 - protein, 6 aa, Artificial Sequence
  X3-6mer2
NSTRPV

SEQ ID NO: 53 - protein, 4 aa, Artificial Sequence
  X1.1-4mer1
DGAA

SEQ ID NO: 54 - protein, 4 aa, Artificial Sequence
  X1.1-4mer2
GAAT

SEQ ID NO: 55 - protein, 4 aa, Artificial Sequence
  X1.1-4mer3
AATK

SEQ ID NO: 56 - protein, 4 aa, Artificial Sequence
  X1.1-4mer4
ATKN

SEQ ID NO: 57 - protein, 5 aa, Artificial Sequence
  X1.1-5mer
DGAAT

SEQ ID NO: 58 - protein, 5 aa, Artificial Sequence
  X1.1-5mer2
GAATK

SEQ ID NO: 59 - protein, 5 aa, Artificial Sequence
  X1.1-5mer3
AATKN

SEQ ID NO: 60 - protein, 6 aa, Artificial Sequence
  X1.1-6mer1
DGAATK

SEQ ID NO: 61 - protein, 6 aa, Artificial Sequence
  X1.1-6mer2
GAATKN

SEQ ID NO: 62 - protein, 4 aa, Artificial Sequence
  X1.2-4mer
DGQS

SEQ ID NO: 63 - protein, 4 aa, Artificial Sequence
  X1.2-4mer2
GQSS

SEQ ID NO: 64 - protein, 4 aa, Artificial Sequence
  X1.2-4mer3
QSSK

SEQ ID NO: 65 - protein, 4 aa, Artificial Sequence
  X1.2-4mer4
SSKS

SEQ ID NO: 66 - protein, 5 aa, Artificial Sequence
  X1.2-5mer
DGQSS

SEQ ID NO: 67 - protein, 5 aa, Artificial Sequence
  X1.2-5mer2
GQSSK

SEQ ID NO: 68 - protein, 5 aa, Artificial Sequence
  X1.2-5mer3
QSSKS

SEQ ID NO: 69 - protein, 6 aa, Artificial Sequence
  X1.2-6mer
DGQSSK

SEQ ID NO: 70 - protein, 6 aa, Artificial Sequence
  X1.2-6mer2
GQSSKS

SEQ ID NO: 71 - protein, 4 aa, Artificial Sequence
  X1.3-4mer
LQTS

SEQ ID NO: 72 - protein, 4 aa, Artificial Sequence
  X1.3-4mer2
QTSS

SEQ ID NO: 73 - protein, 4 aa, Artificial Sequence
  X1.3-4mer3
TSSP

SEQ ID NO: 74 - protein, 4 aa, Artificial Sequence
  X1.3-4mer4
SSPG

SEQ ID NO: 75 - protein, 5 aa, Artificial Sequence
  X1.3-5mer
LQTSS

SEQ ID NO: 76 - protein, 5 aa, Artificial Sequence
  X1.3-5mer2
QTSSP

SEQ ID NO: 77 - protein, 5 aa, Artificial Sequence
  X1.3-5mer3
TSSPG

SEQ ID NO: 78 - protein, 6 aa, Artificial Sequence
  X1.3-6mer
LQTSSP

SEQ ID NO: 79 - protein, 6 aa, Artificial Sequence
  X1.3-6mer2
QTSSPG

(19) United States
(12) Patent Application Publication
(10) Pub. No.: US 2024/0197919 A1
(43) Pub. Date: Jun. 20, 2024

(54) RECOMBINANT AAVS FOR DELIVERY TO CENTRAL NERVOUS SYSTEM AND BRAIN VASCULATURE

(71) Applicant: California Institute of Technology, Pasadena, CA (US)

(72) Inventors: Viviana Gradinaru, Pasadena, CA (US); Xinhong Chen, Pasadena, CA (US)

(21) Appl. No.: 18/288,910

(22) PCT Filed: May 3, 2022
(86) PCT No.: PCT/US2022/027516

§ 371 Date: Oct. 30, 2023

Related U.S. Application Data
(60) Provisional application No. 63/183,702, filed on May 4, 2021.

(51) Int. Cl. A61K 48/00; C07K 14/005; C07K 14/47; C12N 15/86

(52) U.S. Cl. CPC A61K 48/0058; C07K 14/005; C07K 14/47; C12N 15/86; C12N 2750/14122; C12N 2750/14143; C12N 2750/14145

(57) ABSTRACT

Disclosed herein include compositions and kits comprising recombinant adeno-associated viruses (rAAVs) with tropisms to the central nervous system with increased specificity and transduction efficiency, including endothelial cells of the neurovascular unit. Also described include methods of treating various diseases and conditions using the rAAVs.

### RELATED APPLICATIONS

This application claims the benefit under 35 U.S.C. § 119(e) of U.S. Provisional Patent Application Ser. No. 63/183,702, filed on May 4, 2021, the content of this related application is incorporated herein by reference in its entirety for all purposes.

### STATEMENT REGARDING FEDERALLY SPONSORED R&D

This invention was made with government support under Grant Nos. NS111369 & NS087949 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302444-WO_Sequence_Listing, created May 1, 2022, which is 87 kilobytes in size. The information in the electronic format of the Sequence Listing is incorporated herein by reference in its entirety.

### BACKGROUND

### Field

The present disclosure relates generally to the field of adeno-associated virus vectors. More specifically, the disclosure relates to central nervous system-tropic and endothelial-tropic AAVs for genetic access to whole-brain vasculature following non-invasive systemic delivery.

### Description of the Related Art

Vasculature is a vital component of the central nervous system, and malfunction of cell types comprising the brain vasculature, including endothelial cells, can facilitate the progression of neurological disorders. However, the study of vasculature is hampered by limited options for versatile, cell-type-specific transgene delivery. Adeno-associated virus (AAV) vectors offer promise for gene delivery to the brain, but are commonly administered via intracranial injections, resulting in tissue damage and limited and uneven spatial coverage. There is a need for systemic AAV delivery that provides a non-invasive, brain-wide alternative.

[symbol]

(BBB) with broad tropism in rodents (e.g., AAV-PHP.eB), but there is a need to identify vectors with cell-type biased tropism. From the PHP.B sequence family, PHP.V1 was identified, which, when intravenously delivered, has enhanced potency for endothelial cells although it also transduces astrocytes and neurons. While an improvement, PHP.V1 still requires cell type-specific promoters whose large size limits the choice of transgenes. In addition, capsid entry into other cell types may induce an immune response, creating a confounding effect.

Immune responses also limit AAV re-administration, which may be needed to maximize therapeutic effect, particularly given the loss of transgene expression over time observed with AAV gene delivery. While neutralizing antibodies induced by initial AAV administration can prevent sequential administration of the same AAV, switching to another AAV serotype with similar or complementary features is a potential solution that remains underexplored.

An AAV with potent and specific tropism for brain vasculature would enable new strategies for gene therapy. AAV vectors have been used to deliver diverse therapeutic genes to treat a broad spectrum of disorders, including those resulting from loss of either cell-autonomous factors or factors which act on neighboring cells regardless of genotype. For disorders caused by the loss of function of a single non-cell-autonomous factor, such as mucopolysaccharidosis, the factor is typically a secreted protein. In these applications, gene therapy targets a healthy cell population, transforming those cells into a ‘biofactory’ for production and secretion of a therapeutic protein that can cross-correct affected cells. Currently, however, therapeutic proteins produced from peripheral biofactories, most commonly the liver, enter the CNS with low efficiency and fall short of rescuing phenotypes in CNS disorders. Given its broad distribution and close proximity to other cell types within the CNS, AAV-transformed brain vasculature can serve as a better biofactory for the CNS. There is a need for AAV vectors comprising targeting peptides for efficiently transducing tissues of the CNS, e.g., endothelial tissues in the CNS.

### SUMMARY

Disclosed herein include adeno-associated virus (AAV) targeting peptides. In some embodiments, the targeting peptide comprises an amino acid sequence that comprises at least 4 contiguous amino acids from a sequence selected from the group consisting of GNNTRSV (SEQ ID NO: 13), GNNTRDT (SEQ ID NO: 14) and TNSTRPV (SEQ ID NO: 15).

In some embodiments, the targeting peptide comprises at least 5 contiguous amino acids from the sequence of GNNTRSV (SEQ ID NO: 13). In some embodiments, the (SEQ ID NO: 13). In some embodiments, the targeting peptide comprises GNNTRSV (SEQ ID NO: 13). In some embodiments, the targeting peptide comprises at least 4 contiguous amino acids from the sequence GNNTRDT (SEQ ID NO: 14). In some embodiments, the targeting peptide comprises at least 5 contiguous amino acids from the sequence of GNNTRDT (SEQ ID NO: 14). In some embodiments, the targeting peptide comprises at least 6 contiguous amino acids from the sequence of GNNTRDT (SEQ ID NO: 14). In some embodiments, the targeting peptide comprises GNNTRDT (SEQ ID NO: 14). In some embodiments, the targeting peptide comprises at least 4 contiguous amino acids from the sequence TNSTRPV (SEQ ID NO: 15). In some embodiments, the targeting peptide comprises at least 5 contiguous amino acids from the sequence of TNSTRPV (SEQ ID NO: 15). In some embodiments, the targeting peptide comprises at least 6 contiguous amino acids from the sequence of TNSTRPV (SEQ ID NO: 15). In some embodiments, the targeting peptide comprises TNSTRPV (SEQ ID NO: 15).

In some embodiments, the targeting AAV peptide is part of an AAV, for example part of a capsid protein of the AAV. In some embodiments, the targeting peptide is conjugated to a nanoparticle, a second molecule, a viral capsid protein, or a combination thereof. In some embodiments, the targeting peptide is a central nervous system (CNS) targeting peptide.

Disclosed herein include AAV capsid proteins. In some embodiments, the AAV capsid proteins comprise an AAV targeting peptide disclosed herein. The AAV capsid protein can comprise at least 4 contiguous amino acids from a second amino acid sequence selected from the group consisting of DGQSSKS (SEQ ID NO: 17), DGAATKN (SEQ ID NO: 16), and LQTSSPG (SEQ ID NO: 18). The AAV capsid protein can comprise at least 5 contiguous amino acids from a second amino acid sequence selected from the group consisting of DGQSSKS (SEQ ID NO: 17), DGAATKN (SEQ ID NO: 16), and LQTSSPG (SEQ ID NO: 18). The AAV capsid protein can comprise at least 6 contiguous amino acids from a second amino acid sequence selected from the group consisting of DGQSSKS (SEQ ID NO: 17), DGAATKN (SEQ ID NO: 16), and LQTSSPG (SEQ ID NO: 18). The AAV capsid protein can comprise a second amino acid sequence selected from the group consisting of DGQSSKS (SEQ ID NO: 17), DGAATKN (SEQ ID NO: 16), and LQTSSPG (SEQ ID NO: 18). In some embodiments, the at least 4, 5 or 6 contiguous amino acids from the second amino acid sequence replace at least 4, 5, 6 or 7 amino acids in AA452-458, or functional equivalents thereof, of the AAV capsid protein. In some embodiments, the at least 4, 5 or 6 contiguous amino acids from the second amino acid sequence, or the second amino acid sequence, replace at least 4, 5, 6 or 7 amino acids in the 455 loop, or functional equivalents thereof, of the AAV capsid protein. The AAV capsid protein can comprise one or more of amino acid substitutions at position N272, N272A, S386A, W503A, and W503R.

In some embodiments, the AAV capsid is derived from AAV9, or a variant thereof. In some embodiments, the AAV capsid is derived from an AAV selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10.

Disclosed herein include nucleic acids comprising a sequence encoding an AAV targeting peptide described herein. Disclosed herein include nucleic acids comprising a sequence encoding an AAV capsid protein described herein.

Disclosed herein include recombinant adeno-associated viruses (rAAV). In some embodiments, the rAAV comprises an AAV targeting peptide or AAV capsid protein described herein. In some embodiments, the rAAV comprises an AAV capsid protein. In some embodiments, the AAV capsid protein comprises an AAV targeting peptide described herein. In some embodiments, the amino acid sequence is inserted between two adjacent amino acids in AA586-592, or functional equivalents thereof, of the AAV capsid protein.

In some embodiments, the two adjacent amino acids are AA588 and AA589. In some embodiments, the AAV capsid protein comprises, or consists thereof, SEQ ID NOs: 1 or 2. The rAAV can comprise at least 4 contiguous amino acids from a second amino acid sequence selected from the group consisting of DGQSSKS (SEQ ID NO: 17), DGAATKN (SEQ ID NO: 16), and LQTSSPG (SEQ ID NO: 18). The rAAV can comprise at least 5 contiguous amino acids from a second amino acid sequence selected from the group consisting of DGQSSKS (SEQ ID NO: 17), DGAATKN (SEQ ID NO: 16), and LQTSSPG (SEQ ID NO: 18). The rAAV can comprise at least 6 contiguous amino acids from a second amino acid sequence selected from the group consisting of DGQSSKS (SEQ ID NO: 17), DGAATKN (SEQ ID NO: 16), and LQTSSPG (SEQ ID NO: 18). The rAAV can comprise a second amino acid sequence selected from the group consisting of DGQSSKS (SEQ ID NO: 17), DGAATKN (SEQ ID NO: 16), and LQTSSPG (SEQ ID NO: 18). In some embodiments, the at least 4, 5, or 6 contiguous amino acids from a second amino acid sequence, or the second amino acid sequence, replace at least 4, 5, 6 or 7 amino acids in AA452-458, or functional equivalents thereof, of the AAV capsid protein. In some embodiments, the at least 4, 5, or 6 contiguous amino acids from a second amino acid sequence, or the second amino acid sequence, replace at least 4, 5, 6 or 7 amino acids in the 455 loop, or functional equivalents thereof, of the AAV capsid protein. The rAAV can comprise one or more of amino acid substitutions at position N272, S386, and W503. The rAAV can comprise one or more of amino acid substitutions N272A, S386A, W503A, and W503R.

[symbol]

some embodiments, the rAAV vector genome comprises one or more miRNA-122 (miR-122) binding sites. In some embodiments, the one or more miR-122 binding sites are located in the 3′ UTR of the rAAV vector genome.

Disclosed herein include compositions comprising an AAV targeting peptide, an AAV capsid protein, a nucleic acid, an rAAV described herein, or a combination thereof. In some embodiments, the composition is a pharmaceutical composition. In some embodiments, the composition comprises one or more pharmaceutical acceptable carriers.

Disclosed herein include compositions for use in the delivery of an agent to a target environment of a subject in need. In some embodiments, the composition for use comprises an AAV comprising: (1) an AAV capsid protein as disclosed herein and (2) an agent to be delivered to the target environment of the subject. In some embodiments, the target environment is the nervous system. In some embodiments, the target environment is the central nervous system (CNS), the peripheral nervous system (PNS), or a combination thereof. In some embodiments, the target environment is brain endothelial cells, neurons, capillaries in the brain, arterioles in the brain, arteries in the brain, or a combination thereof. In some embodiments, the composition is a pharmaceutical composition. In some embodiments, the composition comprises one or more pharmaceutical acceptable carriers. In some embodiments, the agent to be delivered comprises a nucleic acid, a peptide, a small molecule, an aptamer, or a combination thereof.

In some embodiments, the nucleic acid comprises one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

In some embodiments, the subject in need is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. In some embodiments, the lysosomal storage disorder is Krabbe disease, Sandhoff disease, Tay-Sachs, syndrome, Pompe Disease, or Batten disease. In some embodiments, the subject in need is a subject suffering from, at risk to develop, or has suffered from a stroke, traumatic brain injury, epilepsy, or spinal cord injury.

In some embodiments, the composition is for intravenous administration. In some embodiments, the composition is for systemic administration. In some embodiments, the agent is delivered to endothelial lining of the ventricles in the brain, central canal of the spinal cord, capillaries in the brain, arterioles in the brain, arteries in the brain, or a combination thereof of the subject. In some embodiments, the subject is an adult animal.

Disclosed herein include methods of delivering an agent to a nervous system of a subject. In some embodiments, the method comprises: providing an AAV vector comprising an AAV capsid protein disclosed herein. In some embodiments, the AAV vector further comprises an agent to be delivered to the nervous system. In some embodiments, the method comprises administering the AAV vector to the subject.

In some embodiments, the administration is a systemic administration. In some embodiments, the administration is an intravenous administration. In some embodiments, the subject is a primate and the agent is delivered to the endothelial cells and neurons of the nervous system. In some embodiments, the agent is delivered to the endothelial cells of the nervous system of the subject at least 1.5-fold, 2-fold, or 3-fold more efficiently than the delivery of the agent to the neurons of the nervous system. In some embodiments, the nervous system is the central nervous system (CNS).

Disclosed herein include methods of delivering an agent to a cell. In some embodiments, the method comprises: contacting an AAV vector comprising an AAV capsid protein disclosed herein with the cell. In some embodiments, the AAV vector further comprises an agent to be delivered to the nervous system. In some embodiments, the cell is an endothelial cell or a neuron.

In some embodiments, contacting the AAV vector with the cell occurs in vitro, in vivo or ex vivo. In some embodiments, the cell is present in a tissue, an organ, or a subject. In some embodiments, the cell is a brain endothelial cell, a neuron, a cell in the capillaries in the brain, a cell in the arterioles of the brain, a cell in the arteries in the brain, a cell in the brain vasculature, or a combination thereof. In some embodiments, the agent to be delivered comprises a nucleic acid, a peptide, a small molecule, an aptamer or a combination thereof. In some embodiments, the nucleic acid encodes a therapeutic protein.

In some embodiments, the nucleic acid comprises one or more of: a) a DNA sequence that encodes a trophic factor, a growth factor, or other soluble factors capable of being surrounding cells; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

In some embodiments, the AAV vector is an AAV9 vector, or a variant thereof. In some embodiments, the AAV vector is a vector selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

In some embodiments, the nucleic acid comprises one or more miRNA-122 (miR-122) binding sites. In some embodiments, at least one of the one or more miR-122 binding sites is located in the 3′ UTR of the nucleic acid.

### BRIEF DESCRIPTION OF THE DRAWINGS

FIG. 1A-FIG. 1E depict non-limiting exemplary embodiments of engineered AAVs that can specifically target brain endothelial cells in mice following systemic delivery. FIG. 1A shows a schematic overview of some embodiments of the engineering and characterization of the capsids disclosed herein: (1) and (2) show evolution of AAV9 using the Multiplexed-CREATE method and identification of a novel vector, AAV-X1, that transduces brain endothelial cells specifically and efficiently following systemic administration in mice, (3) depicts combinatorial peptide substitution and point mutation to further refine the novel vector's tropism, yielding improved vectors, (4) shows transfer of the X1 peptide to the AAV1 backbone to enable serotype switching for sequential AAV administration, (5) shows utilization of AAV-X1 to transform the brain endothelial cells into a biofactory, producing Hevin for the CNS, (6) depicts validation of the novel AAVs across rodent models (genetically diverse mice strains and rats), non-human primates (NHPs, marmosets and macaques), and ex vivo human brain slices. FIG. 1B shows representative images of AAV (AAV9, AAV-X1) vector-mediated expression of eGFP in the brain (scale bar, 2 mm) (Top) and zoomed-in images of AAV-X1-mediated expression of eGFP across brain regions (Bottom) including the cortex, hippocampus, thalamus, and midbrain (scale bar, 50 μm). (C57BL/6J, n=3 per group, 3E11 vg IV dose per mouse, 3 weeks of expression). FIG. 1C shows representative images of AAV (AAV9, PHP.V1, BR1, (scale bar, 50 μm). Top row shows GFP-only image and bottom displays GFP, Glut1 and DAPI. FIG. 1D shows percentage of AAV-mediated eGFP-expressing cells that overlap with the Glut1+ marker across brain regions, representing the efficiency of the vectors' targeting of Glut1+ cells. A two-way ANOVA and Tukey's multiple comparisons tests with adjusted P values are reported (****P<0.0001 for AAV9 versus X11 in cortex, hippocampus, and thalamus). Each data point shows the mean±s.e.m of 3 slices per mouse. FIG. 1E shows a graph of the percentage of Glut1+ markers in AAV-mediated eGFP-expressing cells across brain regions, representing the specificity of the vectors' targeting of Glut1+ cells. (C57BL/6J, n=3 per group, 3×10¹¹ vg IV dose per mouse, 3 weeks of expression).

FIG. 2A-FIG. 2D show semi-rational refinement of X1's tropism by further cargo and capsid engineering. FIG. 2A depicts an exemplary illustration demonstrating cargo and capsid engineering to refine X1's tropism to increase brain targeting and lower liver targeting. FIG. 2B shows representative images of novel vector-mediated expression of eGFP in hippocampus and liver. Images are matched in fluorescence intensity to the X1:CAG-GFP image. Brain scale bar, 100 μm. Liver scale bar, 2 mm. (n≥4 per group, ˜8 week-old C57BL/6J males, 3×10¹¹ vg IV dose per mouse, 3 weeks of expression). A representative image of X1-mediated expression of CAG-eGFP is shown (labeled X1: CAG-eGFP). Also shown is a representative image of X1-mediated eGFP expression with cargo engineering by incorporating microRNA-122 target sites (miR-122TS) in the CAG-GFP genome (labeled as X1: CAG-eGFP-miR122 TS). Further capsid engineering by substitution at AA 452-458 of AAV-X1 yielded X1.1, X1.2 and X1.3. Representative images of hippocampal sections from animals transduced by these vectors are shown (labeled as X1.1:CAG-eGFP, X1.2:CAG-eGFP, and X1.3:CAG-eGFP). Further capsid engineering on AAV-X1 by mutating AA272/AA386/AA503 to alanine to yielded X1.4, X1.6, and X1.5, respectively. Representative images of vector-mediated expression of eGFP are shown (labeled as X1.4:CAG-eGFP, X1.5:CAG-eGFP, and X1.6:CAG-eGFP). FIG. 2C shows data related to percentage of AAV-mediated eGFP-expressing cells that overlap with Glut1+ markers across brain regions, representing the efficiency of the vectors' targeting of Glut1+ cells (Left). A two-way ANOVA and Tukey's multiple comparisons tests with adjusted P values are reported (P=0.0002 for X1 versus X1.1 in the cortex, P=0.0022 for X1 versus X1.1 in the hippocampus, P=0.0049 for X1 versus X1.1 in the thalamus; *P≤0.05, **P≤0.01 and ***P≤0.001 are shown, P>0.05 is not shown). Each data point shows the mean±s.e.m of 3 slices per mouse. Also shown is the percentage of Glut1+ markers in AAV-mediated eGFP-expressing cells across brain regions, representing the specificity of the vectors' targeting of Glut1+ cells (Right). A two-way ANOVA and Tukey's multiple comparisons tests X1 versus X1.1 in the hippocampus, P=0.0413 for X1 versus X1.1 in the thalamus; *P≤0.05, **P≤0.01, ***P≤0.001, n.s. P>0.05). Each data point shows the mean±s.e.m of 3 slices per mouse. FIG. 2D shows data related to AAV vector yields from an established laboratory protocol (See, Example 1 below). One-way analysis of variance (ANOVA) non-parametric Kruskal-Wallis test (approximate P=0.0014), and follow-up multiple comparisons with uncorrected Dunn's test are reported (P=0.0099 for AAV9 versus AAV-X1, P>0.9999 for AAV9 versus AAV-X1.1; n≥4 per group, each data point is the mean of 3 technical replicates, mean±s.e.m is plotted). **P≤0.01, n.s. P>0.05.

FIG. 3A-FIG. 3E show that AAV-X1 and AAV-X1.1 efficiently transduce brain endothelial cells across diverse mice strains and rats. FIG. 3A displays surface plasmon resonance (SPR) plots of PHP.eB, PHP.V1, AAV-X1, and AAV-X1.1 binding to surface-immobilized Ly6a-Fc protein captured on a protein A chip. Shown are binding responses for each vector across a range of vector concentrations as labeled. FIG. 3B depicts a non-limiting exemplary illustration demonstrating the IV administration of AAV-X1 capsid packaged with ssAAV:CAG-GFP genome in genetically diverse mice strains (˜8 week-old young C57BL/6J, BALB/cJ, FVB/NJ and CBA/J, 3×10¹¹ vg per mouse) and IV administration of AAV-X1.1 capsid packaged with ssAAV:CAG-tdTomato genome in Lister Hooded rats (adult, 3×10¹³ vg per rat). FIG. 3C depicts representative brain and liver images of AAV-X1-mediated eGFP expression in C57BL/6J, BALB/cJ, FVB/NJ and CBA/J mice with zoomed-in images of hippocampus and thalamus. Sagittal brain section scale bar, 2 mm. Hippocampus and thalamus scale bar, 100 μm. Liver scale bar, 2 mm. FIG. 3D shows representative images of forebrain and hindbrain of AAV-X1.1-mediated tdTomato expression in Lister Hooded rat. Scale bar, 2 mm, zoom-in image scale bar, 100 μm. FIG. 3E shows representative images of cortex of AAV-X1.1-mediated tdTomato expression in Lister Hooded rat, tissues were co-stained with GLUT1.

FIG. 4A-FIG. 4G show that novel vectors can transform the BBB into a biofactory and, with serotype switching, increase BBB permeability for AAVs in non-permissive strains. FIG. 4A shows a non-limiting illustration of an exemplary embodiment for utilizing serotype switching to increase BBB permeability for AAVs in non-permissive strains. IV administration of AAV1-X1 capsid packaged with ssAAV:CAG-Ly6a or PBS in CBA/J mice (˜8-week-old young adults, 3×10¹¹ vg IV dose/mouse, n=4). After 3 weeks, AAV9-PHP.eB packaged with CAG-eGFP was intravenously administered into CBA/J mice. 3 weeks after the second injection, the brain was collected and imaged. FIG. 4B depicts an illustration of AAV1 monomer structure with the position of a 7-mer insertion disclosed herein at AA 588/589, highlighted (darker portion) (Top). Also shown are sequences of AAV9, AAV1, PHP.B, AAV1-physicochemical properties (Zappo). FIG. 4C shows representative images of AAV1, AAV1-PHP.B and AAV-X1-mediated eGFP expression in the hippocampus (Top) (scale bar, 100 μm). Also shown in the bottom row are zoomed-in images of tissues co-stained with Glut1 and DAPI (nuclei). FIG. 4D depicts a representative image of PHP.eB-mediated eGFP expression in the brain of CBA/J mice (Left) (scale bar, 200 m). Also shown are representative images of the brains of CBA/J mice following sequential administration of either AAV9-X1.1: CAG-Ly6a or AAV1-X1:CAG-Ly6a followed by PHP.eB:CAG-eGFP (Right). FIG. 4E depicts an illustration of an exemplary embodiment of the present disclosure for transforming brain endothelial cells into a biofactory. IV administration of AAV-X1.1 capsid packaged with ssAAV:CAG-Hevin-HA or ssAAV:CAG-GFP genome in Hevin-KO mice (˜4-month-old young adults, 1×10¹² vg IV dose/mouse, n=4). Three weeks post-expression, the mice were anesthetized and perfused and fixation and IHC were performed on the brains. Shown in the bottom right is an illustration of the thalamocortical synapses identified by co-localization of VGLUT2 and PSD95 staining. Thalamocortical synapses are lost in Hevin-KO mice. FIG. 4F depicts representative images of AAV-X1.1 vector-mediated expression of eGFP in the brain (Top). The tissues were co-stained with GLUT1 and DAPI markers (scale bars, 30 μm). Bottom panels show representative images of AAV-X1.1 vector-mediated expression of Hevin in the brain. The tissues were co-stained with HA, GLUT1 and DAPI markers (scale bar, 30 μm). FIG. 4G depicts representative images of a cortical slice stained for PSD95 and VGLUT2 (scale bar, 5 μm). Quantification of colocalized puncta of VGLUT2 and PSD95 in mice administered X1.1:CAG-eGFP and X1.1:CAG-Hevin-HA is shown on the right. One-way analysis of variance (ANOVA) Brown-Forsythe test (****P<0.0001 for WT versus Hevin KO: CAG-eGFP, ***P=0.0006 for Hevin KO: CAG-eGFP versus Hevin KO: CAG-Hevin, n.s. P=0.1156 for WT versus Hevin KO: CAG-Hevin; n=4 per group, each data point is the mean of 15 slices for one animal, mean±s.e.m is plotted).

FIG. 5A-FIG. 5F show that engineered AAVs can efficiently transduce cultured Human Brain Microvascular Endothelial Cells (HBMECs), ex vivo macaque brain slices, and ex vivo human brain slices. FIG. 5A shows representative images of AAV (AAV2, AAV9, AAV-DJ, PHP.eB, PHP.V1, BR1, X1, X1.1, X1.2, X1.3, X1.4, X1.5)-mediated eGFP expression in HBMECs. (AAVs packaged with ssAAV:CAG-eGFP, n=6 per condition, 1 day expression). Also displayed in FIG. 5A (continued) are data related to the percentage of cells transduced by the AAVs. In the condition of MOI:3E4, one-way analysis of variance (ANOVA) non-parametric Kruskal-Wallis test (approximate P<0.0001), and follow-up multiple comparisons with uncorrected Dunn's test are reported (P=0.0004 for AAV9 versus AAV-X1, AAV9 versus AAV-X1.3; n=6 per group, each data point is the mean of 3 technical replicates, mean±s.e.m is plotted). In the condition of MOI:3E3, one-way analysis of variance (ANOVA) non-parametric Kruskal-Wallis test (approximate P<0.0001), and follow-up multiple comparisons with uncorrected Dunn's test are reported (P=0.0082 for AAV9 versus AAV-X1, P=0.0004 for AAV9 versus AAV-X1.1, P=0.0001 for AAV9 versus AAV-X1.2; n=6 per group, each data point is the mean of 3 technical replicates, mean±s.e.m is plotted).**P≤0.01, ***P≤0.001, ****P≤0.0001 are shown, P>0.05 is not shown. FIG. 5B depicts a non-limiting exemplary illustration of AAV testing in ex vivo macaque and human brain slices. The brain slices were freshly extracted from southern pig-tailed macaque brain, rhesus macaque brain, and human brain. The slices were cultured at physiological conditions ex vivo. In the pool testing pipeline shown in the top flow diagram, a pool of AAVs packaged with CAG-FXN-HA genome containing a unique barcode was applied to the slice, and DNA extraction and RNA extraction were performed after 7 days. Next-generation sequencing (NGS) was performed to determine the proportion of each barcode (AAV) in DNA and RNA. In the individual testing pipeline shown in the bottom flow diagram, AAVs packaged with CAG-FXN-HA were individually applied to the slices. Fixation, IHC, and imaging were performed on the slices after 7 days. FIG. 5C shows exemplary data related to DNA and RNA level in southern pig-tailed macaque brain slices for AAVs, with DNA and RNA levels normalized to AAV9. FIG. 5D shows representative images of AAV-mediated CAG-FXN-HA expression in ex vivo southern pig-tailed macaque brain slices. The tissues were co-stained with antibodies against HA and NeuN. FIG. 5E shows data related to DNA and RNA level in human brain slices for AAVs, with DNA and RNA levels normalized to AAV9. FIG. 5F depicts an exemplary heatmap of RNA log enrichment of AAVs across pigtailed macaque, rhesus macaque and human brain slices.

FIG. 6A-FIG. 6F show that engineered AAVs can efficiently transduce the central nervous system in rhesus macaque. FIG. 6A shows an illustration of an exemplary embodiment of AAV vector delivery to rhesus macaque to study transduction across the CNS and PNS after 3 weeks of expression. The capsids (AAV9/X1.1) and their corresponding genomes (ssAAV:CAG-eGFP/tdTomato) are shown on the left. Two AAVs packaged with different fluorescent proteins were mixed and intravenously injected at a dose of 5×10¹³ vg/kg per macaque (Macaca mulatta, injected within 10 days of birth, female, i.e. 2.5×10¹³ vg/kg per AAV). FIG. 6B-FIG. 6C show representative images of macaque coronal sections of forebrain, midbrain, hindbrain and cerebellum (scale bar, 2 mm) (FIG. 6B), and, selected brain areas including cortex, lingual gyrus (LG), hippocampus and cerebellum (scale bar, 200 μm) (FIG. 6C). FIG. 6D shows representative images of brain tissues co-stained with NeuN or S100beta or shown by percentage of Fluorescent+/Marker+. Each data point is a slice. FIG. 6F shows exemplary data related to quantification of the fold change of Fluorescent+/NeuN+ over mean AAV9 in the macaque brain. Each data point is a slice.

FIG. 7A-FIG. 7E show detailed characterization of engineered AAVs in the mouse brain. FIG. 7A shows representative images of AAV-X1 vector-mediated expression of eGFP (top row) in the brain. The tissues were co-stained with CD31 marker (middle row) (scale bar, 50 μm). FIG. 7B shows exemplary data related to the percentage of AAV-mediated eGFP-expressing cells that overlap with CD31+ markers across brain regions, representing the efficiency of the vectors in targeting CD31+ cells (Left). Each data point shows the mean±s.e.m of 3 slices per mouse. Also shown is exemplary data related to percentage of CD31+ markers in AAV-mediated eGFP-expressing cells across brain regions, representing the specificity of the vectors in targeting CD31+ cells (Right). (n≥4 per group, ˜8 weeks old C57BL/6J males, 3×10¹¹ vg IV dose per mouse, 3 weeks of expression). FIG. 7C-FIG. 7E depict non-limiting exemplary data showing EGFP expression is seen only in endothelial cells possessing BBB characteristics, and not in other vascular cells. FIG. 7C displays representative images showing PLVAP-positive endothelial cells in choroid plexus (CP) do not express eGFP. The ventricular border is indicated by the dashed line. FIG. 7D-FIG. 7E show representative images of brain sections co-stained with endothelial cell marker (podocalyxin,) and (with arrowheads) markers for smooth muscle cells (calponin 1), perivascular macrophages (CD206), astrocytes (GFAP), and pericytes (CD13). The dashed lines indicate the cortical surface.

FIG. 8A-FIG. 8D show engineered AAVs' expression in peripheral organs and in different physiological conditions. FIG. 8A shows representative images of AAV9, AAV-X1, and AAV-X1.1 vector-mediated expression of eGFP in the small intestine (scale bar, 500 μm), heart (scale bar, 1000 μm) and lung (scale bar, 1000 μm) (n≥4 per group, ˜8 week-old C57BL/6J males, 3×10¹¹ vg IV dose per mouse, 3 weeks of expression). FIG. 8B shows an illustration of an experiment with dye perfusion to evaluate the intactness of the BBB in AAV-injected mice. On the bottom is shown representative images of dye staining in hippocampus and thalamus (scale bars, 300 m) (n=3 per group, 1×10¹² vg IV dose per mouse, 3 weeks of expression). FIG. 8C-FIG. 8D displays representative images of AAV-X1 vector-mediated expression of eGFP in the brains of both sexes and both young 2.5-month-old and aged 2.5-year-old C57BL/6J mice (scale bars: 2 mm in whole brain and 50 m in cortex/hippocampus) (n=3 per group, 1×10¹² vg IV dose per mouse, 3 weeks of expression).

FIG. 9A-FIG. 9D show that engineered AAVs are independent of Ly6a and show different expression patterns in pericyte-deficient mice. FIG. 9A shows representative plasmids encoding Ly6a. (Packaged with ssAAV:CAG-eGFP, n=3 per condition, 2-day expression, high dose: MOI 25000, low dose: MOI 2500). Scale bar, 200 μm. FIG. 9B displays an exemplary illustration of AAV vector delivery to control mice and pericyte-deficient mice (Pdgfb ret/ret) for studying their transduction profile in BBB in different conditions (3×10¹¹ vg/mouse for X1, 1×10¹² vg/mouse for X1.1, tail vein injection, 3 weeks expression). FIG. 9C depicts representative images of AAV-mediated expression of eGFP in coronal sections of mouse brain (scale bar, 1000 μm), and zoomed-in images of tissue co-stained with collagen IV marker (scale bar, 2 mm). FIG. 9D shows representative images of tissue co-stained with GFAP marker (scale bar, 50 μm). Boxes show further zoomed-in views of astrocytes that have endfeet on the vasculature; white arrows highlight the colocalization of GFP expression and GFAP marker in Pdgfb ret/ret mice.

FIG. 10A-FIG. 10E show that engineered AAVs efficiently transduce human cell lines and ex vivo macaque slices. FIG. 10A shows representative images of AAV (AAV9, X1, X1.1, X1.2, X1.3, X1.4, X1.5, X1.6)-mediated eGFP expression in HeLa cells, U87 cells, and IMR32 cells (AAVs packaged with ssAAV:CAG-eGFP, n=3 per condition, 1 day expression, MOI: 50000). FIG. 10B shows exemplary data related to DNA and RNA level in southern pig-tailed macaque brain slices for AAVs, with DNA and RNA levels normalized to AAV9. FIG. 10C shows representative images of AAV (CAP-B1, CAP-B2, CAP-B8, CAP-B18)-mediated CAG-FXN-HA expression in ex vivo southern pig-tailed macaque brain slices. The tissues were co-stained with antibodies against HA and NeuN. FIG. 10D-FIG. 10E show representative images of AAV9 and X1.1-mediated CAG-FXN-HA expression in ex vivo southern pig-tailed macaque brain slices. The tissues were co-stained with antibodies against HA and Satb2, Glut1, S100beta or Olig2.

FIG. 11A-FIG. 11C show that engineered AAV transduces the central nervous system in marmoset similarly to AAV9. FIG. 11A depicts an exemplary illustration of AAV vector delivery to adult marmoset to study transduction across the CNS and PNS after 3 weeks of expression. The capsids (AAV9/X1.1) and their corresponding genomes (ssAAV:CAG-eGFP/tdTomato) are shown on the left. Two AAV vectors packaged with different colored fluorescent reporters were mixed and intravenously delivered at a total dose of 7×10¹³ vg/kg per adult marmoset (16 month-old Callithrix jacchus, 3.5×10¹³ vg/kg per AAV). Representative images of coronal brain sections of the midbrain (scale bar, 2 mm) are shown in FIG. 11B showing AAV9 vector-mediated expression of eGFP (top left) or tdTomato (top right), X1.1-mediated expression of eGFP (bottom left) and X1.1-mediated expression of tdTomato (bottom right). FIG. 11C depicts representative images of select brain areas (cortex and row) or tdTomato (last row), X1.1-mediated expression of eGFP (first row) and X1.1-mediated expression of tdTomato (third row).

FIG. 12 shows representative images of AAV (AAV9, AAV-X1, AAV-X2, and AAV-X3) vector-mediated expression of eGFP in the brain (scale bar, 2 mm) (Left) and zoomed-in images of eGFP across brain regions are shown to the right including the cortex, hippocampus, thalamus, and midbrain (scale bar, 50 μm). (C57BL/6J, n=3 per group, 3×10¹¹ vg IV dose per mouse, 3 weeks of expression).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include AAVs exhibiting tropism for the nervous system (e.g., the central nervous system). In some embodiments, the AAVs comprise one or more targeting peptides of the disclosure. In some embodiments, the targeting peptide comprises an amino acid sequence that comprises at least 4 contiguous amino acids from a sequence selected from the group consisting of GNNTRSV (SEQ ID NO: 13), GNNTRDT (SEQ ID NO: 14) and TNSTRPV (SEQ ID NO: 15).

Provided herein include AAV capsid proteins. In some embodiments, the AAV capsid proteins comprise an AAV targeting peptide disclosed herein. There are also provided nucleic acids comprising a sequence encoding any of the AAV targeting peptides and/or AAV capsid proteins disclosed herein.

Disclosed herein include recombinant adeno-associated viruses (rAAV). In some embodiments, the rAAV comprises one or more of the AAV targeting peptides disclosed herein and/or one or more of the AAV capsid proteins described herein.

[symbol]

an AAV targeting peptide or AAV capsid protein described herein. In some embodiments, the rAAV comprises an AAV capsid protein. In some embodiments, the AAV capsid protein comprises an AAV targeting peptide described herein. In some embodiments, the amino acid sequence is inserted between two adjacent amino acids in AA586-592, or functional equivalents thereof, of the AAV capsid protein.

There are provided compositions comprising an AAV targeting peptide, an AAV capsid protein, a nucleic acid, an rAAV, as described herein, or a combination thereof.

Disclosed herein include compositions for use in the delivery of an agent to a target environment of a subject in need. In some embodiments, the composition comprises an AAV comprising (1) an AAV capsid protein disclosed herein and (2) an agent to be delivered to the target environment of the subject. In some embodiments, the target environment is the nervous system. The agent can be, for example, a nucleic acid, polypeptide, small molecule, or a combination thereof.

Disclosed herein include methods of delivering an agent to a nervous system of a subject. In some embodiments, the method comprises: providing an AAV vector comprising an AAV capsid protein disclosed herein. In some embodiments, the AAV vector comprises an agent to be delivered to the nervous system. In some embodiments, the method comprises administering the AAV vector to the subject. Disclosed herein include methods of delivering an agent to a cell. In some embodiments, the method comprises: contacting an AAV vector comprising an AAV capsid protein disclosed herein with the cell. In some embodiments, the AAV vector comprises an agent to be delivered to the nervous system. In some embodiments, the cell is an endothelial cell or a neuron.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “variant” can refer to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene, for example, XL.D1c-AAV9 and XL.N1-AAV9. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wild type, or naturally occurring. In some instances the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be a recombinant AAV genome generated using recombinatorial genetics methods, and which can include a heterologous nucleic acid (e.g., transgene) that comprises and/or is flanked by the ITR sequences.

[symbol]

recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV inverted terminal repeat (ITR) sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” or “wild type” can be used interchangeably, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The neurovascular unit (NVU) is a vital yet understudied component of the nervous system. Malfunction of non-neuronal cell types within the NVU, including endothelial cells, can facilitate the progression of neurological disorders, but limited options for cell-type specific transgene delivery hamper its study. AAV vectors have emerged as a promising choice for gene delivery, however, AAV vectors for gene delivery to the brain are commonly administered via intra-cranial injections, resulting in tissue damage and limited, uneven spatial coverage. Systemic AAV delivery provides a non-invasive, brain-wide alternative for genetic access. Vectors have been engineered that efficiently cross the blood-brain-barrier (BBB) with broad tropism in rodents (e.g., AAV-PHP.eB). AAV-PHP.V1 is a capsid variant biased towards transducing brain vascular cell following intravenous (I.V.) delivery. Various engineered AAV vectors that can cross BBB have been described in detail in WO2015/038958, WO2017/100671, and WO2020/210655, which are hereby incorporated by reference in their entirety.

[symbol]

under the control of the ubiquitous CAG promoter transduces ˜60% of GLUT1+ cortical brain vasculature, compared with no transduction with AAV9. However, for applications that require endothelial-cell-restricted transduction, PHP.V1 needs to be paired with endothelial specific promoter, and the entrance of the capsid to other cell types may still induce capsid-mediated immune response creating confounding effects. The enhanced central nervous system (CNS) tropism of AAV-PHP.V1 as compared to AAV9 is also absent in a subset of mouse strains including Balb/c. Furthermore, AAV-PHP.V1 is reliant on Ly6a membrane protein for transduction, which may hamper its potential adaptation in cross-species application. More specific AAV vectors which can overcome cross-species barriers would be critical for both research and clinical aspects of studying brain vasculature. Disclosed herein are cell-type-specific vectors that can access vasculature without targeting other components of the NVU.

As described herein, using M-CREATE directed evolution, a family of endothelial-enriched AAV capsid variants, including one named AAV-CAP.X1 (also referred to herein as AAV-X1 or X1) have been identified and are disclosed herein. Following I.V. injection, AAV-CAP.X1 targets vasculature with high cell-type specificity and efficiency throughout the body, including the brain. After injecting 3×10¹¹ viral genome (vg) total of AAV-CAP.X1 packaging CAG-GFP into adult C57BL/6J mice, 97% (+/−0.8%) of the GFP+ area in the hippocampus are CD31+(demonstrating specificity), and 73% (+/−9.1%) of the CD31+ area in the hippocampus is GFP+(proving efficiency; note that an increased dosage of 1E12 vg per mouse resulted in even greater CD31+ labeling without losing specificity).

In some embodiments, for efficient brain-specific endothelial transduction, point mutations on the AAV-CAP.X1 capsid were introduced yielding a series of new capsid variants (e.g., AAV-X1.4, AAV-X1.5, AAV-X1.6) successfully de-targeting the capsid from the liver. The incorporation of one or more miR-122 target sites into the AAV genome was also included, in some embodiments, to reduce liver expression and maintain efficient brain endothelial transduction. To further determine its tropism landscape with additional engineering in other active site of the capsid surface, additional peptides were substituted at the 452-458 position of AAV-CAP.X1 creating another set of new capsids (AAV-1.1, AAV-1.2, AAV-1.3) with higher efficiency and stability.

The engineered capsids of the present disclosure (e.g., AAV-X1) can be used across multiple genetically diverse mouse strains, with efficient labeling of both capillaries and arteries in the brains of C57BL/6J, FVB/NJ, CBA/J, and BALB/cJ mice following I.V. administration. AAV-CAP.X1 also results in a ˜3-fold increase in DNA in the brain compared with AAV9 when I.V. injected together into marmoset. A significant increase in transduction observed. In its brain-targeted form, the AAV capsids of the present disclosure can be paired with pre-clinical therapeutic cargo both to probe vascular contributions to neurological disease and to inform intervention strategies. More broadly, gene delivery via endothelial-tropic AAV capsids can be applied to study diverse pathologies that may benefit from vascular remodeling. For example, the presently disclosed AAV vectors can be advantageously used to investigate the vascular pathology in COVID-19 that could underlie generalized organ dysfunction.

Through a combination of directed evolution and semi-rational engineering, a family of novel vectors, including AAV-X1 and AAV-X1.1 were identified, which target brain endothelial cells specifically and efficiently following systemic delivery in mice with a ubiquitous promoter. Unlike, e.g., PHP.eB, the enhanced CNS targeting of these novel vectors was independent of lymphocyte activation protein-6A (Ly6a). The AAVs disclosed herein were characterized across rodent models (genetically diverse mouse strains and rats), non-human primates (marmosets and rhesus macaques), and ex vivo human brain slices, demonstrating superior transduction of the CNS across species. To illustrate the utility of AAV-X1 for CNS delivery of neuroactive proteins, mouse brain endothelial cells were transformed into a biofactory for producing the synaptogenic protein Sparc-like protein 1 (Sparcl1)/Hevin. Hevin is an astrocyte-secreted protein that controls formation of vesicular glutamate transporter 2 (VGluT2)-containing synapses such as thalamocortical synapses. AAV-X1-mediated ectopic expression of Hevin in brain endothelial cells was sufficient to rescue the thalamocortical synaptic loss phenotype of Hevin knockout mice. The transferability of AAV-X1's properties from the AAV9 serotype to AAV1 was also demonstrated, enabling repeated AAV administration to increase CNS transduction.

Disclosed herein include variant AAV capsid proteins. In some embodiments, the variant capsid comprises an insertion of amino acid sequence comprising the sequence of GNNTRSV (SEQ ID NO: 13) insertion at amino acid position 588/589 of AAV9 VP1 (SEQ ID NO: 1). In some embodiments, the variant capsid protein can comprise an insertion of an amino acid sequence comprising GNNTRSV (SEQ ID NO: 13) at amino acid position 588/589 of AAV9 VP1 and one or more amino acid transitions comprising N272A, S386A, W503A, or W503R mutation in AAV9 VP1, or any combination thereof. In some embodiments, the variant capsid comprises an insertion of an amino acid sequence comprising GNNTRSV (SEQ ID NO: 13) at amino acid position 588/589 of AAV9 VP1 and a substitution at amino acid positions 452-458 of AAV9 VP1 comprising a sequence of LQTSSPG, DGAATKN, or DGQSSKS. In some embodiments, the AAV comprises a viral genome comprising one or more miR-122 target sites.

[symbol]

herein for delivery of nucleic acid molecules, therapeutic proteins, small therapeutic molecules, or other agents in vitro and in vivo.

### Targeting Peptides and Capsid Proteins

Disclosed herein includes AAV vectors comprising an AAV targeting peptide that comprises an amino acid sequence comprising at least 4 contiguous amino acids from the sequence GNNTRSV (SEQ ID NO: 13). In some embodiments, the targeting peptide comprises at least 4 contiguous amino acids from the sequence GNNTRSV (SEQ ID NO: 13). For example, the targeting peptide can comprise the sequence of GNNT (SEQ ID NO: 29), NNTR (SEQ ID NO: 30), NTRS (SEQ ID NO: 31), or TRSV (SEQ ID NO: 32). The targeting peptide can comprise at least 5 contiguous amino acids from the sequence of GNNTRSV (SEQ ID NO: 13). For example, the targeting peptide can comprise the sequence of GNNTR (SEQ ID NO: 33), NNTRS (SEQ ID NO: 34), or NTRSV (SEQ ID NO: 35). The targeting peptide can comprise at least 6 contiguous amino acids from the sequence of GNNTRSV (SEQ ID NO: 13). For example, the targeting peptide can comprise the sequence of GNNTRS (SEQ ID NO: 36), or NNTRSV (SEQ ID NO: 37). In some embodiments, the targeting peptide comprises GNNTRSV (SEQ ID NO: 13).

Disclosed herein is an AAV vector comprising an amino acid sequence that comprises at least 4 contiguous amino acids from the sequence GNNTRDT (SEQ ID NO: 14). Disclosed herein include AAV targeting peptides. In some embodiments, the targeting peptide comprises an amino acid sequence that comprises at least 4 contiguous amino acids from the sequence GNNTRDT (SEQ ID NO: 14). For example, the targeting peptide can comprise the sequence of GNNT (SEQ ID NO: 29), NNTR (SEQ ID NO: 30), NTRD (SEQ ID NO: 38) or TRDT (SEQ ID NO: 39). The targeting peptide can comprise at least 5 contiguous amino acids from the sequence of GNNTRDT (SEQ ID NO: 14). For example, the targeting peptide can comprise the sequence of GNNTR (SEQ ID NO: 33), NNTRD (SEQ ID NO: 40), or NTRDT (SEQ ID NO: 41). The targeting peptide can comprise at least 6 contiguous amino acids from the sequence of GNNTRDT (SEQ ID NO: 14). For example, the targeting peptide can comprise the sequence of GNNTRD (SEQ ID NO: 42) or NNTRDT (SEQ ID NO: 43). In some embodiments, the targeting peptide comprises GNNTRDT (SEQ ID NO: 14).

Disclosed herein is an AAV vector comprising an amino acid sequence that comprises at least 4 contiguous amino acids from the sequence TNSTRPV (SEQ ID NO: 15). Disclosed herein include AAV targeting peptides. In some embodiments, the targeting peptide comprises an amino acid sequence that comprises at least 4 contiguous amino acids from the sequence TNSTRPV (SEQ ID NO: 15). For example, the targeting peptide can comprise the TRPV (SEQ ID NO: 47). The targeting peptide can comprise at least 5 contiguous amino acids from the sequence of TNSTRPV (SEQ ID NO: 15). For example, the targeting peptide can comprise the sequence of TNSTR (SEQ ID NO: 48), NSTRP (SEQ ID NO: 49), or STRPV (SEQ ID NO: 50). The targeting peptide can comprise at least 6 contiguous amino acids from the sequence of TNSTRPV (SEQ ID NO: 15). For example, the targeting peptide can comprise the sequence of TNSTRP (SEQ ID NO: 51) or NSTRPV (SEQ ID NO: 52). In some embodiments, the targeting peptide comprises TNSTRPV (SEQ ID NO: 15).

The targeting AAV peptide can be part of an AAV, for example part of a capsid protein of the AAV. In some embodiments, the capsid protein is the VP1 capsid protein. The targeting peptide can be conjugated to a nanoparticle, a second molecule, a viral capsid protein, or a combination thereof. The targeting peptide can be a central nervous system (CNS) targeting peptide.

Disclosed herein include AAV capsid proteins. In some embodiments, the AAV capsid protein comprises an AAV targeting peptide disclosed herein.

The AAV capsid protein can comprise a substitution, for example an substitution of 7, 6, 5, 4, 3, or 2 contiguous amino acids. The AAV capsid protein can comprise at least 4 contiguous amino acids from a second amino acid sequence selected from DGQSSKS (SEQ ID NO: 17), DGAATKN (SEQ ID NO: 16), and LQTSSPG (SEQ ID NO: 18). For example, the second amino acid sequence can comprise the sequence of DGQS (SEQ ID NO: 62), GQSS (SEQ ID NO: 63), QSSK (SEQ ID NO: 64), SSKS (SEQ ID NO: 65), DGAA (SEQ ID NO: 53), GAAT (SEQ ID NO: 54), AATK (SEQ ID NO: 55), ATKN (SEQ ID NO: 56), LQTS (SEQ ID NO: 71), QTSS (SEQ ID NO: 72), TSSP (SEQ ID NO: 73), or SSPG (SEQ ID NO: 74).

The AAV capsid protein can comprise at least 5 contiguous amino acids from a second amino acid sequence selected from DGQSSKS (SEQ ID NO: 17), DGAATKN (SEQ ID NO: 16), and LQTSSPG (SEQ ID NO: 18). For example, the second amino acid sequence can comprise the sequence of DGQSS (SEQ ID NO: 66), GQSSK (SEQ ID NO: 67), QSSKS (SEQ ID NO: 68), DGAAT (SEQ ID NO: 57), GAATK (SEQ ID NO: 58), AATKN (SEQ ID NO: 59), LQTSS (SEQ ID NO: 75), QTSSP (SEQ ID NO: 76), or TSSPG (SEQ ID NO: 77).

The AAV capsid protein can comprise at least 6 contiguous amino acids from a second amino acid sequence selected from DGQSSKS (SEQ ID NO: 17), DGAATKN (SEQ ID NO: 16), and LQTSSPG (SEQ ID NO: 18). For example, the targeting peptide can comprise the sequence of DGQSSK (SEQ ID NO: 69), GQSSKS (SEQ ID NO: 70), DGAATK (SEQ ID 79).

The AAV capsid protein can comprise a second amino acid sequence comprising DGQSSKS (SEQ ID NO: 17), DGAATKN (SEQ ID NO: 16), and LQTSSPG (SEQ ID NO: 18). The location of the at least 4, 5 or 6 contiguous amino acids from the second amino acid sequence, or the second amino acid sequence, in the AAV capsid protein can vary. In some embodiments, the at least 4, 5 or 6 contiguous amino acids from the second amino acid sequence replace at least 4, 5, 6 or 7 amino acids in AA452-458, or functional equivalents thereof, of the AAV capsid protein. In some embodiments, the at least 4, 5 or 6 contiguous amino acids from the second amino acid sequence, or the second amino acid sequence, replace at least 4, 5, 6 or 7 amino acids in the 455 loop, or functional equivalents thereof, of the AAV capsid protein.

In some embodiments, the AAV capsid protein comprises one or more amino acid substitutions. The AAV capsid protein can comprise one or more of amino acid substitutions at position N272, S386, and W503. In some embodiments, the AAV capsid protein comprises one or more of amino acid substitutions N272A, S386A, W503A, and W503R.

The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid protein can be derived from an AAV serotype selected from AAV9, AAV9 K449R (or K449R AAV9), AAV1, AAVrh1O, AAV-DJ, AAV-DJ8, AAV5, AAVPHP.B (PHP.B), AAVPHP.A (PUPA), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 A1 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3. 1/hu.6, AAV3. 1/hu.9, AAV3-9/rh.52, AAV3-1 1/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.1 1, AAV29.3/bb.1, AAV29.5/bb.2, AAV106. 1/hu.37, AAV1 14.3/hu.40, AAV127.2/hu.41, AAV127.5/hu.42, AAV128.3/hu.44, AAV130.4/hu.48, AAV145. 1/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi. 1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu. 1, AAVhu.2, AAVhu.3, AAVhu.4, AAVhu.5, AAVhu.6, AAVhu.7, AAVhu.9, AAVhu.10, AAVhu. 1 1, AAVhu. 13, AAVhu.15, AAVhu.16, AAVhu. 1 7, AAVhu.1 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 1 4/9, AAVhu.t 19, AAVrh.2, AAVrh.2R, AAVrh.8, AAVrh.8R, AAVrh. 1O, AAVrh.12, AAVrh. 13, AAVrh.13R, AAVrh. 14, AAVrh.17, AAVrh. 18, AAVrh.19, AAVrh.20, AAVrh.21, AAVrh.22, AAVrh.23, AAVrh.24, AAVrh.25, AAVrh.3 1, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhEl.1, AAVhEr1.5, AAVhER1. 14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.3 1, AAVhEr2.36, AAVhER1.23, AAVhEr3.1, AAV2.5T, AAV-PAEC, AAV-LKO1, AAV-LK02, AAV-LK03, AAV-LK04, AAV-LK05, AAV-LK06, AAV-LK07, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu. 1 1, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128. 1/hu.43, true type AAV (ttAAV), EGRENN AAV 10, Japanese AAV 10 serotypes, AAV CBr-7.1, AAV CBr-7. 10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6. 1, AAV CHt-6.1O, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLvl-1, AAV Clvl-1O, AAV CLvl-2, AAV CLv-12, AAV CLvl-3, AAV CLv-1 3, AAV CLvl-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-Kl, AAV CLv-K3, AAV CLv-K6, AAV CLv-L4, AAV CLv-L5, AAV CLv-L6, AAV CLv-M1, AAV CLv-M1 1, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-1O, AAV CSp-1 1, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, thereof. The rAAV disclosed herein can have a capsid from a different serotype of AAV than the rAAV genome.

The engineered AAV capsid proteins described herein have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein.

The rAAV can comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV may be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype is pseudotyped, which means that the ITRs from a first AAV serotype (e.g., AAV1) are used in a capsid from a second AAV serotype (e.g., AAV9), wherein the first and second AAV serotypes are not the same. As a non-limiting example, a pseudotyped AAV serotype comprising the AAV1 ITRs and AAV9 capsid protein may be indicated AAV1/9. The rAAV may additionally, or alternatively, comprise a capsid that has been engineered to express an exogenous ligand binding moiety (e.g., receptor), or a native receptor that is modified.

In some embodiments, the rAAV capsid proteins comprises a substitution or insertion of one or more amino acids in an amino acid sequence of an AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from the group consisting of AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

### Adeno-Associated Virus (AAV) Vectors and Recombinant AAVs

AAV is a replication-deficient parvovirus, the single-stranded DNA genome of which is about 4.7 kb in length including 145 nucleotide ITRs. The ITRs play a role in integration of the AAV DNA into the host cell genome. When AAV infects a host cell, the viral genome integrates into the host's chromosome resulting in latent infection of the cell. In a natural system, a helper virus (e.g., adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating. Disclosed herein include recombinant AAV (rAAV). In some embodiments, the rAAV comprises an AAV capsid protein described herein.

In some embodiments, the AAV vector comprises coding regions of one or more proteins of interest. The AAV vector can include a 5′ ITR of AAV, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulator-element downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook el al. (Molecular Cloning: A Laboratory Manual. Cold Spring Harbor Laboratory Press, N.Y. (1989)). The viral vector can incorporated in their native form or can be modified in any way to obtain a desired activity. For example, the sequences can comprise insertions, deletions or substitutions.

The viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

Vectors comprising a nucleic acid sequence encoding the modified AAV capsid proteins of the present disclosure are also provided herein. For example, the vectors of the present disclosure can comprise a nucleic acid sequence encoding the two AAV viral genes, Rep (Replication), and a Cap (Capsid) gene, wherein the Cap gene, encoding viral capsid proteins VP1, VP2, and VP3 is modified to produce the modified AAV capsid proteins of the present disclosure.

Disclosed herein are methods of producing a rAAV. In some embodiments, all elements that are required for AAV production in target cell (e.g., HEK293 cells) are transiently transfected into the target cell using suitable methods known in the art. For example, the rAAV of the present disclosure can be produced by co-transfecting three plasmid vectors, a first vector with ITR-containing plasmid carrying the transgene (e.g., a DNA sequence that encodes a trophic factor, a growth factor, or other soluble factors), a second vector that carries the AAV Rep and Cap genes (e.g., one or more variant capsid proteins provided herein); and (3), a third vector that provides the helper genes isolated from adenovirus. In some cases, rAAVs of the present disclosure are generated using the methods described in Challis et al. Systemic AAV vectors for widespread and targeted gene delivery in rodents. Nat. Protoc. 14, 379 (2019), which is hereby incorporated by reference in its entirety. Briefly, triple transfection of HEK293T cells using polyethylenimine (PEI) is performed, viruses are collected after 120 hours from both cell lysates and media and purified over iodixanol.

Disclosed herein, are methods of manufacturing comprising: (a) introducing into a cell a nucleic acid comprising: (i) a first nucleic acid sequence (heterologous nucleic acid) encoding, e.g., a protein, enclosed by a 5′ and a 3′ inverted terminal repeat (ITR) sequence; (ii) a second nucleic acid sequence encoding a viral genome comprising a 5′ ITR sequence, a Replication (Rep) gene, one or more (Cap) genes, and a 3′ ITR, wherein the one or more Cap sequence encoding a first helper virus protein selected from the group consisting of E4orf6, E2a, and VA RNA, and optionally, a second helper virus protein comprising Ela or Elb55k; (b) expressing in the cell the AAV capsid protein described herein; (c) assembling an AAV particle comprising the AAV capsid proteins disclosed herein; and (d) packaging the first nucleic acid sequence in the AAV particle. In some instances, the methods further comprise packing the first nucleic acid sequence encoding the therapeutic gene expression product such that it becomes encapsidated by the rAAV capsid protein. In some embodiments, the rAAV particles are isolated, concentrated, and purified using suitable viral purification methods, such as those described herein.

In some embodiments, the rAAVs are generated by triple transfection of precursor cells (e.g., HEK293T) cells using a standard transfection protocol (e.g., with PEI). Viral particles are harvested from the media after a period of time (e.g., 72 h post transfection) and from the cells and media at a later point in time (e.g., 120 h post transfection). Virus present in the media is concentrated by precipitation with 8% poly(ethylene glycol) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The Rep protein can be selected from Rep78, Rep68, Rep52, and Rep40. The genome of the AAV helper virus comprises an AAV helper gene selected from E2, E4, and VA. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The cell can be a cell from a human, a primate, a murine, a feline, a canine, a porcine, an ovine, a bovine, an equine, a caprine and a lupine host cell. The cell can be a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some instances, the embryonic stem cell is a human embryonic stem cell. In some instances, the human embryonic stem cell is a human embryonic kidney 293 (HEK-293) cell. In some instances, the cell is a differentiated cell. Base on the disclosure provided, it is expected that this system can be used in conjunction with develop AAV capsids that more efficiently transduce that target cell population.

There are provided nucleic acids comprising a sequence encoding any of the AAV capsid proteins of the disclosure (e.g., comprising a targeting peptide). For example, there are provided herein plasmid vectors encoding the variant capsid proteins of the present disclosure (e.g., comprising targeting peptides). Also disclosed are nucleic acids encoding the rAAV capsids comprising variant AAV capsid proteins (e.g., comprising targeting peptides) of the present disclosure. Heterologous nucleic acids and transgenes of the present embodiment may also include plasmid vectors. Plasmid vectors are useful for the generation of the rAAV particles described herein. An AAV vector can comprise a genome of a helper virus. Helper virus proteins are required for the assembly of a recombinant rAAV, and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination thereof. In some instances, the plasmid vector is bacterial. In some instances, the plasmid vector is derived from Escherichia coli. In some instances, the nucleic acid sequence comprises, in a 5′ to 3′ direction: (1) a 5′ ITR sequence, (2) a Replication (Rep) gene, (3) a Capsid (Cap) gene, and (4) a 3′ ITR, wherein the Cap gene encodes the variant AAV capsid protein described herein. In some instances, the plasmid vector encodes a pseudotyped AAV capsid protein.

Disclosed herein are modified viral genomes comprising genetic information (e.g., heterologous nucleic acid) that are assembled into a rAAV via viral packaging. In some instances, the viral genome is from an AAV serotype selected from AAV1, AAV2, AAV3, AAV4, AAV5, AAV6, AAV7, AAV8, AAV9, AAV10, AAV 11, and AAV12.

A viral genome, such as those described herein, can comprise a transgene, which in some cases encodes a heterologous gene expression product (e.g., therapeutic gene expression product, recombinant capsid protein, and the like). The transgene is in cis with two ITRs flanking the transgene. The transgene may comprise a therapeutic nucleic acid encoding a therapeutic gene expression product

The viral genome, in some cases, is a single stranded viral DNA comprising the transgene. The AAV vector can be episomal. In some instances, the viral genome is a concatemer. An episomal viral genome can develop chromatin-like organization in the target cell that does not integrate into the genome of the target cell. When infected into non-dividing cells, the stability of the episomal viral genome in the target cell enable the long-term transgene cell predominantly at a specific site (e.g., AAVS 1 on human chromosome 19).

the rAAV genome can, for example, comprise at least one inverted terminal repeat configured to allow packaging into a vector and a cap gene. In some embodiments, it can further include a sequence within a rep gene required for expression and splicing of the cap gene. In some embodiments, the genome can further include a sequence capable of expressing a capsid protein provided herein.

The rAAV capsid proteins can be isolated and purified. The AAV can be isolated and purified by methods standard in the art such as by column chromatography or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

The rAAV capsid and/or rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein. In some instances, the second molecule is a detectable moiety. For example, the modified AAV capsid and/or rAAV capsid protein conjugated to a detectable moiety may be used for in vitro, ex vivo, or in vivo biomedical research applications, the detectable moiety used to visualize the modified capsid protein. The modified AAV capsid and/or rAAV capsid protein conjugated to a detectable moiety may also be used for diagnostic purposes.

One or more insertions, substitutions, or point mutations can be employed in a single system (e.g., in a single AAV vector, a single AAV capsid protein, or a single rAAV). For example one can employ one or more targeting sequences and also modify other sites to reduce the recognition of the AAVs by the pre-existing antibodies present in a subject, such as a human. The AAV vector can include a capsid, which influences the tropism/targeting, speed of expression and possible immune response. The vector can also include the rAAV, which genome carries the transgene/therapeutic aspects (e.g., sequences) along with regulatory sequences. The vector can include the targeting sequence within/on a substrate that is or transports the desired molecule (e.g., therapeutic molecule, diagnostic molecule).

In some embodiments, the rAAV comprises an AAV capsid protein comprising any of the AAV targeting peptide described herein. In some embodiments, the rAAVs exhibit tropism for the central nervous system.

[symbol]

amino acids from the sequence GNNTRSV (SEQ ID NO: 13). For example, the targeting peptide can comprise the sequence of GNNT (SEQ ID NO: 29), NNTR (SEQ ID NO: 30), NTRS (SEQ ID NO: 31), or TRSV (SEQ ID NO: 32). The targeting peptide can comprise at least 5 contiguous amino acids from the sequence of GNNTRSV (SEQ ID NO: 13). For example, the targeting peptide can comprise the sequence of GNNTR (SEQ ID NO: 33), NNTRS (SEQ ID NO: 34), or NTRSV (SEQ ID NO: 35). The targeting peptide can comprise at least 6 contiguous amino acids from the sequence of GNNTRSV (SEQ ID NO: 13). For example, the targeting peptide can comprise the sequence of GNNTRS (SEQ ID NO: 36), or NNTRSV (SEQ ID NO: 37). In some embodiments, the targeting peptide comprises GNNTRSV (SEQ ID NO: 13).

In some embodiments, the targeting peptide comprises an amino acid sequence that comprises at least 4 contiguous amino acids from the sequence GNNTRDT (SEQ ID NO: 14). For example, the targeting peptide can comprise the sequence of GNNT (SEQ ID NO: 29), NNTR (SEQ ID NO: 30), NTRD (SEQ ID NO: 38) or TRDT (SEQ ID NO: 39). The targeting peptide can comprise at least 5 contiguous amino acids from the sequence of GNNTRDT (SEQ ID NO: 14). For example, the targeting peptide can comprise the sequence of GNNTR (SEQ ID NO: 33), NNTRD (SEQ ID NO: 40), or NTRDT (SEQ ID NO: 41). The targeting peptide can comprise at least 6 contiguous amino acids from the sequence of GNNTRDT (SEQ ID NO: 14). For example, the targeting peptide can comprise the sequence of GNNTRD (SEQ ID NO: 42) or NNTRDT (SEQ ID NO: 43). In some embodiments, the targeting peptide comprises GNNTRDT (SEQ ID NO: 14).

In some embodiments, the targeting peptide comprises an amino acid sequence that comprises at least 4 contiguous amino acids from the sequence TNSTRPV (SEQ ID NO: 15). For example, the targeting peptide can comprise the sequence of TNST (SEQ ID NO: 44), NSTR (SEQ ID NO: 45), STRP (SEQ ID NO: 46), or TRPV (SEQ ID NO: 47). The targeting peptide can comprise at least 5 contiguous amino acids from the sequence of TNSTRPV (SEQ ID NO: 15). For example, the targeting peptide can comprise the sequence of TNSTR (SEQ ID NO: 48), NSTRP (SEQ ID NO: 49), or STRPV (SEQ ID NO: 50). The targeting peptide can comprise at least 6 contiguous amino acids from the sequence of TNSTRPV (SEQ ID NO: 15). For example, the targeting peptide can comprise the sequence of TNSTRP (SEQ ID NO: 51) or NSTRPV (SEQ ID NO: 52). In some embodiments, the targeting peptide comprises TNSTRPV (SEQ ID NO: 15).

The location of the targeting peptide within the capsid protein can vary. In some embodiments, the amino acid sequence is inserted between two adjacent amino acids in AA589 and AA590, AA590 and AA591, AA591 and AA592) or functional equivalents thereof, of the AAV capsid protein. The two adjacent amino acids can be AA588 and AA589. In some embodiments, the AAV capsid protein comprises, or consists thereof, SEQ ID NO: 1 or 2 (e.g., the VP1 protein of AAV1 or AAV9).

In some embodiments, the rAAV can further comprise a second amino acid sequence. In some embodiments, the second amino acid sequence can enhance the tropism of the rAAV for the CNS and decrease the tropism of the rAAV for non-neuronal tissues, e.g., the liver. The rAAV can comprise at least 4 contiguous amino acids from a second amino acid sequence selected from the group consisting of DGQSSKS (SEQ ID NO: 17), DGAATKN (SEQ ID NO: 16), and LQTSSPG (SEQ ID NO: 18). For example, the second amino acid sequence can comprise the sequence of DGQS (SEQ ID NO: 62), GQSS (SEQ ID NO: 63), QSSK (SEQ ID NO: 64), SSKS (SEQ ID NO: 65), DGAA (SEQ ID NO: 53), GAAT (SEQ ID NO: 54), AATK (SEQ ID NO: 55), ATKN (SEQ ID NO: 56), LQTS (SEQ ID NO: 71), QTSS (SEQ ID NO: 72), TSSP (SEQ ID NO: 73), or SSPG (SEQ ID NO: 74).

The rAAV can comprise at least 5 contiguous amino acids from a second amino acid sequence selected from the group consisting of DGQSSKS (SEQ ID NO: 17), DGAATKN (SEQ ID NO: 16), and LQTSSPG (SEQ ID NO: 18). For example, the second amino acid sequence can comprise the sequence of DGQSS (SEQ ID NO: 66), GQSSK (SEQ ID NO: 67), QSSKS (SEQ ID NO: 68), DGAAT (SEQ ID NO: 57), GAATK (SEQ ID NO: 58), AATKN (SEQ ID NO: 59), LQTSS (SEQ ID NO: 75), QTSSP (SEQ ID NO: 76), or TSSPG (SEQ ID NO: 77).

The rAAV can comprise at least 6 contiguous amino acids from a second amino acid sequence selected from the group consisting of DGQSSKS (SEQ ID NO: 17), DGAATKN (SEQ ID NO: 16), and LQTSSPG (SEQ ID NO: 18). For example, the targeting peptide can comprise the sequence of DGQSSK (SEQ ID NO: 69), GQSSKS (SEQ ID NO: 70), DGAATK (SEQ ID NO: 60), GAATKN (SEQ ID NO: 61), LQTSSP (SEQ ID NO: 78), or QTSSPG (SEQ ID NO: 79).

The rAAV can comprise an amino acid sequence of DGQSSKS (SEQ ID NO: 17), DGAATKN (SEQ ID NO: 16), or LQTSSPG (SEQ ID NO: 18).

The location of the second amino acid sequence within the capsid protein can vary. In some embodiments, the at least 4, 5, or 6 contiguous amino acids from a second amino acid sequence, or the second amino acid sequence, replace at least 4, 5, 6 or 7 amino acids in AA452-458, or functional equivalents thereof, of the AAV capsid protein. In some embodiments, the at least 4, 5, or 6 contiguous amino acids from a second amino acid sequence, functional equivalents thereof, of the AAV capsid protein. The rAAV can comprise one or more of amino acid substitutions at position N272, S386, and W503, for example N272A, S386A, W503A, and W503R.

The rAAV can comprise an rAAV vector genome, for example a rAAV vector genome comprising one or more miRNA-122 (miR-122) binding sites. The one or more miR-122 binding sites can be located in the 3′ UTR of the rAAV vector genome. In some embodiments, the presence of the one or more miR-122 binding sites can reduce the expression levels of genes encoded on the rAAV vector genome in non-neuronal tissues (e.g., the liver).

The AAVs disclosed herein, in some embodiments, can increase transduction efficiency of AAVs to a target environment (e.g., the CNS) in the subject as compared to a non-variant AAV (e.g., AAV9). For example, the inclusion of one or more of the targeting peptides disclosed herein in an rAAV can result in an increase in transduction efficiency by, or by at least, 10%, 20%, 30%, 40%, 50%, 60%, 70%, 80%, 90%, 100%, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 10-fold, 20-fold, 30-fold, 40-fold, 50-fold, 60-fold, 70-fold, 80-fold, 90-fold, 100-fold, or a number or a range between any two of these values, as compared to a non-variant AAV (e.g., AAV9). In some embodiments, the increase is at least 1.5-fold. In some embodiments, the increase is a 40-90 fold increase. In some embodiments, the transduction efficiency is increased for transducing the variant AAV to the CNS. In some embodiments, the transduction efficiency is increased for transducing the variant AAV to the neurovascular unit. In some embodiments, the transduction efficiency is increased for transducing the variant AAV to endothelial cells, smooth muscle cells, neurons, glia, or any combination thereof.

In some embodiments, AAV genomes contain both the full rep and cap sequence that have been modified so as to not prevent the replication of the virus under conditions in which it could normally replicate (co-infection of a mammalian cell along with a helper virus such as adenovirus). A pseudo wild-type (“wt”) genome can be one that has an engineered cap gene within a “wt” AAV genome. In some embodiments, the “pseudo-wild type” AAV genome contains the viral replication gene (rep) and capsid gene (cap) flanked by ITRs. In some embodiments, the rAAV genome contains the cap gene and only those sequences within the rep gene required for the expression and splicing of the cap gene products. In some embodiments, a capsid gene recombinase recognition sequence is provided with inverted terminal repeats flanking these sequences.

Disclosed herein include compositions comprising an AAV targeting peptide, an AAV capsid protein, a nucleic acid, an rAAV, as described herein, or a combination thereof. Disclosed herein include compositions for use in the delivery of an agent to a target environment of a subject in need. In some embodiments, the composition comprises an AAV comprising (1) an AAV capsid protein disclosed herein and (2) an agent to be delivered to the target environment of the subject. In some embodiments, the target environment is the nervous system. The compositions can be pharmaceutical compositions comprising one or more pharmaceutical acceptable carriers.

The target environment can be the central nervous system (CNS), the peripheral nervous system (PNS), or a combination thereof. The target environment can be brain endothelial cells, neurons, capillaries in the brain, arterioles in the brain, arteries in the brain, or a combination thereof.

The agent to be delivered can comprise a nucleic acid, a peptide, a small molecule, an aptamer, or a combination thereof. The AAV vectors disclosed herein can be effectively transduced to a target environment (e.g., the CNS), for example, for delivering nucleic acids. In some embodiments, a method of delivering a nucleic acid sequence to the nervous system is provided. The protein can be part of a capsid of an AAV. The AAV can comprise a nucleic acid sequence to be delivered to a nervous system. One can then administer the AAV to the subject.

In some embodiments, the nucleic acid sequence to be delivered to a target environment (e.g., nervous system) comprises one or more sequences that would be of some use or benefit to the nervous system and/or the local of delivery or surrounding tissue or environment. In some embodiments, it can be a nucleic acid that encodes a protein of interest. The nucleic acid can comprise one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

[symbol]

elements known to one of skill in the art to influence the expression of the RNA and/or protein products encoded by the polynucleotide within desired cells of the subject.

In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The heterologous nucleic acid can comprise a 5′ ITR and a 3′ ITR. The agent can comprise a DNA sequence encoding a protein (e.g., a trophic factor, a growth factor, or a soluble protein). The heterologous nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. In some embodiments, the silencing effector comprises one or more miRNA binding sites (e.g., miR-122 binding sites). miRNA binding sites are operably linked regulatory elements that are typically located in the 3′UTR of the transcribed sequence. Binding of miRNAs to the target transcript (in complex with the RNA-Induced Silencing Complex, RISC) can reduce expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. In some embodiments, the gene of interest can be selected from the group comprising SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. The heterologous nucleic acid further can comprise a polynucleotide encoding one or more secondary proteins, and the heterologous nucleic acid can comprise a single-stranded AAV (ssAAV) vector or a self-complementary AAV (scAAV) vector.

The promoter can comprise a ubiquitous promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, including but not limited to, a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be a muscle-specific promoter. In some embodiments, the muscle-specific promoter can comprise a MCK promoter.

The promoter can comprise an intronic sequence. The promoter can comprise a bidirectional promoter and/or an enhancer. In some embodiments, the enhancer can be a CMV sequence (e.g., a gene), and the promoter can comprise or can be derived from the promoter of the endogenous version. In some embodiments, one or more cells of a subject comprise an endogenous version of the nucleic acid sequence, and the sequence is not truncated relative to the endogenous version.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 1 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

As used herein, a “protein of interest” can be any protein, including naturally-occurring and non-naturally occurring proteins. In some embodiments, a polynucleotide encoding one or more proteins of interest can be present in one of the AAV vectors disclosed herein, wherein the polynucleotide is operably linked with a promoter. In some instances, the promoter can drive the expression of the protein(s) of interest in a host cell (e.g., an endothelial cell). In some embodiments, the protein of interest is an anti-tau antibody, an anti-AB antibody, and/or ApoE isoform.

[symbol]

survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The protein can comprise a disease-associated protein. In some embodiments, the level of expression of the disease-associated protein correlates with the occurrence and/or progression of the disease. The protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

In some embodiments, the nucleic acid can comprise a cDNA that encodes a protein to control or monitor the activity or state of a cell, and/or for assessing the state of a cell. The protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The protein can comprise a nuclear localization signal (NLS) or a nuclear export signal (NES).

The protein can comprise a CRE recombinase, GCaMP, a cell therapy component, a knock-down gene therapy component, a cell-surface exposed epitope, or any combination thereof. The protein can comprise a chimeric antigen receptor. The protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

[symbol]

protein for gene editing, or a guide RNA; or a DNA sequence for genome editing via homologous recombination. The protein can comprise a programmable nuclease. In some embodiments, the programmable nuclease is selected from the group comprising: SpCas9 or a derivative thereof, VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof, dcas9-APOBEC1 fusion, BE3, and dcas9-deaminase fusions; dcas9-Krab, dCas9-VP64, dCas9-Tetl, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The heterologous nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA).

The rAAV disclosed herein can comprise one or more of the heterologous nucleic acids disclosed herein. The heterologous nucleic acid can comprise a polynucleotide encoding a protein. The nucleic acid can be or can encode an RNA agent. The heterologous nucleic acid can comprise a promoter operably linked to the polynucleotide encoding a protein. As disclosed herein, the gene is operatively linked with appropriate regulatory elements in some embodiments. The one or more genes of the heterologous nucleic acid can comprise an siRNA, an shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. The one or more genes of the heterologous nucleic acid can comprise one or more synthetic protein circuit components. The one or more genes of the heterologous nucleic acid can comprise can entire synthetic protein circuit comprising one or acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof, insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof, clotting factors and variants thereof; cystic fibrosis transmembrane conductance regulator (CFTR) and variants thereof, and interferons and variants thereof.

Examples of protein of interest include, but are not limited to, luciferases; fluorescent proteins (e.g., GFP); growth hormones (GHs) and variants thereof, insulin-like growth factors (IGFs) and variants thereof, granulocyte colony-stimulating factors (G-CSFs) and variants thereof, erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof, clotting factors and variants thereof; CFTR and variants thereof, and interferons and variants thereof.

In some embodiments, the protein of interest is a therapeutic protein or variant thereof. Non-limiting examples of therapeutic proteins include blood factors, such as 0-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (GF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and enzyme activators, such as tissue plasminogen activator; chemokines, such as IP-10, monokine induced by interferon-gamma (Mig), Groa/IL-S, RANTES, MlP-1 a, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-rel easing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of nietalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor IX or Factor X; dystrophin or nini-dystrophm; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

The protein of interest can be, for example, an active fragment of a protein, such as any of the aforementioned proteins, a fusion protein comprising some or all of two or more proteins, or a fusion protein comprising all or a portion of any of the aforementioned proteins.

In some embodiments, the viral vector comprises a polynucleotide comprising coding regions for two or more proteins of interest, The two or more proteins of interest can be the same or different from each other. In some embodiments, the two or more same antibody.

The protein of interest can be a multi-subunit protein. For example, the protein of interest can comprise two or more subunits, or two or more independent polypeptide chains. In some embodiments, the protein of interest can be an antibody, including, but are not limited to, antibodies of various isotypes (for example, IgG1, IgG2, IgG3, IgG, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody. In some embodiments, the protein of interest is not an immunoadhesin.

In some embodiments, the resulting targeting molecules can be employed in methods and/or therapies relating to in vivo gene transfer applications to long-lived cell populations. In some embodiments, these can be applied to any rAAV-based gene therapy, including, for example: spinal muscular atrophy (SMA), amyotrophic lateral sclerosis (ALS), Parkinson's disease, Friedreich's ataxia, Pompe disease, Huntington's disease, Alzheimer's disease, Battens disease, lysosomal storage disorders, glioblastoma multiforme, Rett syndrome, Leber's congenital amaurosis, chronic pain, stroke, spinal cord injury, traumatic brain injury and lysosomal storage disorders. In addition, rAAVs can also be employed for in vivo delivery of transgenes for non-therapeutic scientific studies such as optogenetics, gene overexpression, gene knock-down with shRNA or miRNAs, modulation of endogenous miRNAs using miRNA sponges or decoys, recombinase delivery for conditional gene deletion, conditional (recombinase-dependent) expression, or gene editing with CRISPRs, TALENs, and zinc finger nucleases.

In some embodiments, the gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the heterologous nucleic acids provided herein can be employed as vaccines for the prophylaxis or treatment of infectious diseases (e.g., as vaccines).

As described herein, the nucleotide sequence encoding the protein can be modified to improve expression efficiency of the protein. The methods that can be used to improve the transcription and/or translation of a gene herein are not particularly limited. For gene expression (e.g., protein production) in the host (e.g., a mammal).

The degree of gene expression in the target cell can vary. The amount of the protein expressed in the subject (e.g., the CNS of the subject) can vary. For example, in some embodiments the protein can be expressed in the subject in the amount of at least about 9 μg/ml, at least about 10 μg/ml, at least about 50 μg/ml, at least about 100 μg/ml, at least about 200 g/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the protein is expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein is needed for the method to be effective can vary depending on non-limiting factors such as the particular protein and the subject receiving the treatment, and an effective amount of the protein can be readily determined by a skilled artisan using conventional methods known in the art without undue experimentation.

The agent can be an inducer of cell death. The agent can induce cell death by a non-endogenous cell death pathway (e.g., a bacterial pore-forming toxin). In some embodiments, the agent (e.g., a protein encoded by a nucleic acid) can be a pro-survival protein. In some embodiments, the agent is a modulator of the immune system. The agent can activate an adaptive immune response, and innate immune response, or both. In some embodiments, the nucleic acid encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The protein can comprise a CRE recombinase, GCaMP, a cell therapy component, a knock-down gene therapy component, a cell-surface exposed epitope, or any combination thereof. In some embodiments, the protein comprises CFTR, GDE, OTOF, DYSF, MYO7A, ABCA4, F8, CEP290, CDH23, DMD, and ALMS1.

The agent can comprise a non-protein coding gene, such as an RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components piRNA, double stranded RNA, snRNA, snoRNA, and/or long non-coding RNA (lncRNA). In some embodiments, the RNA agent can comprise non-natural or modified nucleotides (e.g., pseudouridine). In some embodiments, the non-protein coding gene can modulate the expression or the activity of a target gene or gene expression product. For example, the RNAs described herein may be used to inhibit gene expression in a target cell, for example, a cell in the central nervous system (CNS). In some embodiments, inhibition of gene expression refers to an inhibition by at least about 20%, 30%, 40%, 50%, 60%, 70%, 80%, 85%, 90%, 95% and 100%. In some cases, the protein product of the targeted gene is inhibited by at least about 20%, 30%, 40%, 50%, 60%, 70%, 80%, 85%, 90%, 95% and 100%. The gene can be either a wild type gene or a gene with at least one mutation. The targeted protein can be a wild type protein, or a protein with at least one mutation.

Examples of genes encoding therapeutic proteins include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a protein comprising an appropriate version of signal transducer. Examples of DNA or RNA sequences contemplated herein include sequences for a disease-associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder.

Many proteins (e.g., enzymes) are secreted and can exert cross-correction effects. For these, the genetic material can be delivered to brain endothelial cells using an AAV therapeutics to other cell types. For example, production of the secreted Sparcl1/Hevin protein in brain endothelial cells can rescue the thalamocortical synapse loss phenotype of Hevin KO mice (See, Example 1 below). This proof-of-concept supports the brain endothelial cell biofactory model for production of enzymes, antibodies, or other biological therapeutics, providing a novel therapeutic approach for diseases like lysosomal storage disorders.

In some embodiments, the rAAV having a capsid protein comprising one or more targeting peptides disclosed herein can be used to deliver genes to specific cell types in the target environment of a subject. For example, the rAAV can be used for delivering genes to neurons and glia in the nervous system (including PNS, CNS, or both) of a subject (e.g., a mammal). The compositions and methods disclosed herein can be used in, for example, (i) reducing the expression of mutant Huntingtin in patients with Huntington's Disease by, for example, incorporating a Huntingtin-specific microRNA expression cassette within a rAAV genome and packaging the rAAV genome into a variant rAAV (e.g., AAV-X1) for delivery through, for example the vasculature, (ii) delivering a functional copy of the Frataxin gene to patients with Friedreich's ataxia, (iii) restoring expression of an enzyme critical for normal lysosomal function in patients lacking expression of the enzyme due to genetic mutation (e.g., patients with Neimann-Pick disease, mucopolysaccharidosis III, and/or Gaucher's disease), (iv) using the rAAV (e.g., AAV-X1) to generate animal models of disease, or a combination thereof.

The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

In some embodiments, the subject is suffering from an acute condition or injury. The subject in need can be a subject suffering from, at risk to develop, or has suffered from a stroke, traumatic brain injury, epilepsy, or spinal cord injury.

### Pharmaceutical Compositions and Methods of Administration

Also disclosed herein are pharmaceutical compositions comprising one or more of the rAAV viruses disclosed herein and one or more pharmaceutically acceptable carriers. The compositions can also comprise additional ingredients such as diluents, stabilizers, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include methods of delivering an agent to a nervous system of a subject. In some embodiments, the method comprises: providing an AAV vector comprising an AAV capsid protein disclosed herein. In some embodiments, the AAV vector comprises an agent to be delivered to the nervous system. In some embodiments, the method comprises administering the AAV vector to the subject. The composition can be for intravenous administration. The composition can be for systemic administration. The agent can be delivered to endothelial lining of the ventricles in the brain, central canal of the spinal cord, capillaries in the brain, arterioles in the brain, arteries in the brain, or a combination thereof of the subject. The subject can be an adult animal.

Titers of the rAAV to be administered will vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration will vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and pharmacological methods.

The exact dosage can be determined on a drug-by-drug basis, in most cases, some generalizations regarding the dosage can be made. In some embodiments, the rAAV for delivery of an agent to the nervous system (e.g., CNS) of a subject can be administered, for example via injection, to a subject at a dose of between 1×10¹⁰ viral genome (vg) of the recombinant virus per kg of the subject and 2×10¹⁴ vg per kg, for example between 5×10¹¹ vg/kg and 5×10¹² vg/kg. In some embodiments, the dose of the rAAV administered to the subject is no more than 2×10¹⁴ vg per kg. In some embodiments, the dose of the rAAV administered to the subject is no more than 5×10¹² vg per kg. In some embodiments, the dose of the rAAV administered to the subject is no more than 5×10¹¹ vg per kg.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure is administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or to vg or viral genomes per kg or into total viral genomes administered by one of skill in the art.

In some embodiments, a dose of the pharmaceutical composition comprises a concentration of infectious particles of at least or about 10⁷, 10⁸, 10⁹, 10¹⁰, 10¹¹, 10¹², 10¹³, 10¹⁴, 10¹¹, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The administration can be a systemic administration. The administration can be an intravenous administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. In some embodiments, the recombinant virus is administered to the subject by systematic transduction. In some embodiments, the recombinant virus is administered to the subject by intramuscular injection. In some embodiments, the rAAV is administered to the subject by the parenteral route (e.g., by intravenous, intramuscular or subcutaneous injection), by surface scarification or by inoculation into a body cavity of the subject. Route(s) of administration and serotype(s) of AAV components of the rAAV virus can be readily determined by one skilled in the art taking into express the protein of interest. In some embodiments, it can be advantageous to administer the rAAV via intravenous administration. The variant AAV provided herein can advantageously provide for intravenous administration of vectors with enhanced tropisms for CNS.

In some embodiments, the subject is a primate and the agent is delivered to the endothelial cells and/or neurons of the nervous system. The nervous system can be the central nervous system (CNS). The agent can be delivered to the endothelial cells of the nervous system of the subject at least 1.5-fold, 2-fold, or 3-fold more efficiently than the delivery of the agent to the neurons of the nervous system. In some embodiments, the agent is delivered to the endothelial cells of the nervous system of the subject more than 3-fold more efficiently (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the neurons of the nervous system.

Disclosed herein include methods of delivering an agent to a cell. In some embodiments, the method comprises: contacting an AAV vector comprising an AAV capsid protein disclosed herein with the cell. In some embodiments, the AAV vector comprises an agent to be delivered to the nervous system. In some embodiments, the cell is an endothelial cell or a neuron. In some embodiments, contacting the AAV vector with the cell occurs in vitro, in vivo or ex vivo. The cell can be present in a tissue, an organ, or a subject. The cell can be a brain endothelial cell, a neuron, a cell in the capillaries in the brain, a cell in the arterioles of the brain, a cell in the arteries in the brain, a cell in the brain vasculature, or a combination thereof.

The AAV vector can be an AAV9 vector, or a variant thereof. In some embodiments, the AAV vector is a vector selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

The nucleic acid can comprise one or more miRNA-122 (miR-122) binding sites. In some embodiments, at least one of the one or more miR-122 binding sites is located in the 3′ UTR of the nucleic acid.

The variant AAV capsid can comprise tropism for a target tissue or a target cell. The target tissue or the target cell can comprise a tissue or a cell of a central nervous system (CNS). The target cell can be a neuronal cell, a neural stem cell, an astrocytes, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell. Schwann cell, glial cell, astroblast, astrocyte, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, foam cell, histiocyte, luteal cell, lymphocytic stem cell, lymphoid cell, lymphoid stem cell, macroglial cell, mast cell, medulloblast, megakaryoblast, megakaryocyte, metamyelocyte, monoblast, monocyte, myoblast, myocyte, muscle cell, smooth muscle cell, myelocyte, myeloid cell, myeloid stem cell, myoblast, myoepithelial cell, myofibrobast, neuroblast, neuroepithelial cell, neuron, pericyte, peripheral blood mononuclear cell, pinealocyte, pituicyte, plasma cell, platelet, reticulocyte, somatotroph, stem cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

Actual administration of the rAAV can be accomplished by using any physical method that will transport the rAAV into the target tissue of the subject. For example, the rAAV disclosed herein can advantageously be administered intravenously for delivery to the CNS. As disclosed herein, capsid proteins of the rAAV can be modified so that the rAAV is targeted to a particular target environment of interest such as central nervous system, and to enhance tropism to the target environment of interest (e.g., CNS tropism). Pharmaceutical compositions can be prepared, for example, as injectable formulations.

The recombinant virus to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the rAAV have been established for at least some condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

[symbol]

subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with CNS, which includes but is not limited to Krabbe disease, Sandhoff disease, Tay-Sachs, Gaucher disease (Type I, II, or 111), Niemann-Pick disease (NPC1 or NPC2 deficiency), Hurler syndrome, Pompe disease, Batten disease, or any combination thereof), chronic pain, or a combination thereof. In some embodiments, the rAAV is administered to the subject during remission of the disease or disorder. In some embodiments, the rAAV is administered prior to the onset of the disease or disorder in the subject. In some embodiments, the rAAV is administered to a subject at a risk of developing the disease or disorder.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving 0-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Disclosed herein, in some embodiments, are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the rAAV compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. In some embodiments, the rAAV disclosed herein can advantageously be administered intravenously for delivery to the CNS.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation variety of dosage forms such as injectable solutions, drug-release capsules, and the like.

Formulations for intranasal administration can comprise a coarse powder comprising the active ingredient and having an average particle size from about 0.2 μm to 500 m. Such formulations are administered in the manner in which snuff is taken, e.g. by rapid inhalation through the nasal passage from a container of the powder held close to the nose. Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions will be within the purview of the skilled artisan having benefit of the present teachings. In some embodiments, however, that the administration of therapeutically-effective amounts of the disclosed compositions may be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In some embodiments, it is advantageous to provide multiple, or successive administrations of the AAV vector compositions, either over a relatively short, or a relatively administration of such compositions. For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in certain embodiments, it may be desirable to administer two or more different AAV vector compositions, either alone, or in combination with one or more other therapeutic drugs to achieve the desired effects of a particular therapy regimen. In various embodiments, the daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The targeting peptides described herein can be used to generate rAAVs with enhanced CNS tropisms with capsid proteins derived from different AAV serotypes (e.g., AAV9 and AAV1). In some embodiments, this can advantageously provide for administration of two or more different AAV vector compositions without inducing immune response in the subject.

The dosing frequency of the rAAV virus can vary. For example, the rAAV virus can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the rAAV virus is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

Disclosed herein are kits comprising compositions disclosed herein. Also disclosed herein are kits for the treatment or prevention of a disease or conditions of the CNS, PNS, or target organ or environment (e.g., CNS). In some instances, the disease or condition is cancer, a pathogen infection, neurological disease, muscular disease, or an immune disorder, such as those described herein. In one embodiment, a kit can include a therapeutic or prophylactic composition containing an effective amount of a composition of a rAAV particle present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, rAAV are provided together with instructions for administering the rAAV to a subject having or at risk of developing the disease or condition. Instructions can generally include information about the use of the composition for the treatment or prevention of the disease or condition.

The kit can include allogenic cells. In some embodiments, a kit includes cells that can comprise a genomic modification. In some embodiments, a kit comprises “off-the-shelf” cells. In some embodiments, a kit includes cells that can be expanded for clinical use. In some embodiments, a kit contains contents for a research purpose.

In some embodiments, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some embodiments, instructions provide procedures for administering the rAAV to the subject alone. In some embodiments, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

### EXAMPLES

Some aspects of the embodiments discussed above are disclosed in further detail in the following examples, which are not in any way intended to limit the scope of the

### Example 1

Brain Endothelial-Specific Cell Targeting in Rodent and Broad CNS Targeting from Rodent to Primate with Engineered Systemic AAVs

This example demonstrates the design of the variant AAVs described herein and illustrates their enhanced CNS tropism in multiple species.

### A Novel AAV Capsid Specifically Targets Brain Endothelial Cells in Mice Following Systemic Administration

Delivering genes to and across the brain vasculature across species remains a critical challenge for addressing neurological diseases. As described herein, adeno-associated virus serotype 9 (AAV9) capsid was evolved into vectors that specifically and efficiently transduce brain endothelial cells following systemic administration in wild-type mice. These AAVs also exhibited superior transduction of the CNS across rodent models (genetically diverse mouse strains and rats), non-human primates (marmosets and rhesus macaques), and ex vivo human brain slices, although, in some embodiments, their endothelial tropism was not conserved across species. In some embodiments, the capsid modifications translated from AAV9 to AAV1, enabling serotype switching for sequential AAV administration. In some embodiments, these endothelial-specific capsids can be used for genetic engineering, transforming the mouse brain vasculature into a functional biofactory for, e.g., the synaptogenic protein Hevin that rescues synaptic deficits in a mouse model.

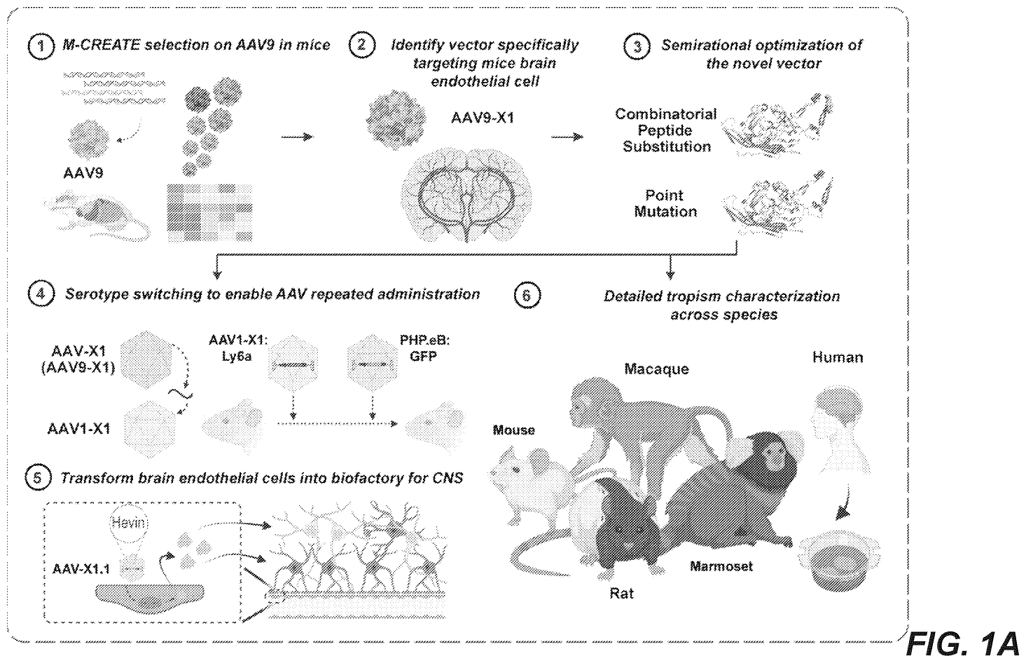

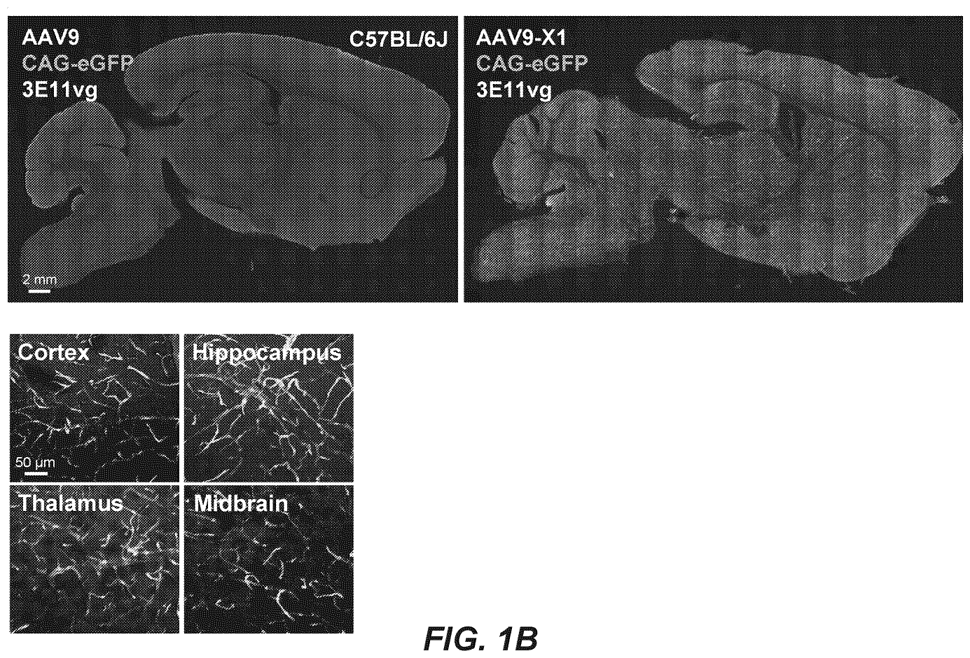

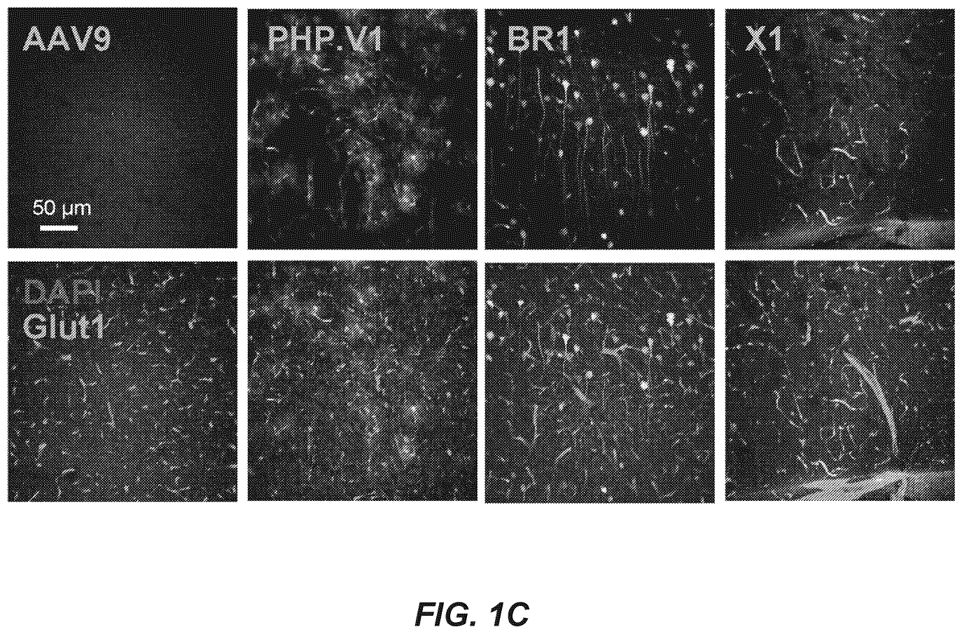

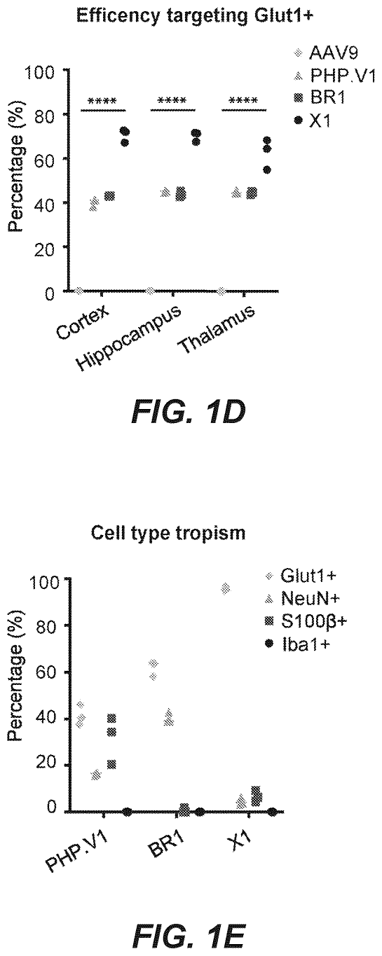

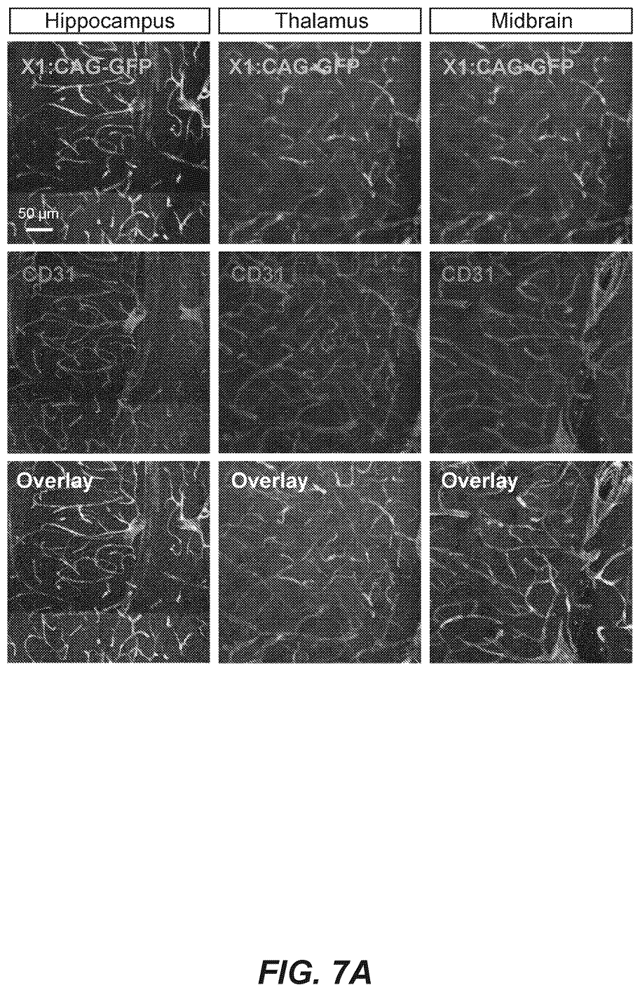

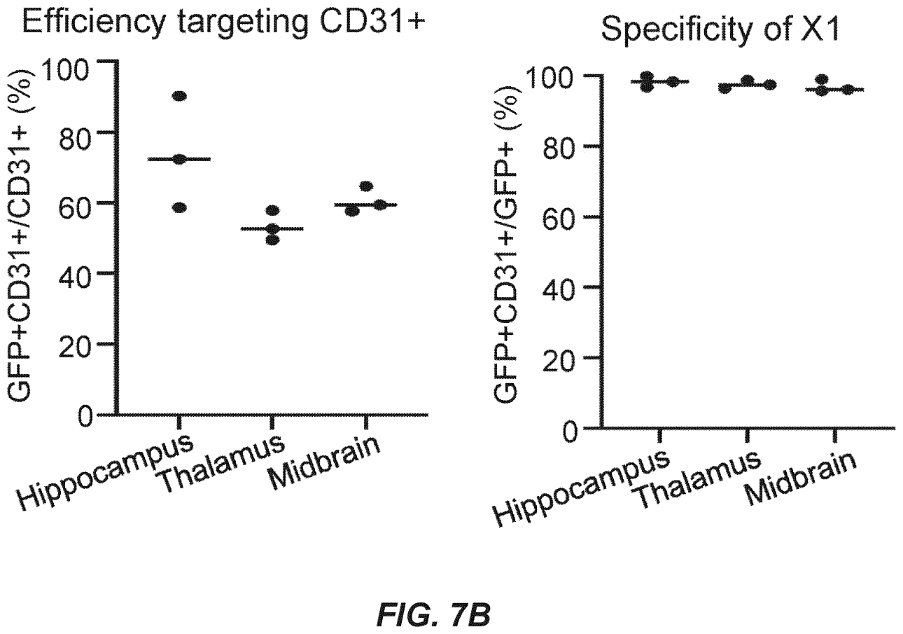

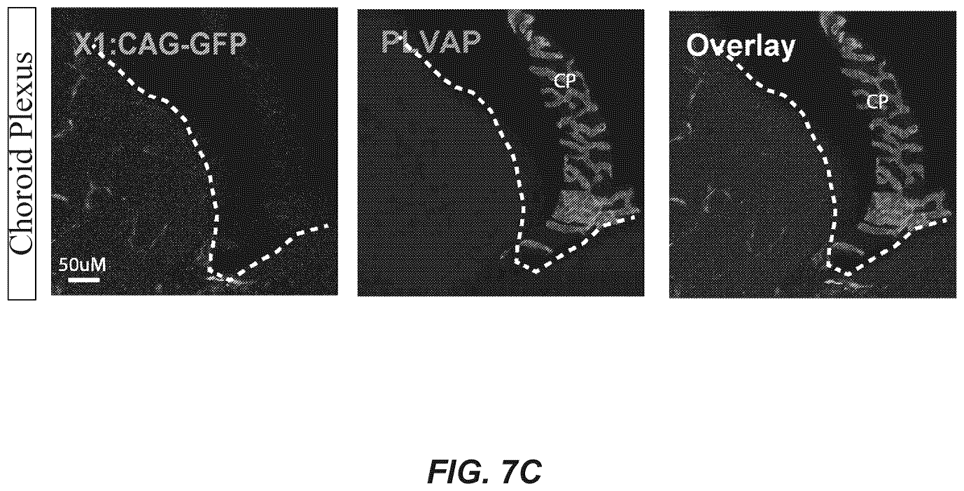

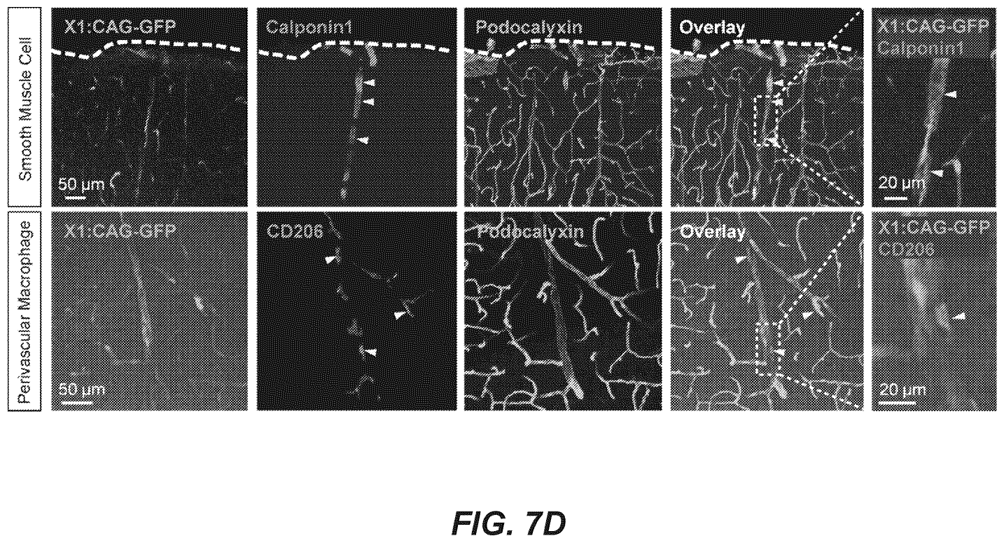

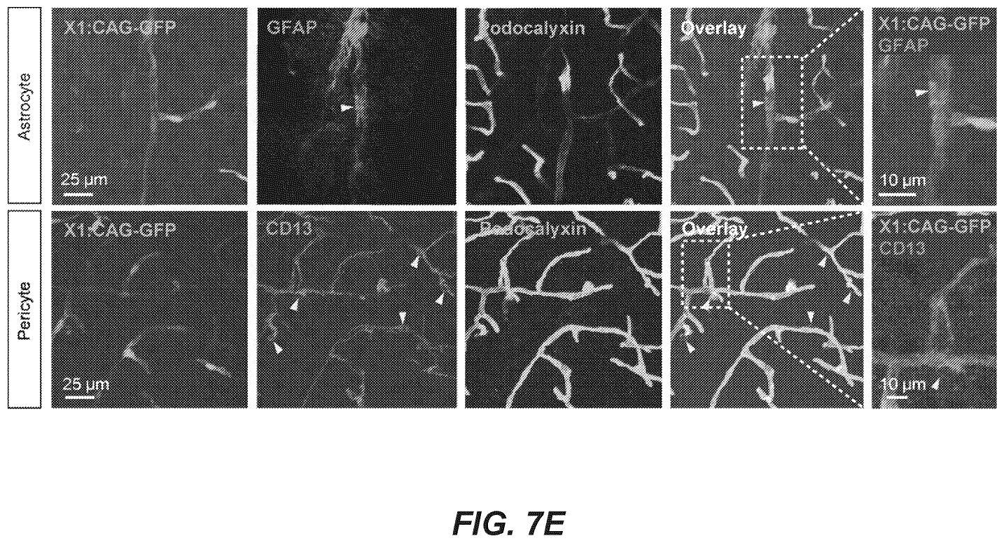

AAV9, which targets the CNS with low efficiency following systemic delivery, was chosen as a starting point for engineering. Randomized 7-mer peptides were inserted between positions 588 and 589 of AAV9 VP1 protein and the resulting virus library was injected intravenously into transgenic mice expressing Cre from either the Tek (targeting endothelial cells) or Synapsin I (Syn, targeting neuronal cells) promoter. After 2 rounds of M-CREATE selection, a variant was identified, named AAV-X1, which was among the most enriched in Tek-Cre mice while being negatively enriched in Syn-Cre mice, indicating a non-neuronal tropism (FIG. 1A). To characterize the transduction profile of AAV-X1 in vivo it was packaged with a single-stranded (ss) AAV genome carrying a strong ubiquitous promoter, CAG, driving the expression of an eGFP reporter. Three weeks after IV administration to C57/BL/6J mice, specific and efficient targeting of the vasculature across brain regions was observed (FIG. 1). To compare the vascular targeting of AAV-X1 to that of previously engineered capsids, the experiment was repeated along with AAV9, AAV-PHP.V1, and AAV-BR1. After 3 weeks of expression, AAV-X1 exhibited higher efficiency and higher specificity in targeting brain endothelial cells compared to all 3 controls (FIG. 1C-FIG. 1E). At a dose of 3×10¹¹ viral across the cortex, hippocampus and thalamus. In comparison, AAV-PHP.V1 and AAV-BR1 transduced ˜40% of Glut1+ cells, while AAV9 transduced ˜1% of the endothelial cells. AAV-X1 also exhibited superior specificity towards vasculature; ˜95% of the cells transduced by AAV-X1 were Glut1+. In contrast, AAV-BR1 and AAV-PHP.V1 transduced Glut1+ cells with much less specificity, with only 60% and 40% of transduced cells being Glut1+, respectively. AAV-BR1 also targeted neurons, while AAV-PHP.V1 also targeted neurons and astrocytes (FIG. 1C-FIG. 1E). Further staining with CD31 or Podocalyxin (endothelial cells), Calponin 1 (smooth muscle cells), CD206 (perivascular macrophages), GFAP (astrocytes) and CD13 (pericytes) confirmed the high efficiency and specificity of AAV-X1 in targeting brain endothelial cells (FIG. 7A-FIG. 7E). Interestingly, the endothelial cells in the choroid plexus (CP) were not transduced by AAV-X1 (FIG. 7C). In summary, the novel vector AAV-X1 not only exhibits significant improvement in targeting the CNS compared to its parent AAV9 but also shows high specificity towards brain endothelial cells.

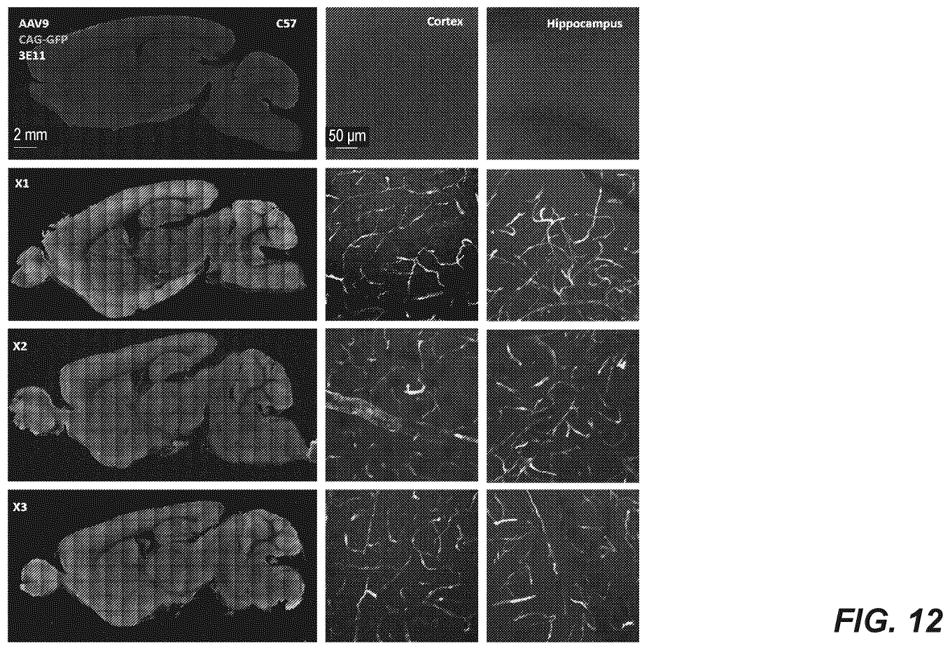

In addition to AAV-X1, variants AAV-X2 and AAV-X3 were also identified, which also showed enhanced tropism for endothelial cells as compared to AAV9 (FIG. 12). VP1 amino acid sequences for AAV-X1, AAVX-2, and AAV-X3 are shown in Table 1.

### Refinement of X1's Peripheral Tropism by Further Engineering Cargo and Capsid

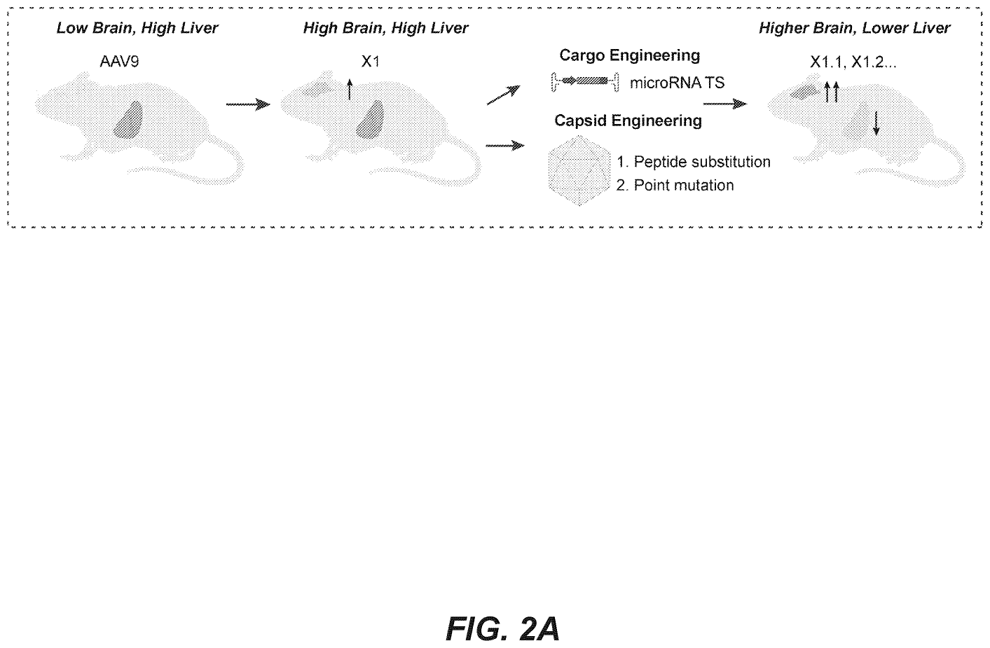

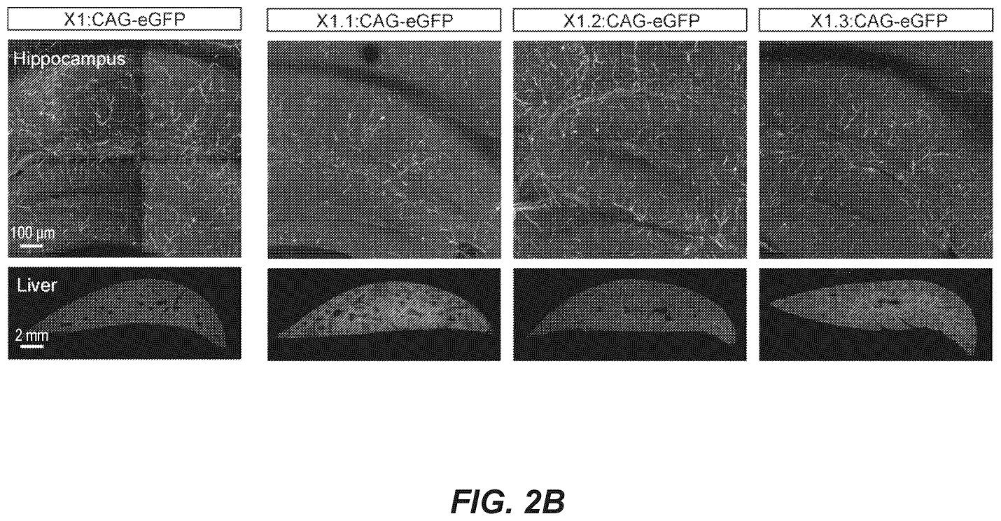

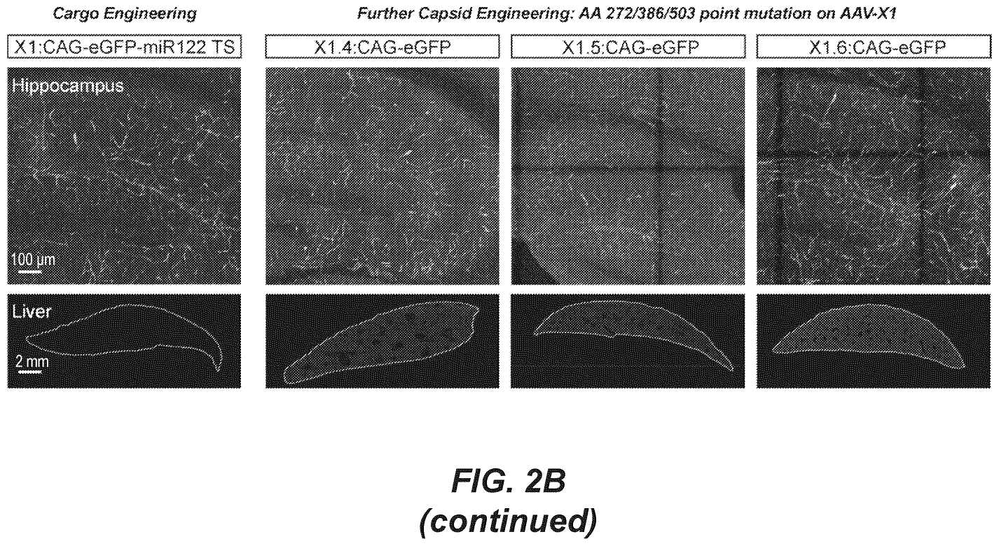

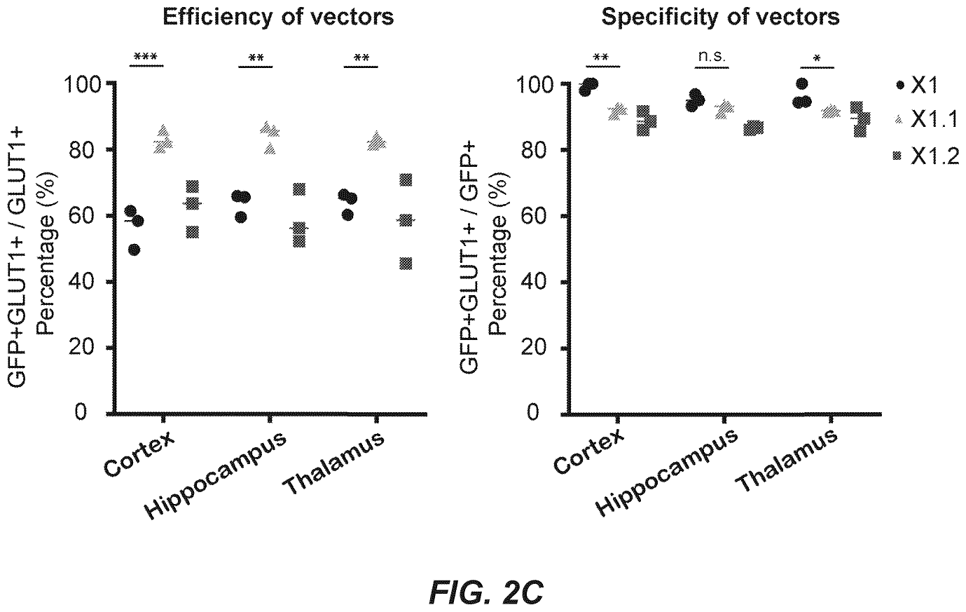

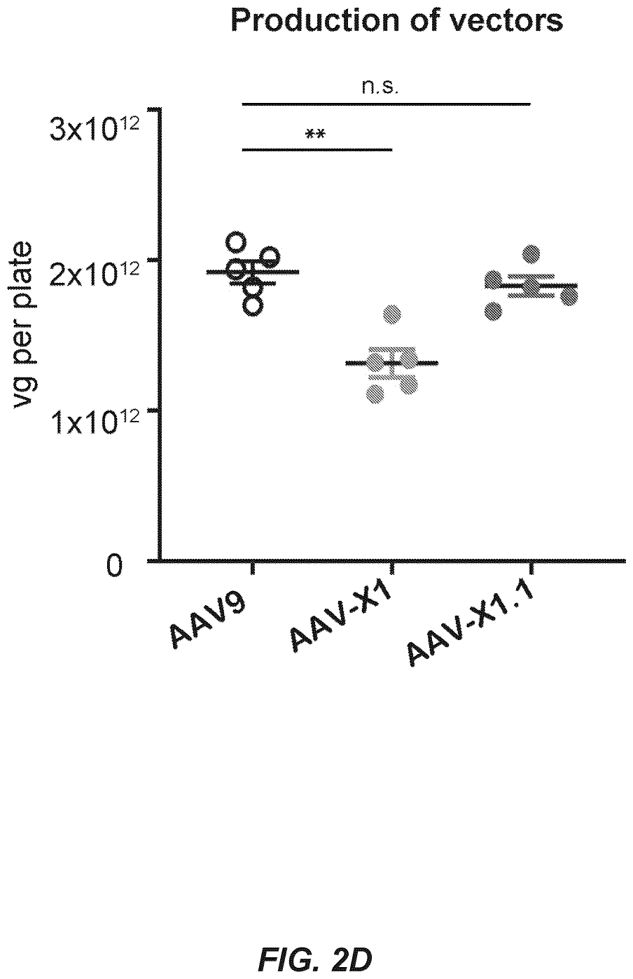

While the novel variant X1 exhibited significant improvement in transducing the CNS endothelium compared to AAV9, in some embodiments, it maintained a similar level of liver transduction. The vector's tropism was further refined, either by incorporating specific cargo elements or by further engineering the capsid (FIG. 2A).

It was studied whether adding a regulatory element to X1's cargo could reduce liver expression. When incorporated into the genome, microRNA target sites induce microRNA-mediated post-transcriptional transgene silencing. MicroRNA-122 (miR-122) is highly specific to the liver and incorporation of its target site has been shown to reduce AAV expression in the liver. 3 copies of miR-122's target site (TS) were cloned into a CAG-eGFP genome and packaged into the X1 vector. Three weeks after IV delivery of X1: CAG-eGFP-miR122TS, a large reduction in liver expression was observed, indicating that miR-122 TS can be incorporated into X1 viral genomes for liver de-targeting (FIG. 2B).

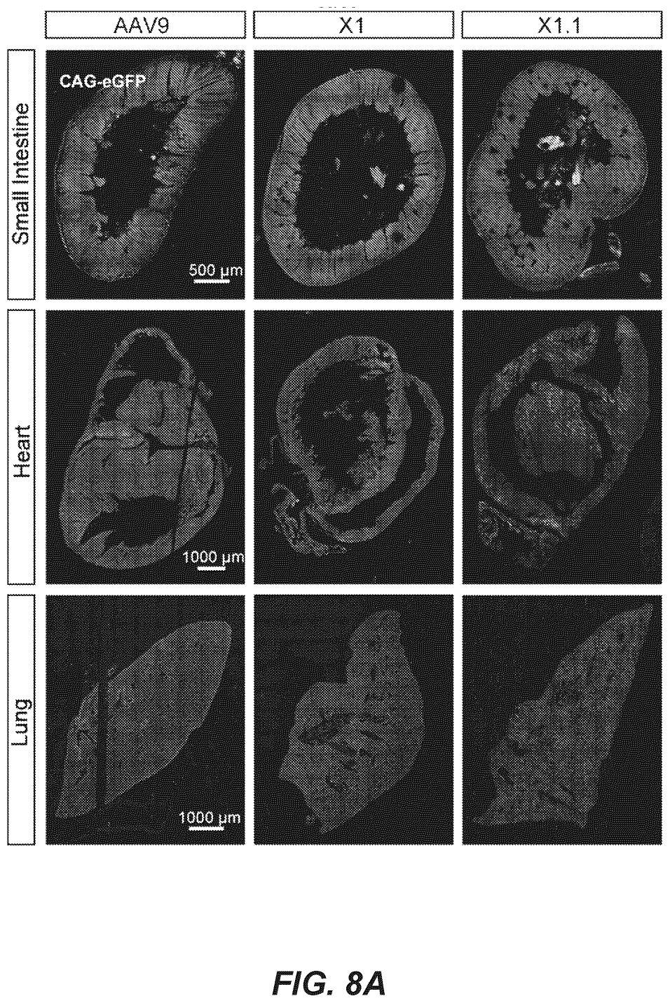

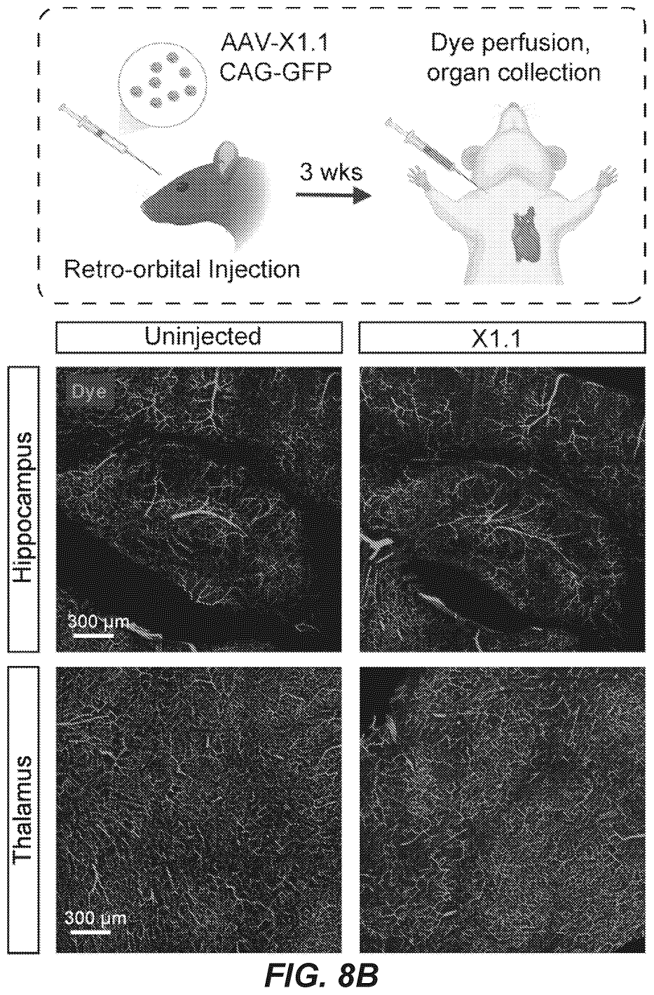

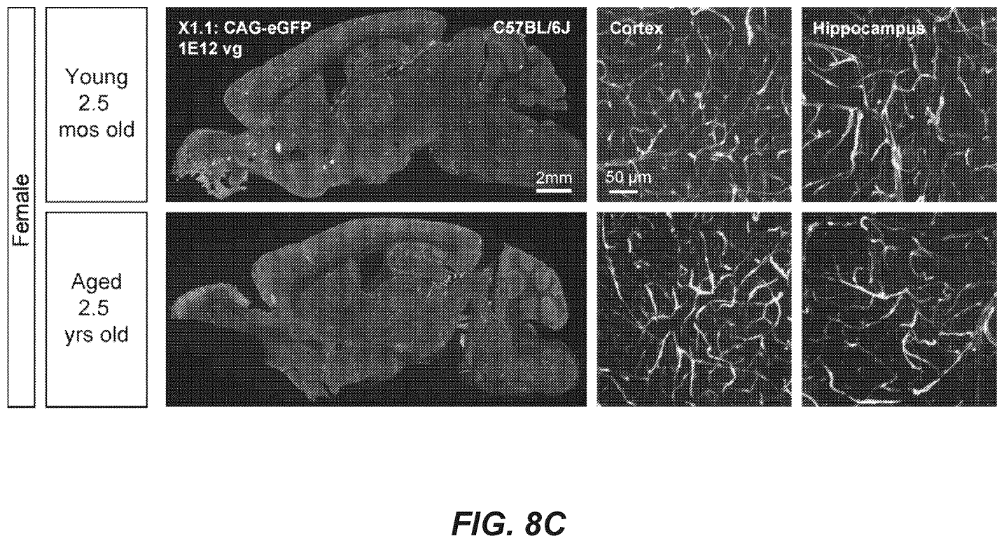

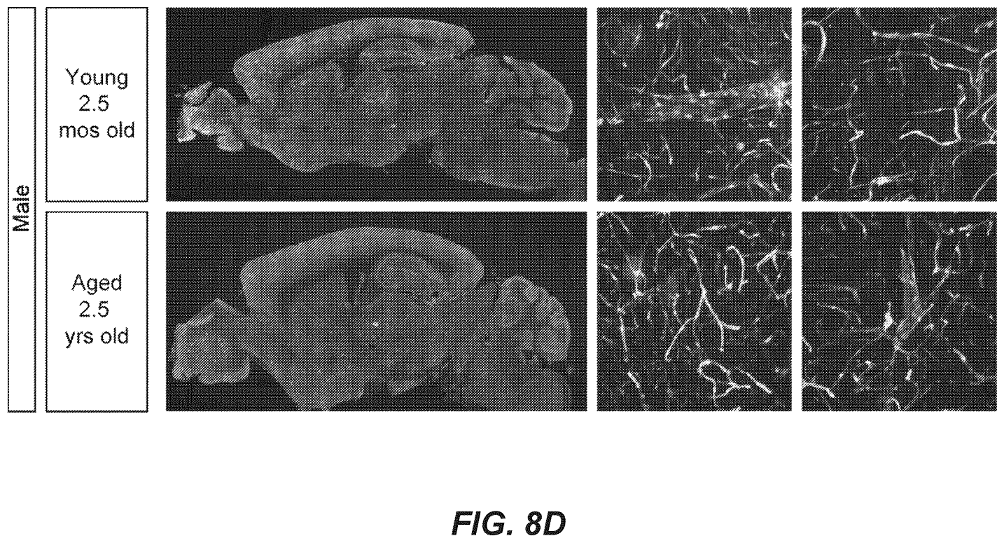

While cargo engineering reduced transgene expression in the liver, the AAV capsid still entered the liver and might, in some embodiments, trigger an immune response. Therefore, it was next studied whether further engineering of the X1 capsid could achieve similar liver de-targeting, and potentially further boost CNS transduction. Without being bound by any particular theory, structural information and previous engineering efforts suggest that the of 452-458 of PHP.eB was found to further boost that capsid's CNS targeting ability and enable potent transduction of the mouse and marmoset CNS while de-targeting the AAV from the liver. Three 7-mer peptides identified from that PHP.eB 455 loop selection (Table 2) were substituted into X1, creating X1.1, X1.2, and X1.3 (See, Table 1 for protein sequences of VP1 protein for X1.1, X1.2, and X1.3). Compared to X1, X1.1 showed further improvement in targeting the CNS, transducing ˜82-85% of Glut1+ cells across brain regions (FIG. 2B-FIG. 2C). X1.1 also maintained high specificity in targeting the brain vasculature; ˜90-92% of transduced cells were Glut1+(FIG. 2C, right). X1.1 showed a similar expression pattern as X1 in peripheral organs such as the heart and lung (FIG. 8A) and was capable of packaging viral genomes with similar efficiency to AAV9 (FIG. 2D).

Given X1.1's high transduction efficiency of brain endothelial cells, a dye perfusion experiment was performed to evaluate whether AAV transduction may compromise BBB integrity. No increased leakage in the BBB compared to un-injected mice was detected (FIG. 8B). Having seen that X1 can accommodate additional capsid changes, it was next investigated whether the vector's tropism could be optimized by mutating sites known to mediate receptor interactions and affect tropism. N272 and W503 have been shown to be required for AAV9's binding to galactose, and mutation of W503 has been shown to reduce the liver transduction of AAV9. Therefore, N272 or W503 of X1 were mutated to alanine, yielding X1.4 and X1.5, respectively. S386 of AAV9 and Q386 of AAV1 have also been shown to mediate receptor interaction, so S386 of X1 was mutated to alanine, yielding X1.6 (See, Table 1 for protein sequences of VP1 protein for X1.4, X1.5, and X1.6). Following IV delivery of these vectors in mice, X1.4 and X1.5 showed reduced liver transduction while maintaining their brain-endothelial tropism (FIG. 2B, bottom). To study the novel vectors in different physiological contexts, the X1.1 vector was tested in young (2.5 months old) versus aged mice (2.5 years old) and no obvious difference was observed in CNS transduction after 3 weeks expression. No obvious sex difference in its CNS tropism was observed (FIG. 8C-FIG. 8D).

### X1 Vectors Transduce Brain Endothelial Cells Across Diverse Mouse Strains and Rats in a Ly6a-Independent Manner

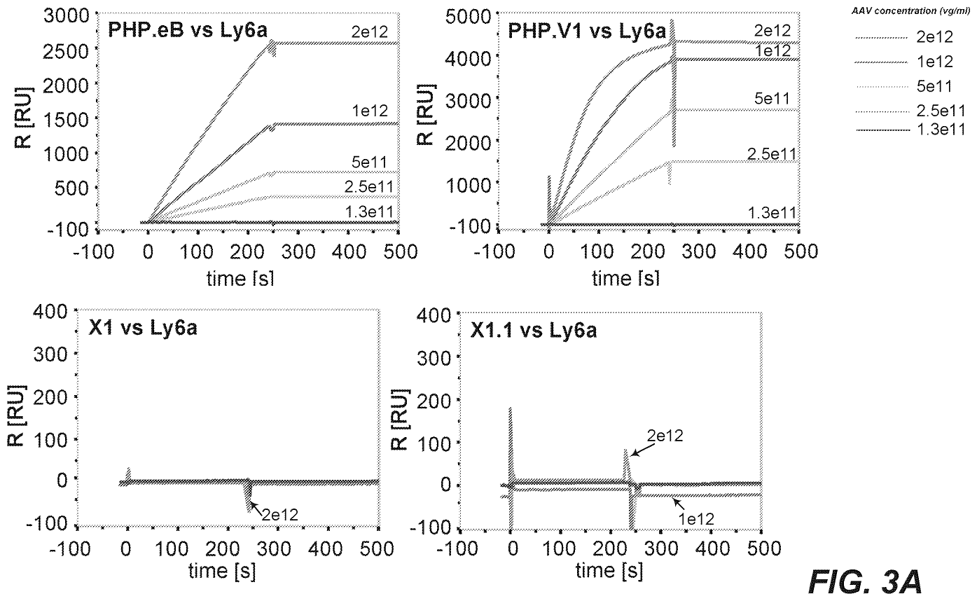

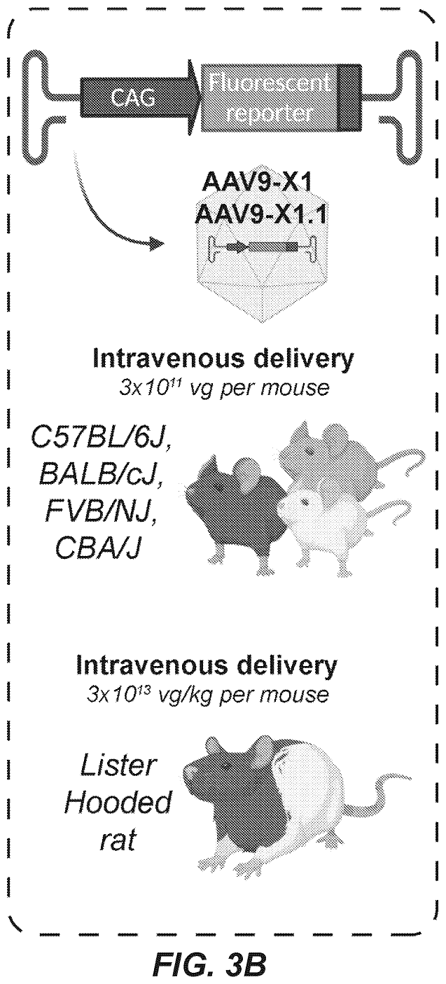

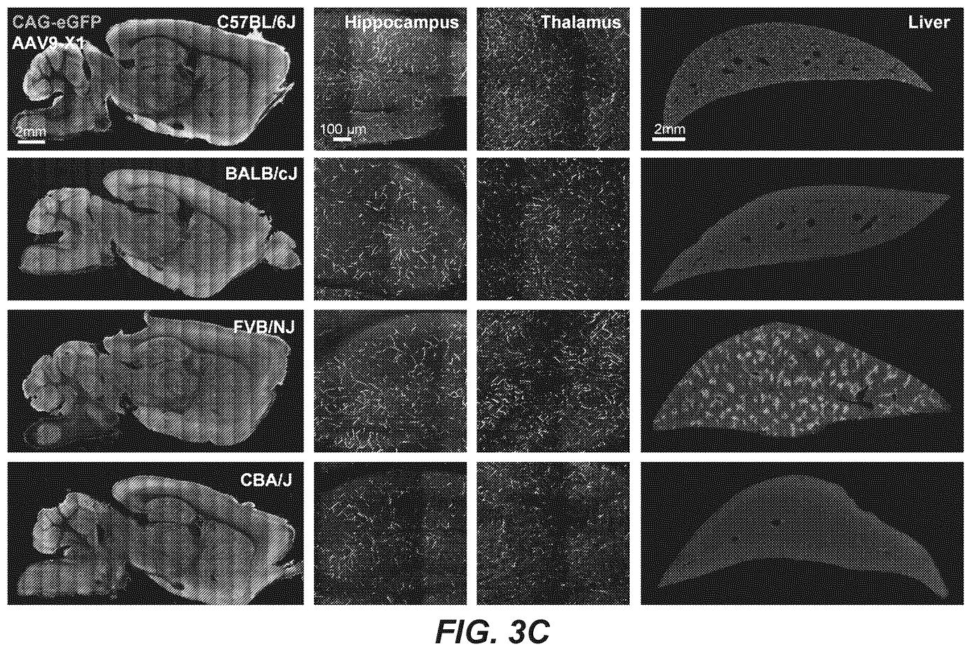

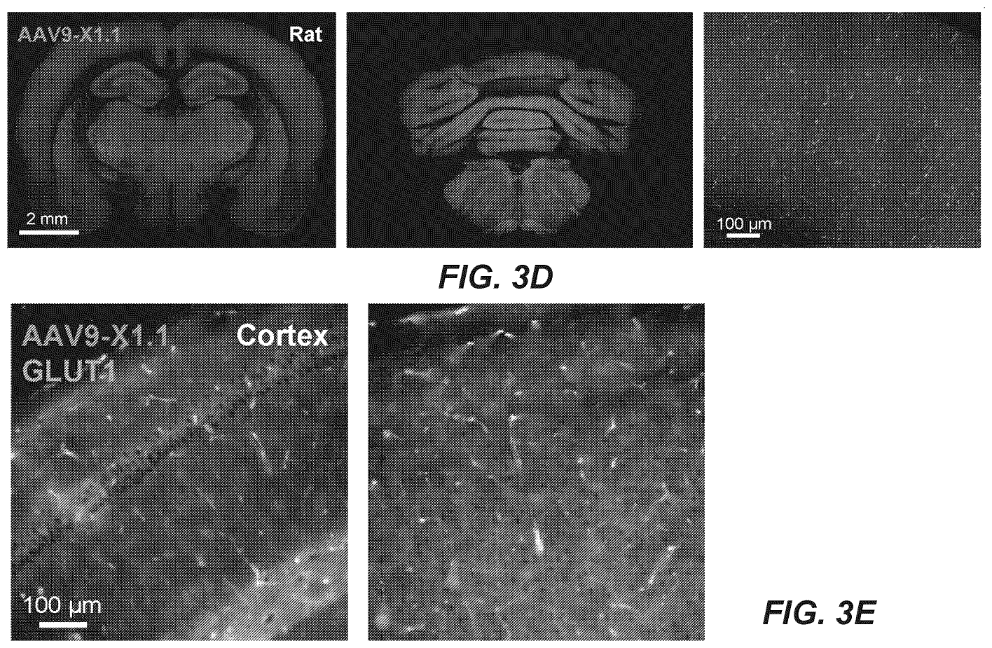

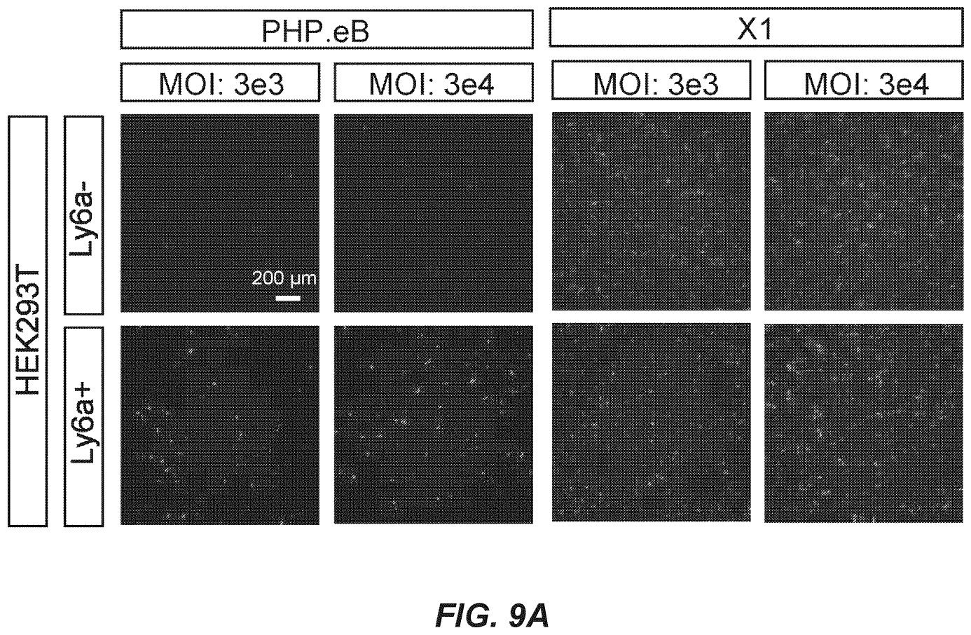

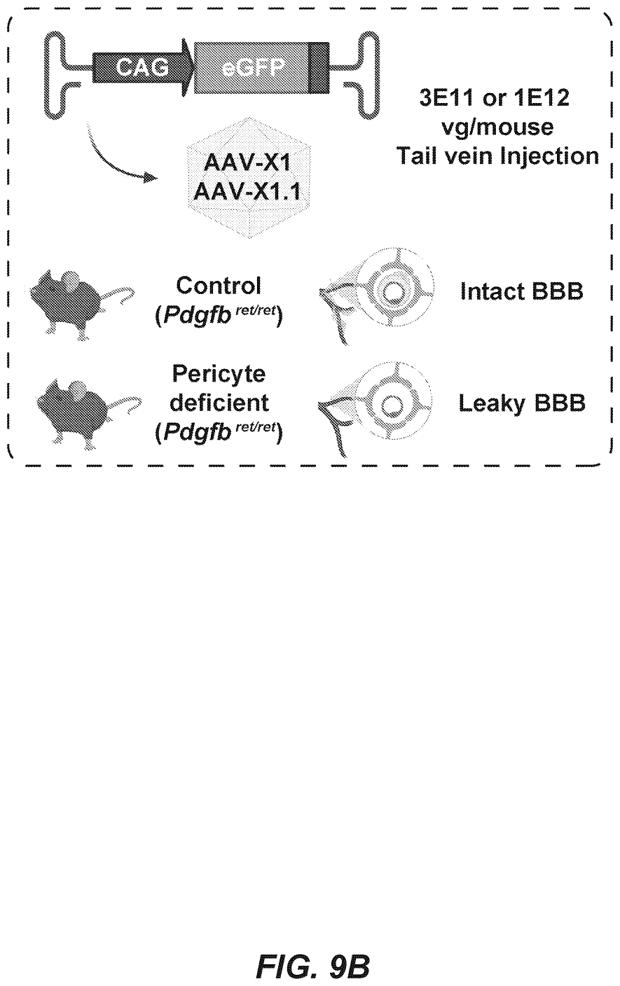

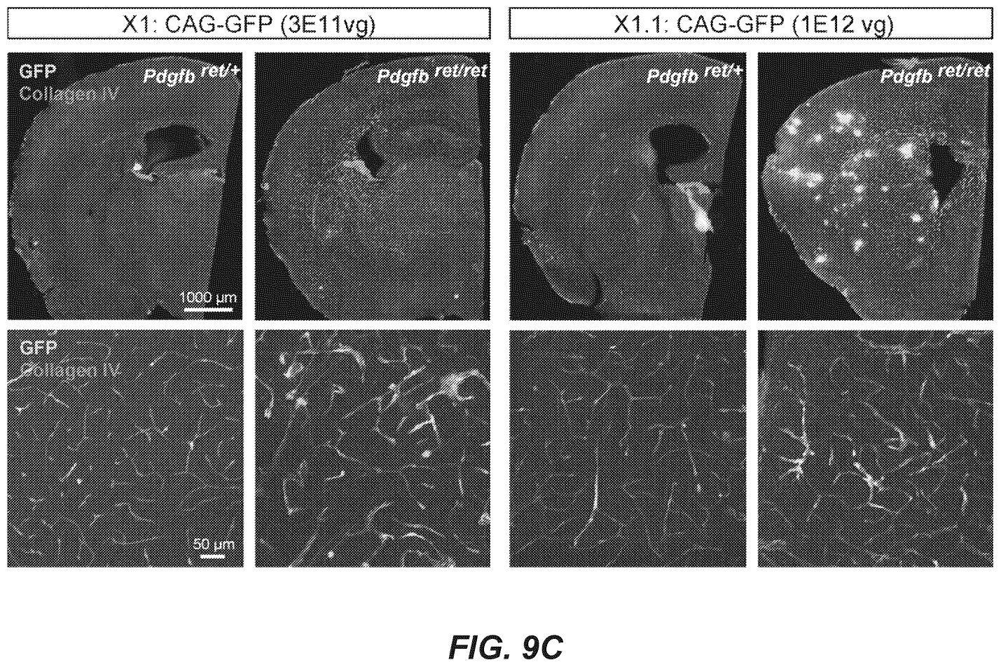

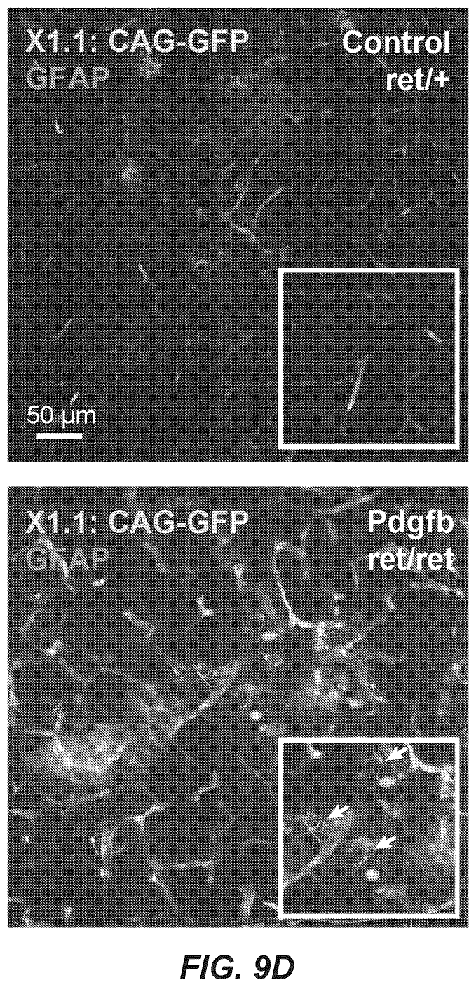

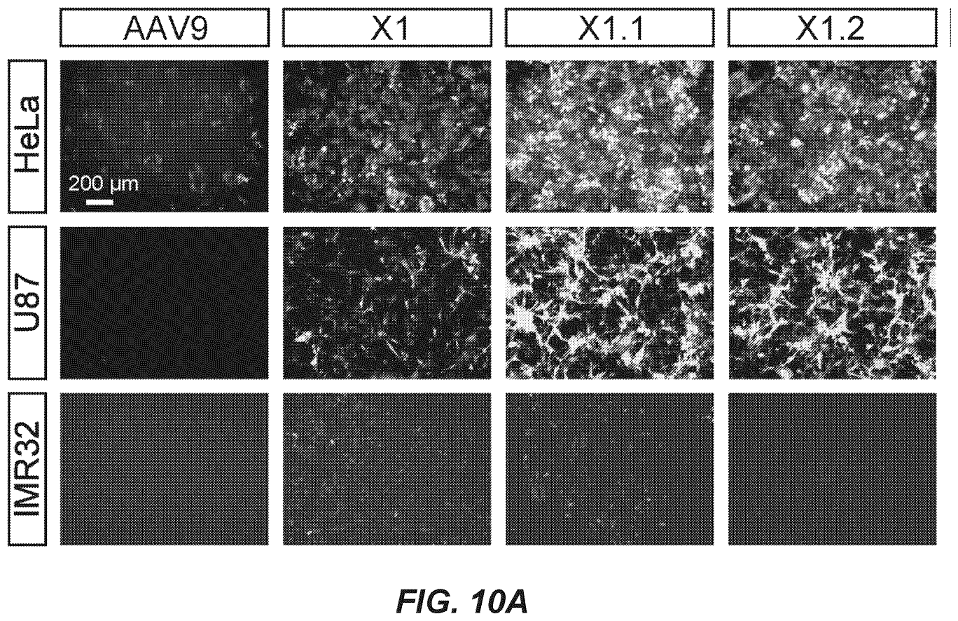

AAV-PHP.eB has been shown to rely on improved binding to Ly6a for its increased CNS tropism, and polymorphism of Ly6a across mouse strains contributes to its strain-specific phenotype. Therefore, it was studied whether the improved CNS targeting of X1 and its further-engineered versions is also dependent on Ly6a binding. It was observed that transient overexpression of Ly6a in HEK cells boosted PHP.eB transduction but had no significant effect on X1 transduction (FIG. 9A). Surface Plasmon Resonance (SPR) experiments exhibited strong binding to Ly6a, explaining the strain-specific phenotype of PHP.V1 (FIG. 3A). However, neither X1 nor X1.1 bound Ly6a (FIG. 3A). These results indicate that X1 and its derivatives utilize a novel mechanism of cell targeting.

The Ly6a-independence of the X1 vectors prompted investigation of their translatability across mice strains. Systemic delivery of X1 capsid packaged with ssAAV:CAG-GFP in BALB/cJ, CBA/J, and FVB/NJ mouse strains resulted in efficient transduction of endothelial cells across brain regions (FIG. 3B-FIG. 3C). DBA/2 mouse strain also shows efficient transduction in the brain with X1. Interestingly, though, different expression patterns were observed in the liver across the strains, with minimal liver transduction in BALB/c and CBA/J strains (FIG. 3C). The new variants' transduction profile in rats was investigated next. Following intravenous delivery of X1.1 capsid packaging ssAAV:CAG-tdTomato in adult Lister Hooded rats, robust expression in the brain was observed (FIG. 3D). X1.1-induced expression overlapped with Glut1+, indicating that its tropism towards endothelial cells is conserved (FIG. 3E).

To study tropism when the BBB is not intact, the vectors were tested in pericyte-deficient mice (FIG. 9B). Increased transduction of other cell types compared to control mice was observed (FIG. 9C). Interestingly, astrocytes with end-feet on the brain vasculature were transduced in pericyte-deficient mice but not in control mice (FIG. 9D), indicating that the novel vectors' specificity towards endothelial cells can be, in some embodiments, dependent on the status of the BBB.

Serotype Switching of X1 from AAV9 into AAV1 Enables Repeat Administration of AAV, Increasing Permeability of the Mouse CNS to AAVs

Repeat administration of AAV is desired for certain applications, however, neutralizing antibodies induced by an initial administration prevent the subsequent delivery of similar AAVs. One potential solution is to switch AAV serotypes in sequential administrations, but most of the novel AAVs engineered to target the CNS, including PHP.B and AAV-X1, are based on AAV9. To overcome this potential hurdle and enable repeat administration, the X1 peptide was transferred to another AAV serotype to recreate the engineered AAV in another natural serotype.

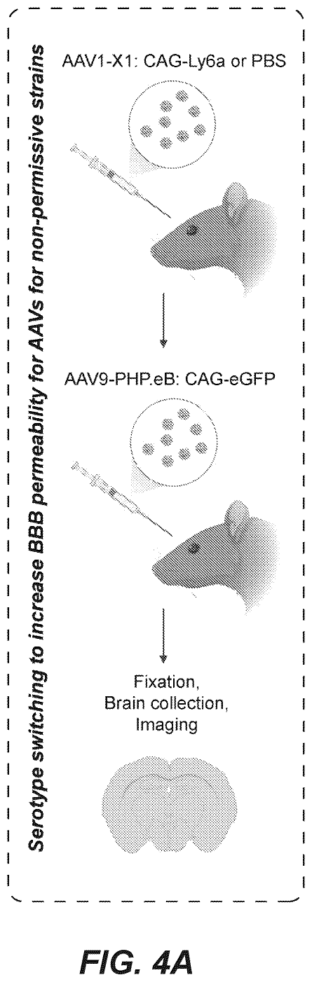

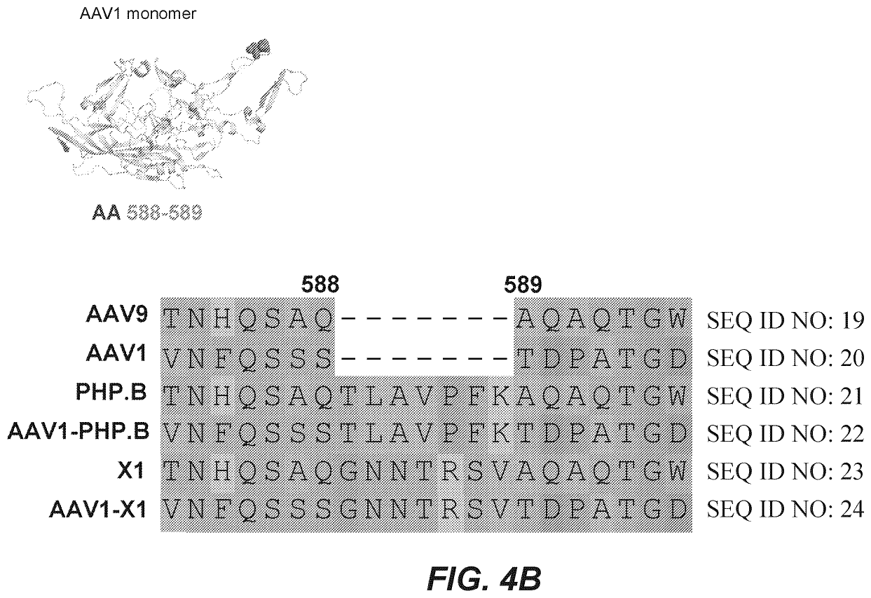

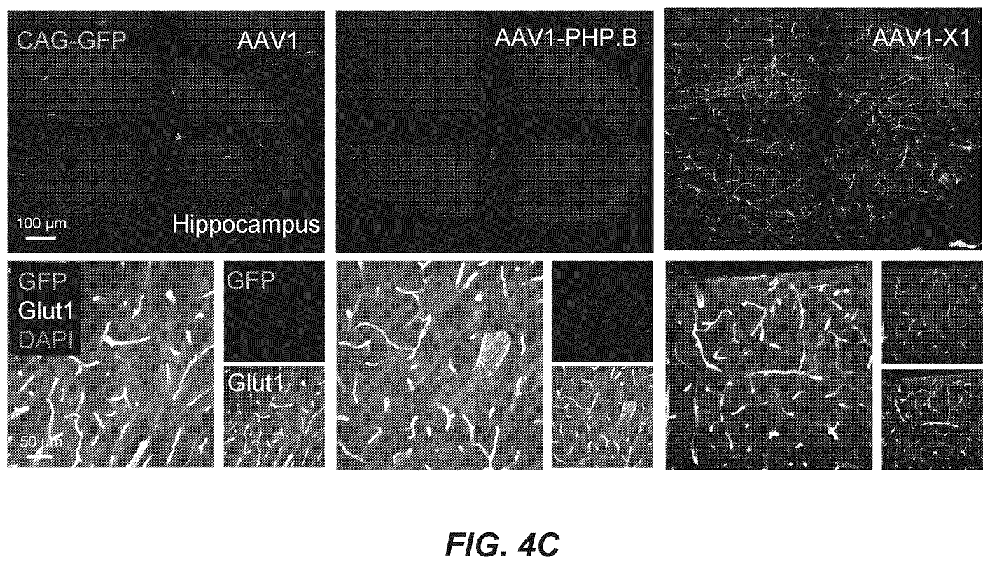

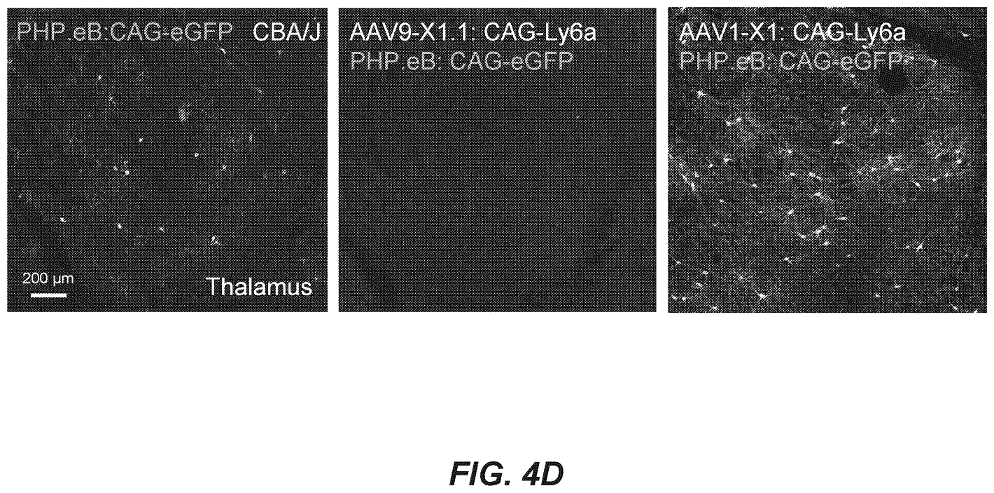

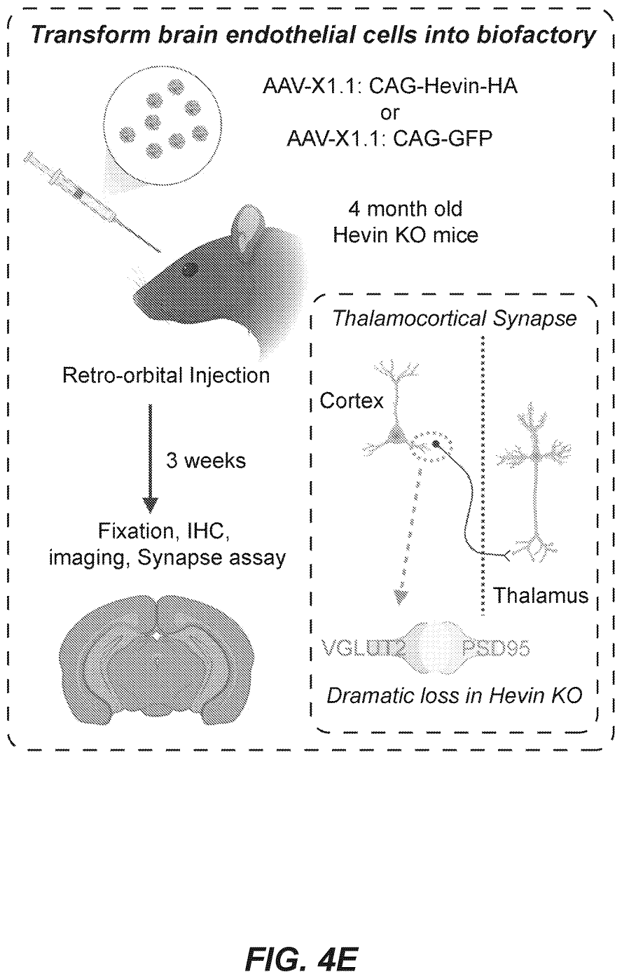

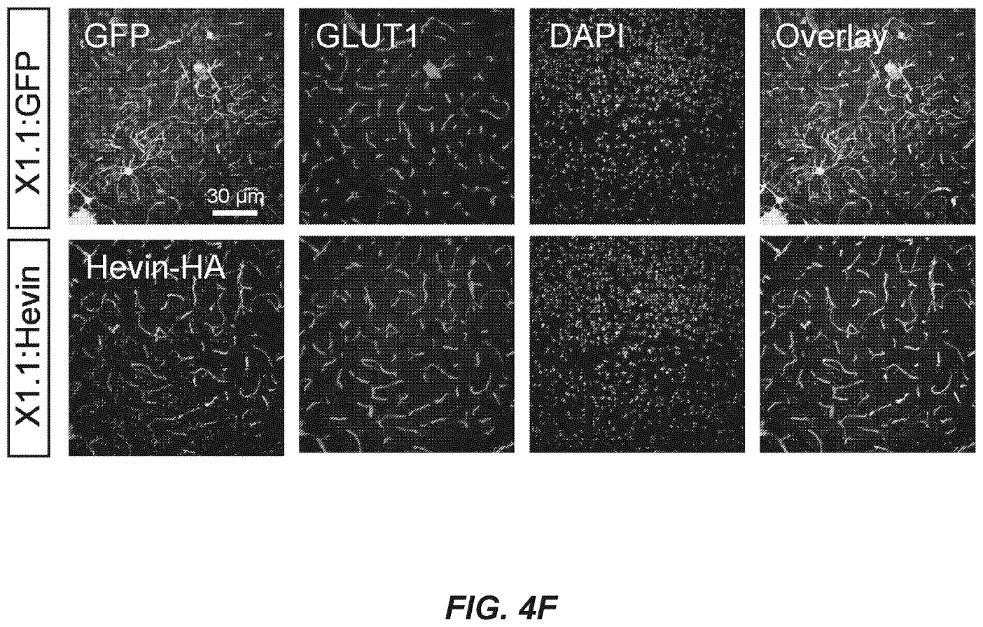

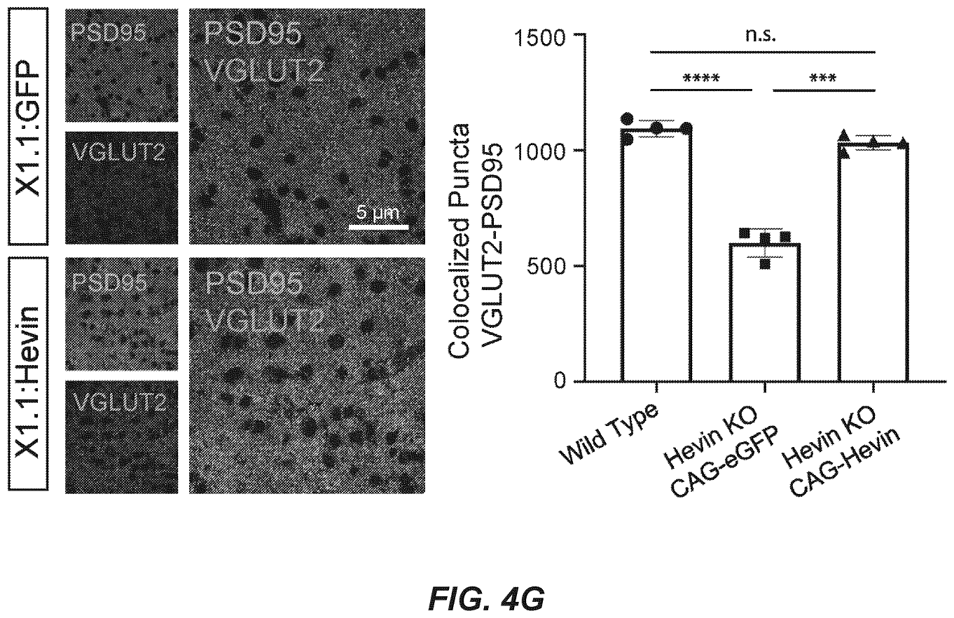

Mixed results have been reported for previous attempts to directly transfer the PHP.B or PHP.eB peptide to natural serotypes, usually AAV1 due to its CNS tropism. The 7-mer peptide of PHP.B was inserted into the AAV1 capsid, creating the hybrid AAV1-PHP.B (FIG. 4B). Intravenous injection of adult mice with AAV1-PHP.B packaging CAG-GFP showed no obvious improvement in brain transduction compared to AAV1. However, AAV1-X1, that of X1, efficiently and specifically targeting Glut1+ endothelial cells (FIG. 4C). This result indicates that X1's phenotype, in contrast to other engineered AAV vectors, can be transferred to other natural serotypes by transferring the 7-mer peptide.

To test AAV1-X1's potential to enable repeat administration, the virus was utilized for Ly6a supplementation. Polymorphisms in Ly6a in certain mouse strains such as CBA/J and BALB/cJ greatly reduce the CNS permeability of PHP.eB. The brain endothelial cell tropism of X1 and AAV1-X1 prompted their utilization for expression of C57BL/6J-like Ly6a in the BBB of these non-permissive strains, thereby increasing the permeability of those animals' BBB for AAV9-based PHP.eB when it is subsequently administered. AAV1-X1 or X1.1 capsid packaged with Ly6a was intravenously delivered into adult CBA/J mice. After three weeks of expression, AAV9-PHP.eB packaged with eGFP was injected into the same mice (FIG. 4A, FIG. 4D). Increased GFP expression in the brains of CBA/J mice injected with AAV1-X1:CAG-Ly6a was observed but not in the mice injected with AAV9-X1.1:CAG-Ly6a, indicating that AAV1-X1 enables the subsequent administration of AAV9-PHP.eB and also facilitates the permeability of AAV9-PHP.eB (FIG. 4D). This result indicates that the serotype switching paradigm enabled by AAV1-X1 can provide a solution for AAV re-administration in mice.

The X1.1 Vector can Transform Brain Endothelial Cells into a Biofactory for Secreted Protein Delivery to the Brain

The broad distribution of vasculature across brain regions creates the opportunity to transform endothelial cells into a biofactory for the broad production of therapeutic agents such as secreted proteins with trophic properties for other cell types within the CNS. For secreted proteins, this would remove the production burden from the target cell, which may already reside in a disease state. The AAV vectors disclosed herein transduce brain endothelial cells efficiently and specifically, and thus can be used for such application.

Sparc-like protein 1 (Sparcl1), which is also known as Hevin, is a matricellular secreted protein that is predominantly expressed by astrocytes and a subset of neurons in the CNS. Endothelial cells also express Sparcl1 mRNA; however, protein expression by these cells has not been observed. Downregulation or missense mutations in Hevin have been reported in numerous neurological disorders such as autism, schizophrenia and depression. In the developing mouse visual cortex, Hevin is specifically required for the formation and plasticity of thalamocortical connections. Hevin knockout mice (Hevin KO) display a dramatic loss of Vesicular Glutamate Transporter 2 positive (VgluT2+) thalamocortical synapses both in the first three postnatal weeks and as adults (FIG. 4E).

[symbol]

rescue the deficits observed in Hevin KO mice, a viral vector for Hevin expression was generated using AAV X1.1. Indeed, AAV X1.1 packaging Hevin efficiently transduced brain endothelial cells and drove the expression of Hevin in these cells. To investigate whether Hevin production via endothelial cells would rescue the deficits in thalamocortical synapses observed in Hevin KO mice, both Hevin-HA and eGFP driven by the CAG promoter were retro-orbitally delivered into 4-month-old Hevin KO mice (FIG. 4E). After 3 weeks, robust expression of both transgenes was observed in brain endothelial cells (FIG. 4F). Further, a synapse assay using the presynaptic thalamocortical marker Vglut2 and postsynaptic marker PSD95 in Hevin KO mice showed a significantly higher number of thalamocortical synapses in layer IV of the V1 cortex in the treated group (CAG-Hevin) compared to the control (CAG-eGFP) (FIG. 4G). This result indicates that the Hevin produced and secreted from the brain endothelial cells was able to mimic endogenous Hevin function, rescuing the thalamocortical synapse formation that is severely deficient in Hevin KO mice. This system thus illustrates a new therapeutic strategy for Hevin-related disorders and other similar disorders mediate by secreted or ECM factors.

### The X1 Vector Family Efficiently Transduces Human Brain Endothelial Cells In Vitro

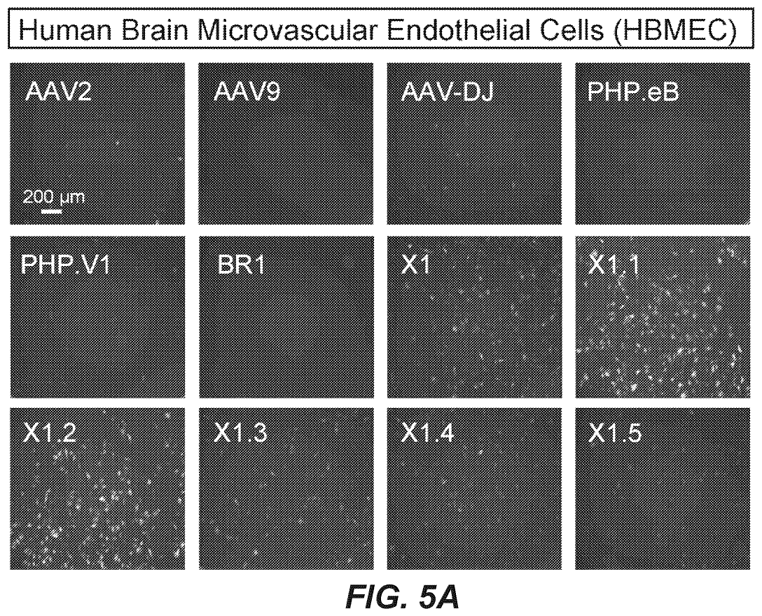

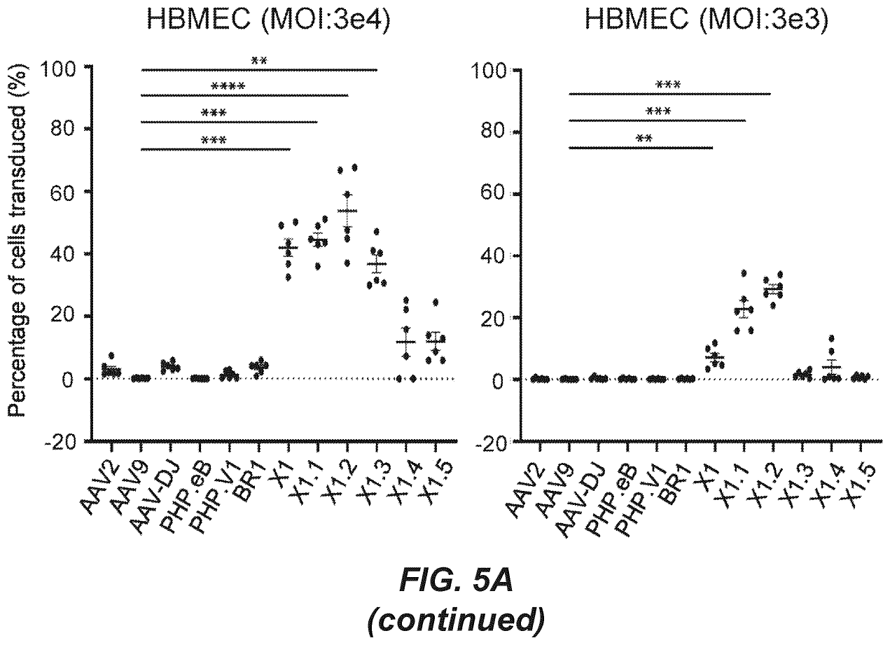

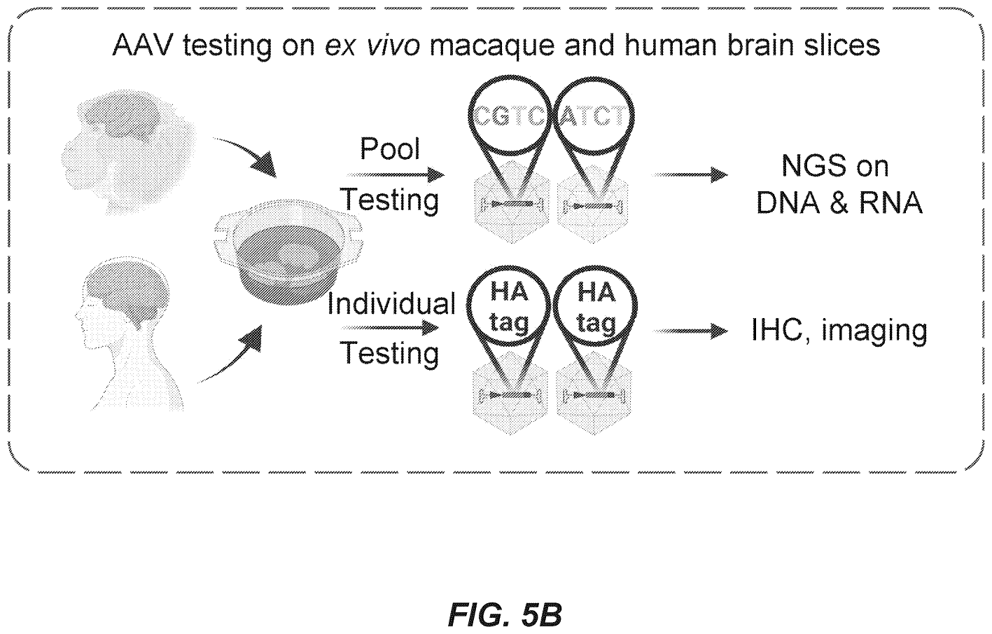

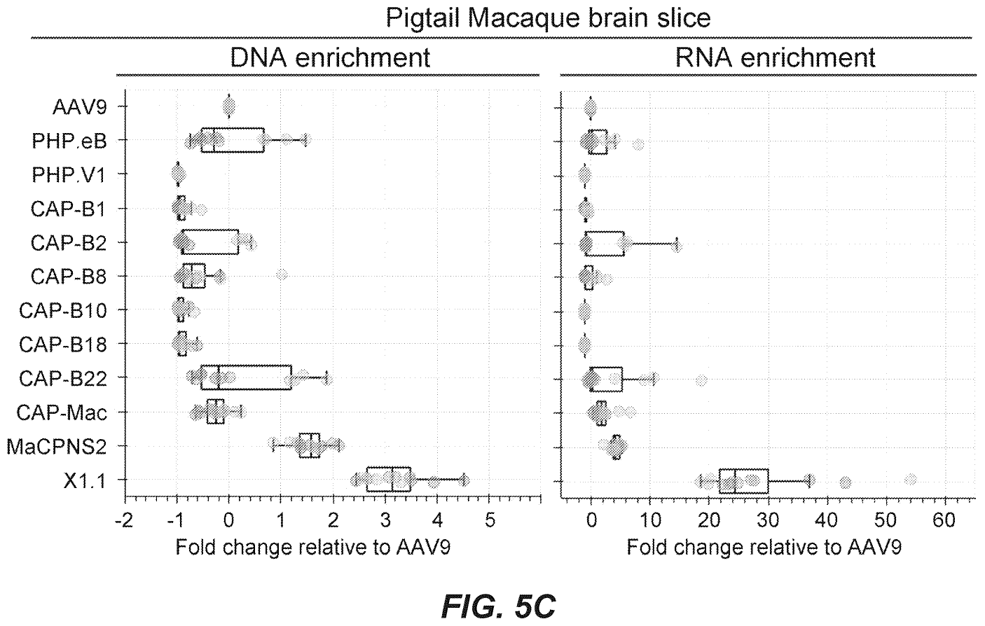

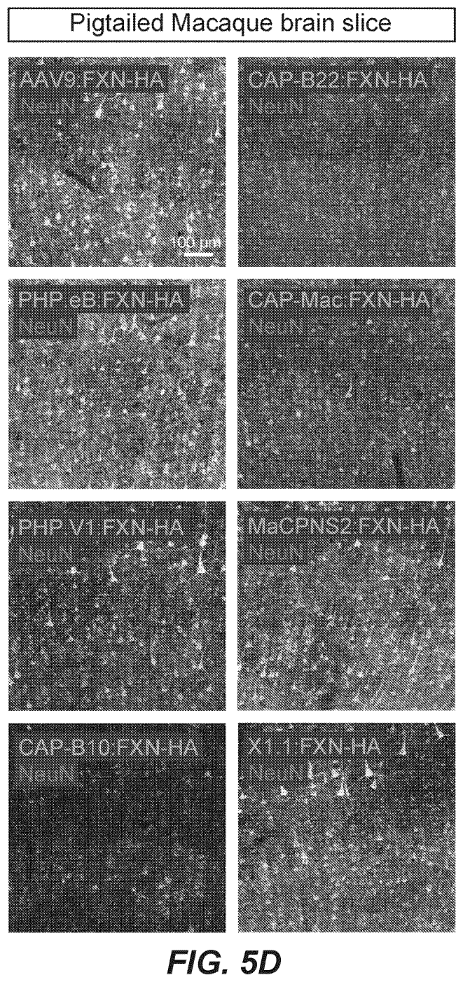

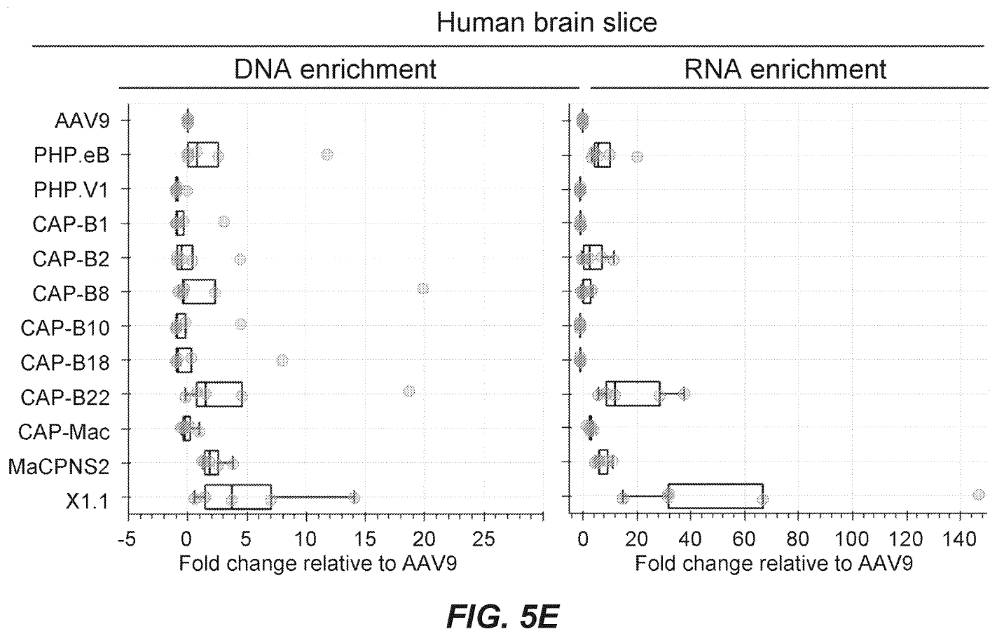

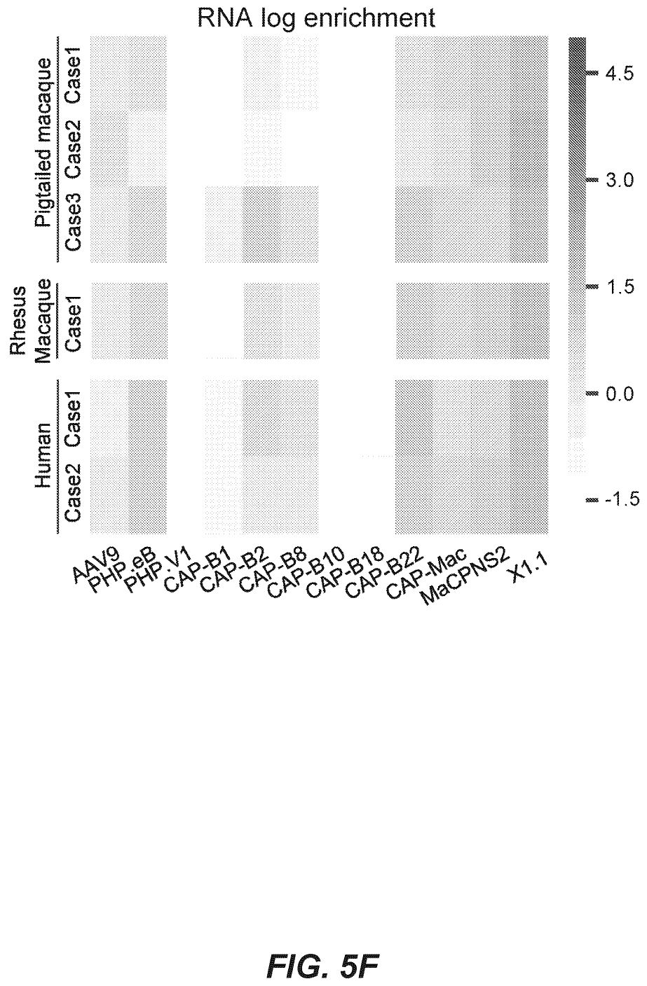

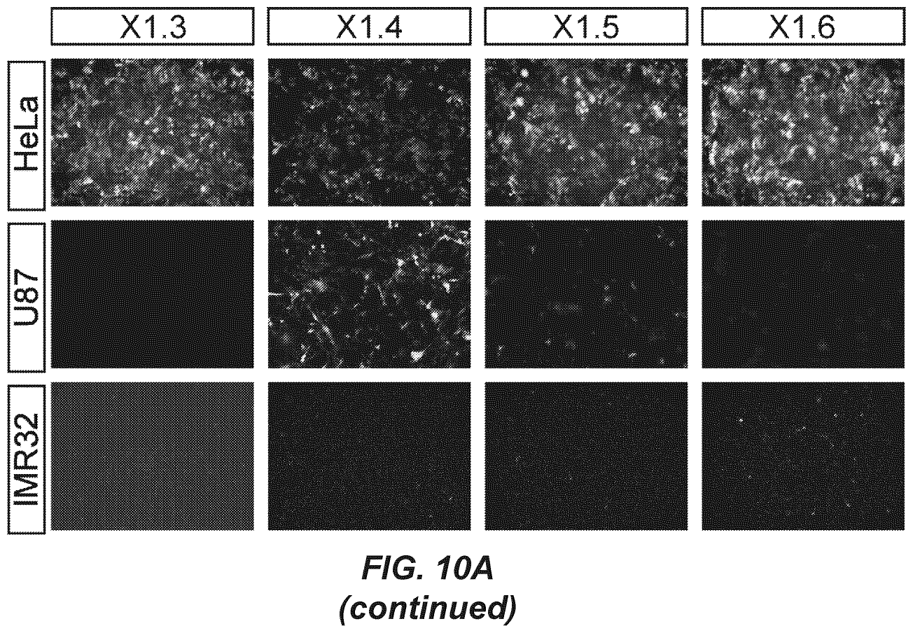

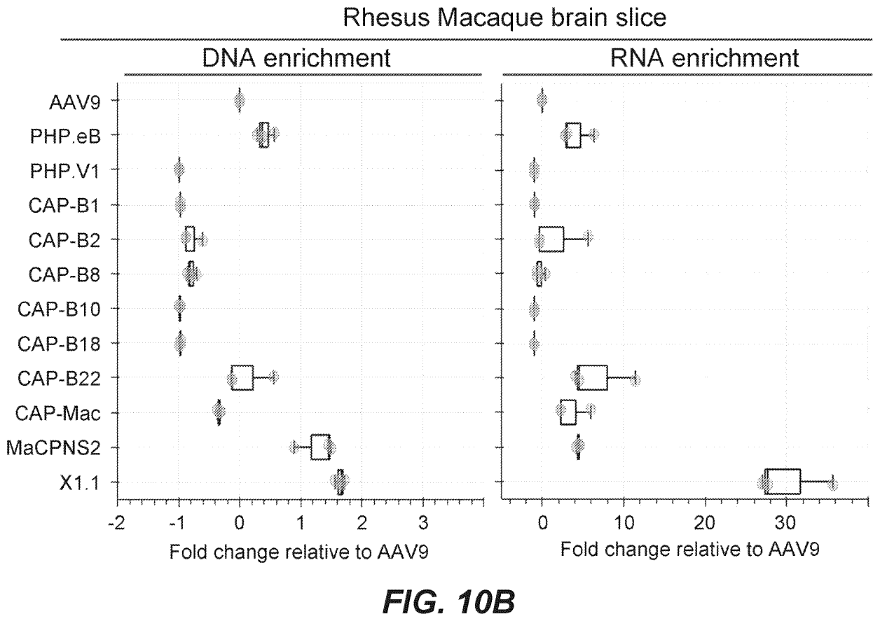

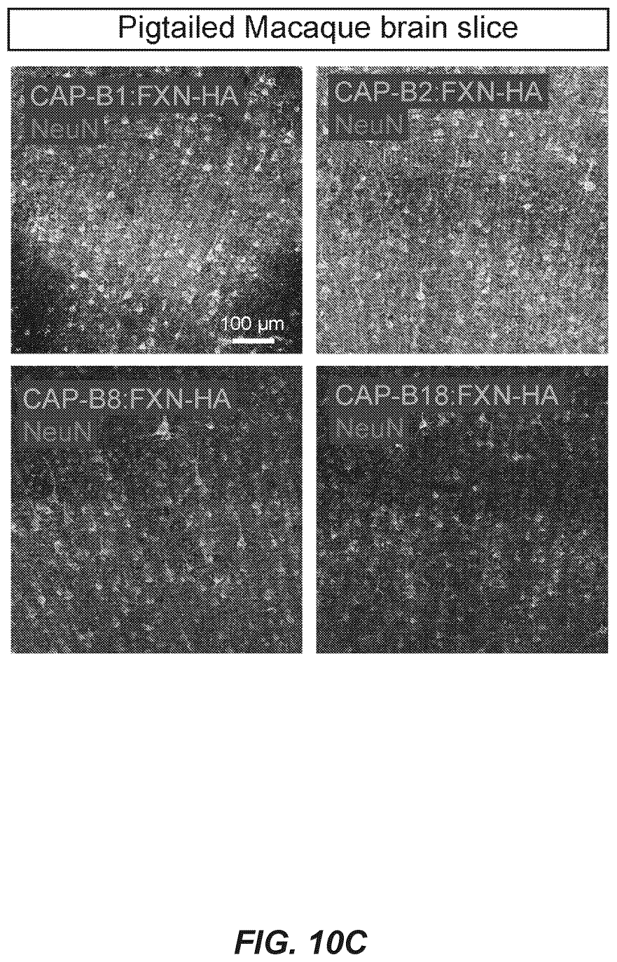

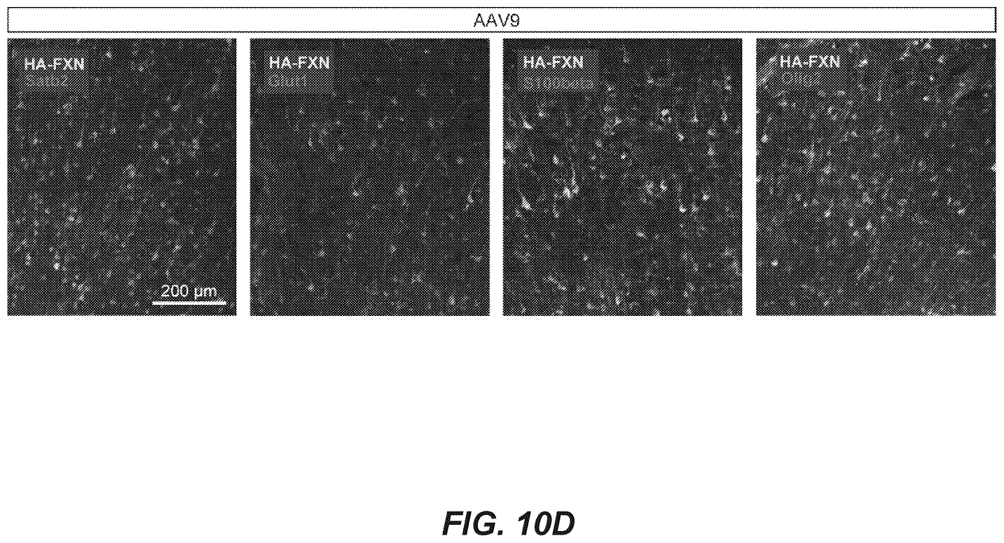

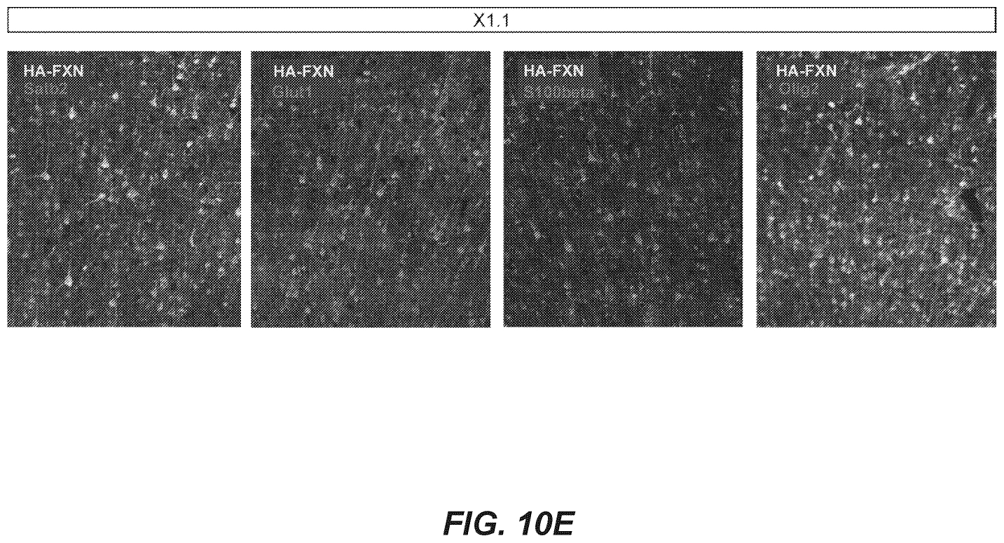

After validating the novel vectors' transduction profiles and demonstrating their functional application in rodent species, translation to higher organisms was studied next. The performance of X1 in transducing isolated human brain microvascular endothelial cells (HBMECs) was measured. At an MOI of 3×10⁴, X1 transduced ˜42% of HBMECs, ˜180 fold higher than its parent AAV9. The further engineered versions X1.1 and X1.2 transduced 44% and 53% of HBMECs, respectively. In contrast, previously-engineered AAV vectors including PHP.eB and BR1 transduced ˜0-2% of HBMECs, showing no obvious improvement in transduction compared to natural serotypes (FIG. 5A). The vectors' performance was next examined at a lower MOI of 3×10³, and an improvement in transduction efficiency for X1.1 and X1.2 was again observed, with ˜22% and ˜28% transduction of HBMECs, respectively (FIG. 5A). In addition to transducing HBMECs, X1 and its engineered derivatives exhibited robust transduction of multiple other human-derived cell lines, including HeLa, U87, C2C12, IMG and IMR-32 (FIG. 10A).

X1.1 Efficiently Transduces Cultured Ex Vivo Brain Slices from Macaque and Human

The strong performance of the novel vectors in HBMECs prompted investigation of their efficacy in a system that may, in some embodiments, better mimic in vivo conditions in non-human primates, e.g., an ex vivo brain slice culture system (FIG. 5B). CAG-Frataxin (FXN)-HA with unique 12 bp barcodes was packaged in a panel of AAVs including AAV9, X1.1, and several other engineered CNS-tropic AAVs. Vectors were injected both tailed macaque (Macaca nemestrina) and cultured in physiological conditions (FIG. 5B). After 7-10 days, DNA and RNA was extracted from the tissue, and the enrichments of the variants from the AAV pool in the tissue using next-generation sequencing (NGS) were calculated. X1.1 showed an ˜3-fold increase and ˜24-fold increase in DNA and RNA, respectively, compared to AAV9. This outperformed all other vectors, including CAP-B10 and CAP-B22, which have been shown to have increased efficiency in transducing the CNS of marmosets after systemic delivery (FIG. 5C). The previously-reported MaCPNS2, which has increased targeting of the macaque CNS, also showed an ˜5-fold increase in RNA compared to AAV9 (FIG. 5C). A higher DNA and RNA presence of X1.1 compared to other AAVs in the pool was also observed in the rhesus macaque brain (FIG. 10B). Immunohistochemistry (IHC) staining of the HA tag confirmed the robust transduction by X1.1 in pig-tailed macaque (FIG. 5D; FIG. 10C). In some embodiments, the cells transduced by X1.1 in the ex vivo brain slices were mostly neuronal (FIG. 5D; FIG. 10D-FIG. 10E). Without being bound by any particular theory, there may be potential differences in tropism across models.

Similar testing was performed on human brain slices originating from a biopsy surgery. X1.1 showed an ˜4-fold and ˜32-fold increase in DNA and RNA, respectively, compared to AAV9, again outperforming several previously-engineered CNS-targeting vectors (FIG. 5E). The strong performance of X1.1 across macaque and human ex vivo slices (FIG. 5F) shows great potential for the capsid in translational applications.

### X1.1 Efficiently Targets the CNS in Rhesus Macaque Following IV Delivery

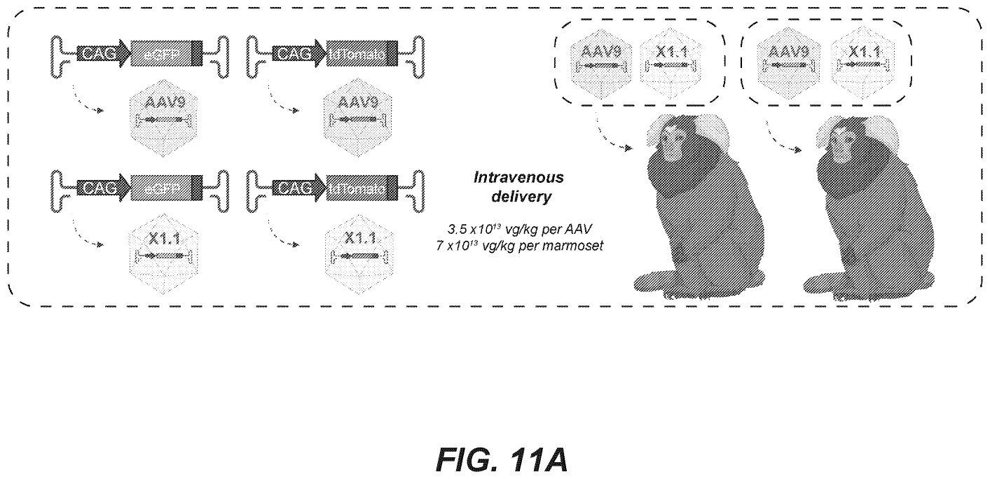

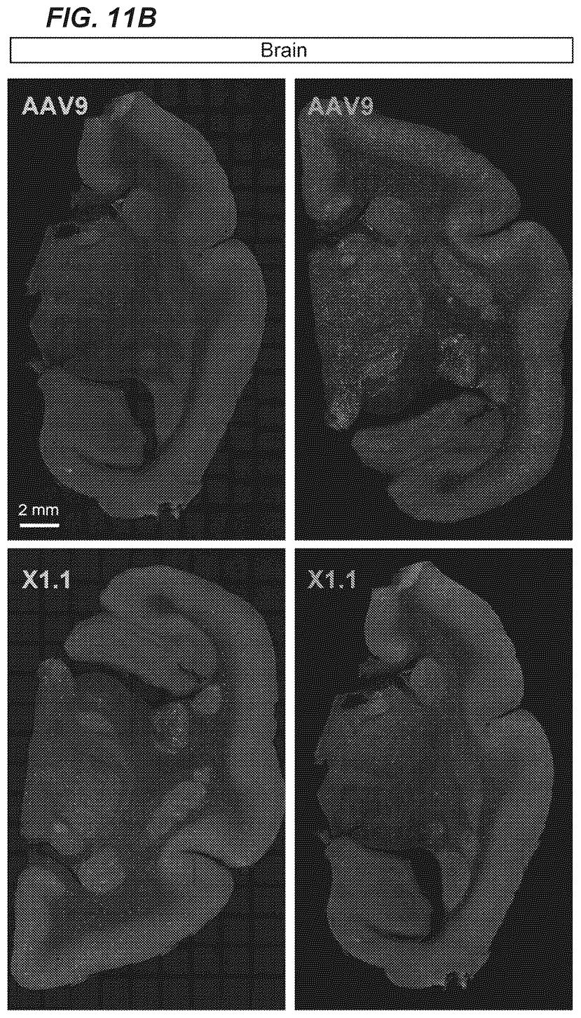

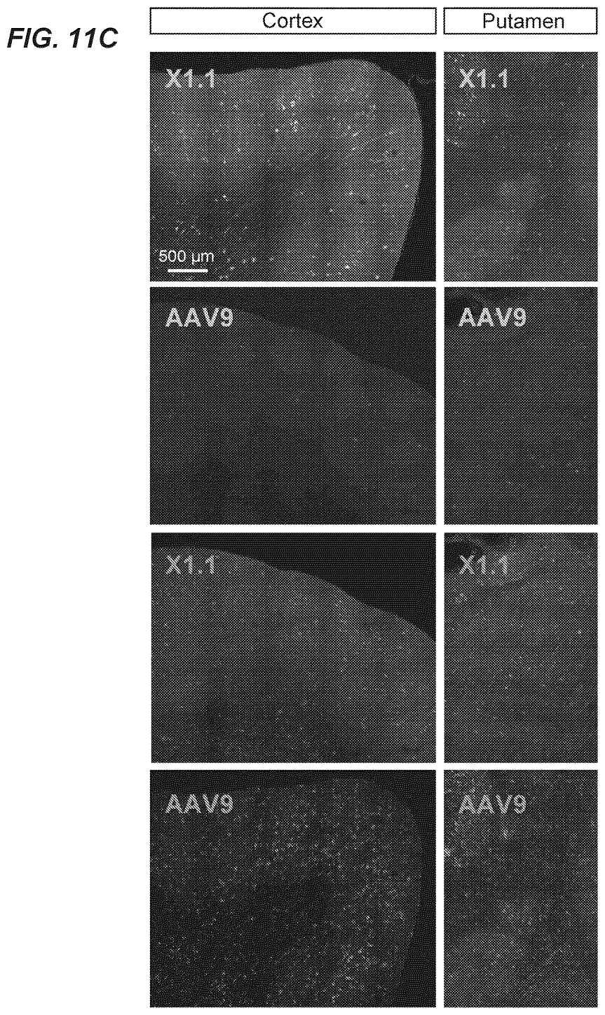

Having validated the new variants' transduction profiles in ex vivo NHP and human brain models, their efficacy in vivo was tested next. They were first tested in marmoset, a New World monkey. To reduce animal subject numbers, two viral capsids (AAV9 and X1.1) packaging different fluorescent reporters (either ssAAV:CAG-eGFP or ssAAV:CAG-tdTomato) in each adult marmoset were tested simultaneously (FIG. 11A). 3 weeks after IV delivery of the viruses, no obvious differences between the CNS transduction of X1.1 and AAV9 were observed (FIG. 11B-FIG. 11C). Without being bound by any particular theory, this result illustrates that, in some embodiments, BBB heterogeneity should be taken into account when performing AAV engineering.

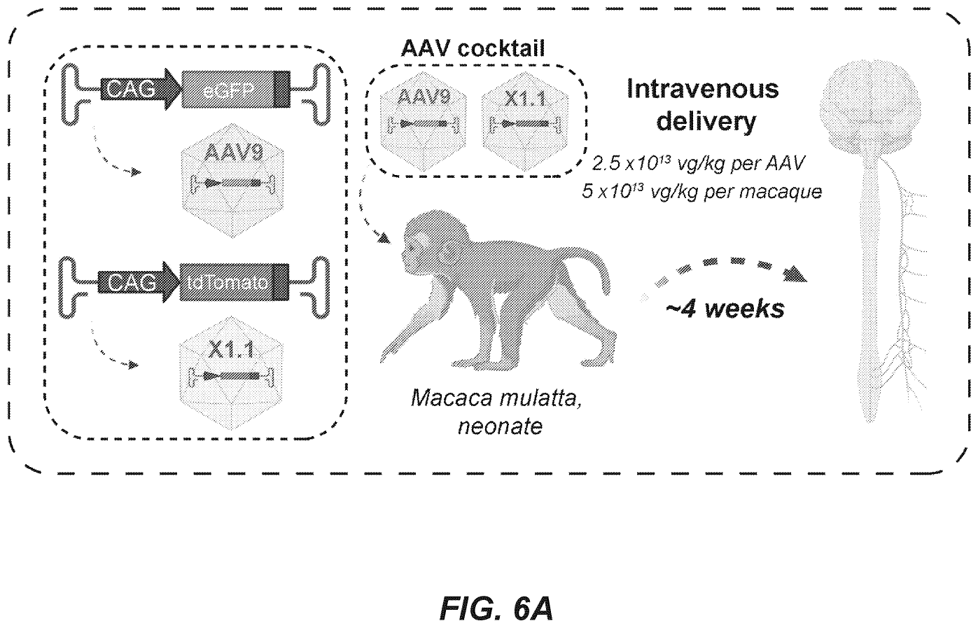

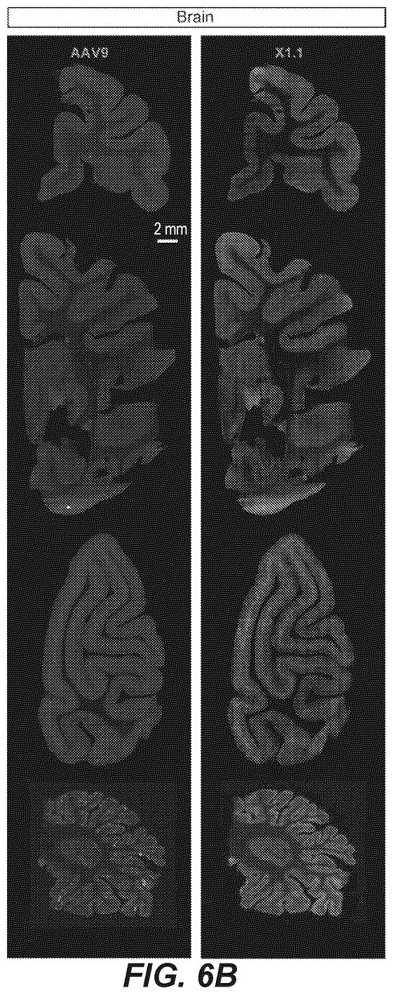

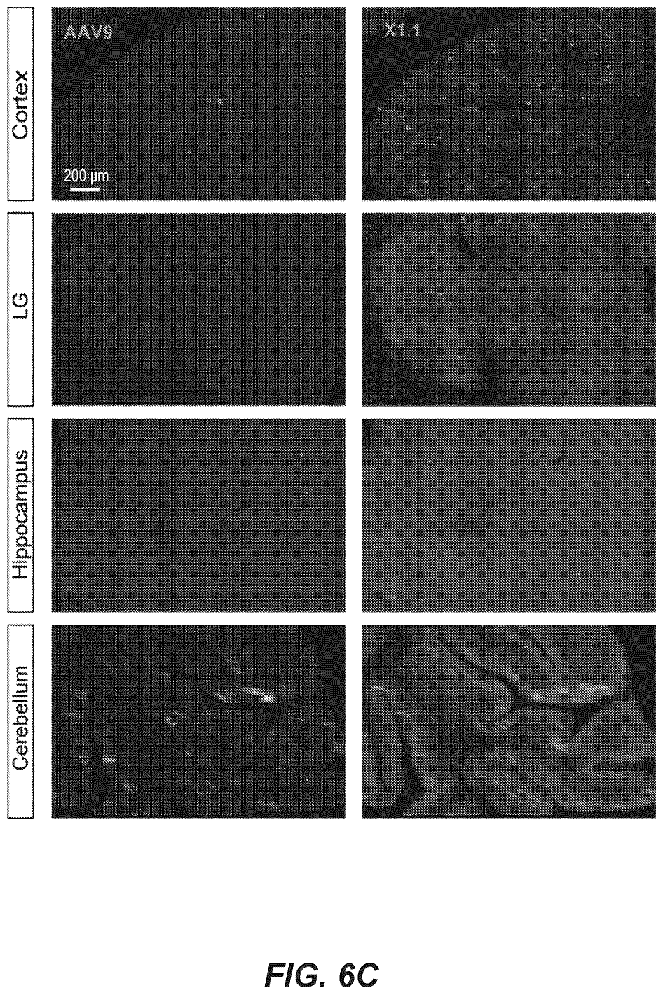

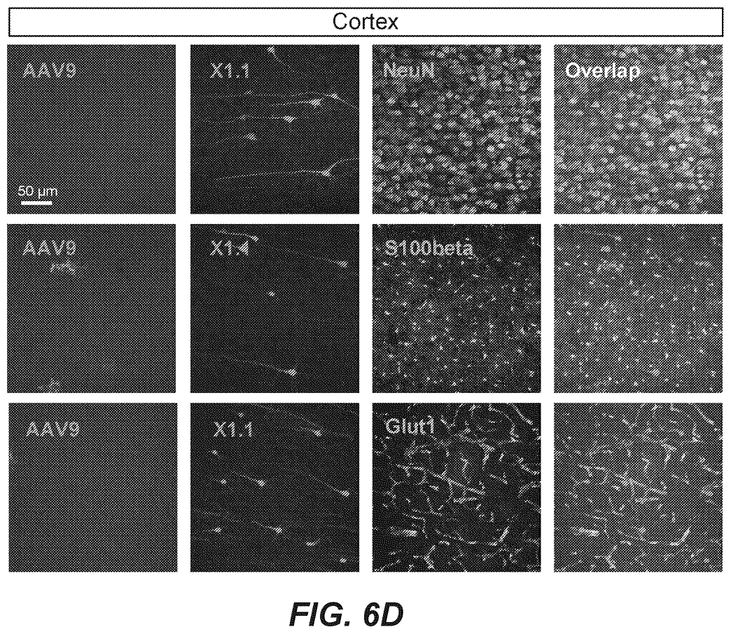

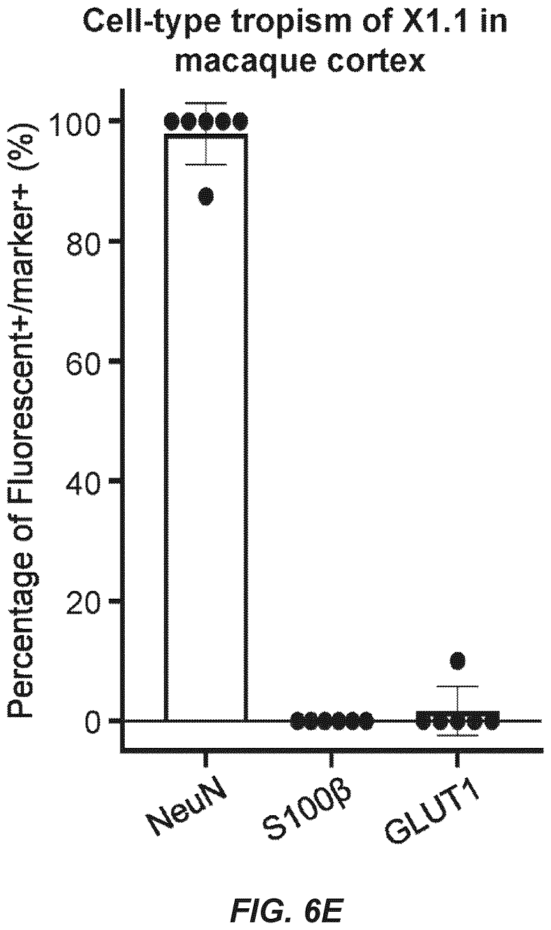

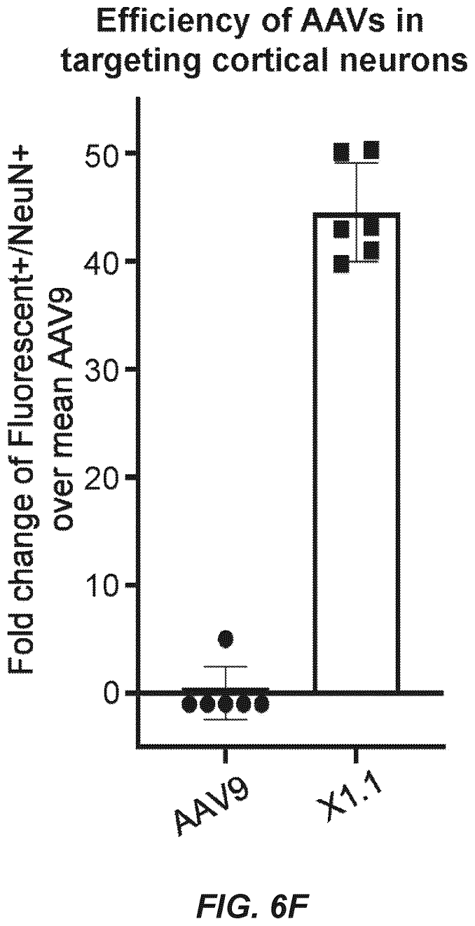

The vectors' efficacy was next tested in the rhesus macaque, an Old World monkey. ssAAV:CAG-eGFP was packaged into AAV9 and ssAAV:CAG-tdTomato into X1.1, then they were intravenously delivered as an AAV cocktail to a neonate (˜1-month-old female) rhesus macaque (female Macaca mulatta, within 10 days of birth, 5×10¹³ vg/kg per macaque, 2.5×10¹³ vg/kg per AAV, n=1 per group) (FIG. 6A). After 4 weeks of expression, robust expression and cerebellum, compared to AAV9 (FIG. 6B-FIG. 6C). Further IHC staining revealed that ˜98% of the cells transduced by X1.1 in the cortex were neurons, while a small proportion of the targeted cells were endothelial or glia cells (FIG. 6D-FIG. 6E). Neuron transduction by X1.1 was ˜45-fold higher than AAV9 (FIG. 6F). The difference between the neurotropic profile of X1.1 in neonate macaque and its endothelial cell-tropic profile in rodents opens up new potential applications. These experiments demonstrate that the new capsid AAV-X1.1 can efficiently transduce the CNS in Old World monkeys, making it a useful vector for translational research of neurological disorders.

Described herein are a family of novel vectors including AAV-X1 and AAV-X1.1, which specifically and efficiently transduced mouse brain endothelial cells with a ubiquitous promoter following systemic administration. This level of specificity in the mouse CNS is unprecedented among both natural and previously-engineered AAV serotypes.

The previously-engineered AAV vector PHP.eB has been widely used for targeting most cell types in mice CNS, while the vector PHP.V1 has been shown to have increased potency for, but not selective targeting of, brain endothelial cells. However, the enhanced CNS tropism of both vectors is absent in a subset of mouse strains, including BALB/cJ. As described herein, similar to PHP.eB, PHP.V1 relies on Ly6a, while AAV-X1 vectors are Ly6a-independent and efficiently target brain endothelial cells across mouse strains. Without being bound by any particular theory, this Ly6a independence shows that these novel vectors may utilize a novel receptor for CNS targeting and adds to the vectors' translational promise, as Ly6a is a murine-specific factor.

A major challenge in achieving successful gene therapy is the presence of neutralizing antibodies against AAVs. The neutralizing antibodies induced by an initial AAV delivery have been reported to persist long after the treatment, which could prevent the successful repeat administration needed for maintaining transgene expression. Serotype switching between administrations can be a potential solution for dealing with neutralizing antibodies against the initially-administered serotype. The 7-mer X1 peptide was successfully transferred from AAV9 to AAV1, yielding AAV1-X1, which transduces brain endothelial cells efficiently following IV delivery. This result shows that the X1 peptide is more modular than the 7-mer peptide of PHP.B. Following administration of AAV1-X1 packaging Ly6a, CBA/J mice were successfully made more permissive for PHP.eB, thus demonstrating the novel vector's ability to introduce a functional receptor and verifying that serotype switching may be a potential solution for sequential AAV administration. The transferability of the AAV9-based X1's 7-mer peptide to AAV1 highlights a general strategy that can be applied to other applications involving repeat administration but also circumvent pre-existing neutralizing antibodies to a certain AAV serotype.

Delivering therapeutic agents, or genetic material encoding therapeutics, across the BBB to treat neurological diseases of the CNS has been a challenging task. Many enzymes, however, are secreted and can exert cross-correction effects. For these, the genetic material can instead be delivered to brain endothelial cells, transforming these cells into a biofactory to produce and distribute therapeutics to other cell types. The novel endothelial cell-tropic vector described herein allows such deliveries in an animal model. The X1.1 vector was used to induce Sparcl1/Hevin protein production in brain endothelial cells and this rescued the thalamocortical synapse loss phenotype of Hevin KO mice. This proof-of-concept supports the brain endothelial cell biofactory model for production of enzymes, antibodies, or other biological therapeutics, providing a novel therapeutic approach for diseases like lysosomal storage disorders.

Previous systemic AAVs for the CNS that were engineered in mice have not always translated to NHPs. Given the X1 vectors' independence of Ly6a, the novel vectors were tested in various NHP species. X1.1 was first tested in marmoset, a New World monkey, and no obvious difference was observed in targeting the CNS compared to AAV9 following systemic delivery. Next, the macaque, an Old World monkey, which is more similar genetically to humans and is widely used in research, including gene therapy was used. The virus was first tested on ex vivo brain slices from the rhesus macaque (Macaca mulatta) and southern pig-tailed macaque (Macaca nemestrina). Greater increases in DNA, RNA, and protein (e.g., FXN) was observed with X1.1 than other CNS vectors including PHP.eB, CAP-B10, and CAP-B22. X1.1 was then intravenously injected into the rhesus macaque and a significant improvement in targeting the CNS compared to AAV9 was observed. Interestingly, the rodent-endothelial-tropic X1.1 efficiently transduces neurons in the macaque brain. The conservation of enhanced CNS tropism across rodents and NHPs is encouraging for X1.1's potential in tackling neurological disorders. X1.1 also has increased efficiency in transducing ex vivo human brain slices compared to other previously-engineered CNS vectors, supporting its use for therapeutic translation.

In some embodiments, e.g., rodents with a healthy BBB, X1 vectors seem to prefer endocytosis to transcytosis at the BBB. Without being bound by any particular theory, this may be due either to their interactions with novel receptors or the vectors' own physiological features. In pericyte-deficient mice where endothelial transcytosis is increased and endothelial cells show occasional hot-spot leakage areas due to altered endothelial cell-cell interaction, increased transduction of astrocytes and neurons was observed. In rhesus macaque, neurons efficiently after crossing the BBB. This distinction in tropism opens up the potential for different applications with X1.1 in endothelial cells and neurons in different species.

In summary, described herein are novel systemic AAV tools to expand understanding of the neurovascular unit across species. The novel vector X1 and its further-engineered family of variants, including X1.1, provide genetic access to brain endothelial cells in mice with unprecedented potency and specificity, and their efficient targeting of the CNS in NHP and human brain slices offers methods for accelerating translational research. The surprising modularity of the X1 variant peptide may allow for application of immunogenically-distinct AAVs at multiple timepoints, and the demonstrated application of the X1 vectors to transform brain endothelial cells into secretory biofactories validates a novel method to deliver therapeutic agents to the CNS.

### Materials and Methods

### Plasmids

### Library Preparation:

Briefly, plasmid rAAV-ΔCap-in-cis-Lox2 (FIG. 1A) was used for building the heptamer insertion (7-mer-i) AAV library. Plasmid pCRII-9Cap-XE was used as a PCR template for the DNA library generation. Plasmid AAV2/9-REP-AAP-ΔCap was used to supplement the AAV library during virus production.

### AAV Capsid Characterization:

The AAV capsid AAV-X1 was built by inserting DNA sequence encoding 7-mer peptides between DNA sequences encoding amino acids at positions 588-589 of AAV9 capsid into the pUCmini-iCAP-PHP.B backbone. The AAV-PHP.V1 capsid gene sequence was described previously. The AAV capsid protein variants AAV-X1.1, AAV-X1.2, and AAV-X1.3 (Table 1) were built by substituting nucleotide sequences encoding amino acids at positions 452-458 of AAV-X1 with sequences encoding the 7-mer peptides shown in Table 2. The AAV capsids AAV-X1.4, AAV-X1.5, and AAV-X1.6 were built by mutation of DNA sequences encoding amino acids at positions 272/386/503 in AAV-X1 to encode Alanine (Table 1). The AAV capsid AAV1-X1 was built by inserting a nucleotide sequence encoding a 7-mer peptide between nucleotide sequence for amino acids 588-589 of the AAV1 cap gene in AAV1-Rep-Cap (Addgene 112862).

For in vivo validation of AAV capsids, the vectors were packaged with a single-stranded (ss) rAAV genome: pAAV:CAG-EGFP, pAAV:CAG-tdTomato (a gift from Edward Boyden, Addgene plasmid #59462). To make pAAV:CAG-EGFP-3xmiR122-TS, 3 copies of the miR122-TS were cut out from plasmid CAG-GCaMP6f-3x-miR204-5p-3x-Fragment (IDT) based off the sequence in the plasmid pAAV:GfaABC1D-Hevin, a gift from Cagla Eroglu's Lab, and subcloned into the plasmid pAAV:CAG-EGFP by replacing the EGFP gene. To make pAAV:CAG-Ly6a, the Ly6a coding sequence from C57BL/6J was synthesized as a gBlocks Gene Fragment (IDT) and subcloned into the plasmid pAAV:CAG-EGFP by replacing the EGFP gene. pAAV-CAG-FXN-HA was chosen for the ex vivo slice study because it contains a ubiquitous CAG promoter and a sequence encoding HA-tagged endogenous human frataxin (FXN) protein and a unique 12 bp barcode sequence. The barcode sequence was used to differentiate different capsid packaging the same construct during the next-generation sequencing (NGS) analysis.

### AAV Capsid Library Generation:

Briefly, the R1 library involved a randomized 21-nucleotide (7×NNK mutagenesis) insertion between nucleotide sequence encoding AAs 588-589 of the AAV9 capsid. The R2 library was built using a synthetic pool method. The R2 library was composed of an equimolar ratio of ˜4000 variants that were recovered from the tissues of interest in R1.

### Animals and Subjects

All animal procedures in mice carried out in this study were approved by the California Institute of Technology Institutional Animal Care and Use Committee (IACUC), Caltech Office of Laboratory Animal Resources (OLAR), Cantonal Veterinary Office Zurich (license number ZH194/2020, 32869/2020), Duke Division of Laboratory Animal Resources (DLAR).

All experimental procedures performed on marmosets were approved by the University of California, San Diego, Institutional Animal Care and Use Committee (IACUC) and in accordance with National Institutes of Health and the American Veterinary Medical Association guidelines. 2 female animals and 1 male animal were used in this study and received intravenous injections of AAVs.

All experimental procedures performed on rhesus macaques were approved by the International Animal Care and Use Committee at the University of California, Davis and the California National Primate Research Center (CNPRC). One infant female animal was used in this study and received intravenous injections of AAVs. All human neurosurgical tissue studies were approved by the Western Institutional Review Board. Human neurosurgical specimens were obtained with informed consent of patients that underwent neocortex resection for the treatment of temporal lobe epilepsy or for tumor removal. Specimens for research were not required for diagnostic purposes and were distal to the pathological focus of the surgical resections.

[symbol]

assigned, and the experimenters were not blinded while performing the experiments unless mentioned otherwise.

### In Vivo Selection and Capsid Library Recovery

For capsid selection in vivo, the virus library was intravenously administered to male and female mice of various Cre transgenic lines (n=2-3 per Cre line) at 3×10¹¹ vg per mouse in R1 selection, and at 3×10¹¹ vg per mouse in R2 selection. Two weeks post injection, mice were euthanized, and the organs of interest were harvested and snap-frozen on dry ice. The tissues were stored at −80° C. To recover capsids from the tissue, the rAAV genome extractions from tissues were processed using Trizol, and the rAAV genomes were recovered by Cre-dependent PCR or Cre-independent PCR. The AAV DNA library, virus library and the libraries recovered from tissue post in vivo selection were processed for NGS.

### Characterization of AAV Vectors

### AAV Vector Production:

The AAV vectors were produced using an optimized vector production protocol and methods known to those of skill in the art. The average yieldₓ was ˜1×10¹² vg per plate. BR1:CAG-GFP was purchased from Signagen (SL116035).

### AAV Vector Administration Across Models and Tissue Harvest:

For the cell-type profiling of the novel AAVs in mice, the AAV vectors were injected intravenously via the retro-orbital route to 6-8 week old adult mice at a dose of 0.1-1×10¹² vg per mouse. The retro-orbital injections were performed as described previously. The expression times were ˜3 weeks from the time of injection. The dosage and expression time were kept consistent across different experimental groups unless noted otherwise. To harvest the tissues of interest, the mice were anesthetized with Euthasol (pentobarbital sodium and phenytoin sodium solution, Virbac AH) and transcardially perfused using 30-50 mL of 0.1 M phosphate buffered saline (PBS) (pH 7.4), followed by 30-50 mL of 4% paraformaldehyde (PFA) in 0.1 M PBS. The organs were collected and post-fixed 24-48 h in 4% PFA at 4° C. Following this, the tissues were washed with 0.1 M PBS twice and stored in fresh PBS-azide (0.1 M PBS containing 0.05% sodium azide) at 4° C.

In the Hevin experiment, 4-month-old Hevin KO mice were retro-orbitally injected with either AAV-X1.1:CAG-Hevin-HA or AAV-X1.1:CAG-eGFP (1×10¹² vg per mouse). After 3 weeks, the mice were perfused and brains were extracted for synapse assay.

In the experiment with pericyte-deficient mice, 4-5 month-old PDGFB-retention motif knock out mice (Pdgfb^{ret/ret}) in a C57BL6/J genetic background were used. 3×10¹¹ vg per mice for X1 and 1E12 vg per mice for X1.1 were injected into mice via the tail followed by 5 min perfusion with 4% PFA in PBS, pH 7.2. Brains were collected and post-fixed in 4% PFA in PBS, pH 7.2 at 4° C. for 6 h.

Female rats were used (150-200 g) for experiments. 1×10¹³ vg of the virus was delivered intravenously through the tail vein under light anesthesia. The injected volume was 0.5 ml containing the required number of particles. After 21 days the animals were perfused using 4% PFA solution and PBS. Brains were collected.

Marmoset monkeys were anesthetized using an intramuscular Ketamine (20 mg/kg) and Acepromazine (0.5 mg/kg) injection. An intravenous catheter was placed in the saphenous vein of the hind leg and flushed with ˜2 mL of LRS (Lactated Ringer's solution) for 2 min. Viruses were pooled together in a single syringe (˜500-900 μL) and infused at a rate of 200 μL/min into the catheter. Following the infusion, the catheter was flushed with ˜3 mL of LRS for 2 min and removed. The animal was then returned to a recovery cage. Following an incubation period of 4-6 weeks post viral injection, the animals were euthanized by injecting pentobarbital intraperitoneally. Two researchers worked in parallel to harvest the tissue to limit degradation as quickly as possible. Each organ—brain, lungs, kidneys, etc.—was removed and separated into two parts. One half of the tissue was flash-frozen in 2-methylbutane that was chilled with dry ice to preserve mRNA and DNA in the harvested tissues. The other half of the tissue was fixed in 4% PFA solution for estimation of protein expression. Flash-frozen tissue samples were transferred to a −80° C. freezer, while PFA-fixed tissue samples were stored in a 4° C. fridge.

One female rhesus macaque was injected within 10 days of birth. Prior to injection, the animal was anesthetized with ketamine (0.1 mL) and the skin over the saphenous vein was shaved and sanitized. AAVs (2.5×10¹³ vg/kg) were slowly infused into the saphenous vein for ˜1 min in <0.75 mL of 0.1 M PBS. The animal was monitored while they recovered from anesthesia in their home enclosure, and daily for the remainder of the study. The monkey was individually housed within sight and sound of conspecifics.

Tissues were collected 4 weeks post AAV administration. The animal was deeply anesthetized and euthanized using sodium pentobarbital in accordance with guidelines for humane euthanasia of animals at the CNPRC. The whole body was perfused with ice cold RNase-free 0.1 M PBS. The brain was removed from the skull and blocked into 4 mm thick slabs in the coronal plane. Brain slabs and organs were subsequently post-fixed in 4% PFA for 48 h. One hemisphere of the animal was cryoprotected in 10%, 15%, and 30% sucrose in 0.1 M PBS.

All procedures involving non-human primates conformed to the guidelines provided by the US National Institutes of Health. Ex vivo brain slice culture experiments were performed on temporal cortex tissue from adult Macaca nemestrina or Macaca mulatta animals housed at the Washington National Primate Research Center. These brain samples were obtained through the Tissue Distribution Program operating under approved University of Washington IACUC protocol number 4277-01. All human neurosurgical tissue studies were approved by the Western Institutional Review Board. Human neurosurgical specimens were obtained with informed consent of patients that underwent neocortex resection for the treatment of temporal lobe epilepsy or for tumor removal. Specimens for research were not required for diagnostic purposes and were distal to the pathological focus of the surgical resections.

Human and macaque brain slices were prepared using the NMDG protective recovery method. Human neurosurgical tissue or macaque brain tissue specimens were placed in carbogenated NMDG artificial cerebral spinal (ACSF) solution containing (in mM): 92 NMDG, 2.5 KCl, 1.25 NaH₂PO₄, 30 NaHCO₃, 20 HEPES, 25 glucose, 2 thiourea, 5 Na-ascorbate, 3 Na-pyruvate, 0.5 CaCl₂·4H₂O and 10 MgSO₄·7H₂O. Brain slices were prepared on a VF-200 Compresstome at 300 m thickness using a zirconium ceramic blade (EF-INZ10, Cadence) and then underwent warmed recovery in carbogenated NMDG aCSF at 32-34° C. for 12 minutes. Human and macaque brain slices were placed on membrane inserts in 6 well sterile culture plates, and wells were filled with slice culture medium consisting of 8.4 g/L MEM Eagle medium, 20% heat-inactivated horse serum, 30 mM HEPES, 13 mM D-glucose, 15 mM NaHCO₃, 1 mM ascorbic acid, 2 mM MgSO₄·7H2O, 1 mM CaCl₂·4H₂O, 0.5 mM GlutaMAX-I, and 1 mg/L insulin. The slice culture medium was carefully adjusted to pH 7.2-7.3, osmolality of 300-310 mOsmoles/Kg by addition of pure H₂O and then sterile-filtered. Culture plates were placed in a humidified 5% CO₂ incubator at 35° C. and the slice culture medium was replaced every 2-3 days until end point analysis. 1-3 hours after plating, brain slices were infected by direct application of ˜2.5×10¹⁰ vg of concentrated AAV viral particles distributed over the slice surface.

### Immunohistochemistry and Imaging

### Immunohistochemistry:

In the AAV characterization experiment in mice, tissue sections, typically 100-μm thick, were first incubated in blocking buffer (10% normal donkey serum (NDS), 0.1% Triton X-100, and 0.01% sodium azide in 0.1 M PBS, pH 7.4) with primary antibodies at appropriate dilutions for 24 h at room temperature (RT) on a rocker. After primary antibody incubation, the tissues were washed 1-3 times with wash buffer 1 (0.1% Triton X-100 in 0.1 M blocking buffer with the secondary antibodies at appropriate dilutions for 12-24h at RT and then washed 3 times in 0.1 M PBS over a total duration of 5-6h. When performing DNA staining, the tissues were incubated with 4′, 6-diamidino-2-phenylindole (DAPI) (Sigma Aldrich, 10236276001, 1:1,000) in 0.1 M PBS for 15 min followed by a single wash for 10 min in 0.1 M PBS. The DAPI and/or antibody-stained tissue sections were mounted with ProLong Diamond Antifade Mountant (ThermoFisher Scientific, P36970) before imaging them under the microscope. The images were acquired with a Zeiss LSM 880 confocal microscope using the following objectives: Plan-Apochromat 10×0.45 M27 (working distance 2.0 mm), and Plan-Apochromat 25×0.8 Imm Corr DIC M27 multi-immersion. The liver images were acquired with a Keyence BZ-X700 microscope using a 10× objective. The images were then processed in the following image processing software: Zen Black 2.3 SP1 (for Zeiss confocal images) and BZ-X Analyzer (for Keyence images).

In the rat experiment, whole brains were imaged using serial section two-photon microscopy. The microscope was controlled by ScanImage Basic (Vidrio Technologies, USA) using BakingTray, a custom software wrapper for setting up the imaging parameters. 50-60 μm slices were cut and 7-9 optical planes were imaged using a 16× objective. Images were assembled using StitchIt.

In the Hevin experiment, for synaptic puncta analysis of mouse primary visual cortex (area V1), brains were cryosectioned at 25 μm using Leica CM3050S (Leica, Germany). Tissue sections were washed and permeabilized in TBS with 0.2% Triton-X 100 three times at room temperature followed by blocking in 5% Normal Goat Serum (NGS) for 1 hr at room temperature. To label pre- and post-synaptic proteins, VGluT2 (Synaptic Systems; Cat #135 404) and PSD95 (Thermo Fisher; Cat #51-6900) antibodies were used, respectively. Primary antibodies were diluted in 5% NGS containing TBST and incubated overnight at 4° C. Secondary antibodies (Alexa Fluor conjugated; Invitrogen) were added in TBST with 5% NGS for 2 hr at room temperature. Slides were mounted in Vectashield with DAPI (Vector Laboratories, CA). Images were acquired with Olympus FV 3000 inverted confocal microscope using high magnification 60× objective plus 1.64× optical zoom z-stack images containing 15 optical sections spaced 0.33 μm apart. During the post processing of captured images, each z-stack was converted into 5 maximum projection images (MPI) by combining three optical sections using ImageJ software. The number of co-localized excitatory thalamocortical (VGluT2/PSD95) synaptic puncta were obtained using the ImageJ plugin Puncta Analyzer.

In the pericyte-deficient mice experiment, coronal vibratome sections (60 m) of brains were cut using the Leica VT1000S. Free floating brain sections were incubated in the overnight at 4° C., followed by incubation in primary antibody solution for two nights at 4° C., and subsequently in secondary antibody solution, overnight at 4° C. Sections were incubated with DAPI (4′,6-Diamidino-2-phenylindole dihydrochloride) solution (D9542, Sigma-Aldrich, diluted 1:10000) for 7 minutes at RT and subsequently mounted in ProLong Gold Antifade mounting medium (cat. #P36930), Life Technologies). Images were taken with Leica SP8 inverse, 20× objective PL APO CS (NA 0.7) (Leica Microsystems). Wide-field images were generated using Slidescanner Zeiss Axio Scan.Z1 (Leica Microsystems). Image processing was done using Fuji and Zen2. All confocal images are represented as maximum intensity projections.

In the marmoset and macaque experiment, coronal sections (100 m) of brains were cut using the Leica VT1000S. Sections (50-100 m) of gut, DRG and spinal cord were cut using a cryostat (Leica Biosystems). Tissues were stained with relevant antibodies following a similar protocol as in the mouse characterization experiment. The images were acquired with a Zeiss LSM 880 confocal microscope using the following objectives: Plan-Apochromat 10×0.45 M27 (working distance 2.0 mm), and Plan-Apochromat 25×0.8 Imm Corr DIC M27 multi-immersion. The images were then processed in the Zen Black 2.3 SP1 (for Zeiss confocal images).

In the primate ex vivo slice culture experiments, following 7-10 days post virus infection, temporal cortex brain slices were fixed in 4% PFA for 24 hours at 4° C. After PFA fixation, slices were transferred into 30% sucrose in water for >24 hours and then sub-sectioned on a sliding microtome to 15-30 μm for immunostaining using the following antibodies: mouse anti-HA (Biolegend catalog #901513, 1:1000) and rabbit anti-NeuN (Millipore catalog #ABN78, 1:2000). In a subset of IHC experiments co-immunostaning was performed with additional cell type marker antibodies including the following: rabbit anti-Glut1 (Millipore catalog #07-1401, 1:1000), rabbit anti-Olig2 (Abcam catalog #AB9610, 1:1000), and mouse anti-S100B (Millipore catalog #S2532, 1:1000). Rat anti-HA (Roche catalog #3F10, 1:1000) was used instead of mouse anti-HA to circumvent antibody cross reactivity issues observed with Olig2 and other cell type markers. Secondary antibodies included Goat anti-mouse, Goat anti-rabbit, or Goat anti-rat Alexa Fluor conjugated antibodies (A488, A555, A647 from ThermoFisher) as needed. Slices were incubated in 1 μg/mL DAPI solution and mounted on 1×3 inch slides with Prolong Gold mounting medium. Region of interest (ROI) in the neocortical grey matter spanning L3/4/5 were imaged on an Olympus FV3000 confocal microscope using 405 nm, 561 nm, and 640 nm laser lines. Z-stack images were acquired at 1 μm step sized through the slice thickness and collapsed to made maximum intensity projection expression patterns and imaged at matched settings to directly compare across the capsid variants.

### Protein Production

Ly6a-Fc was produced in Expi293F suspension cells grown in Expi293 Expression Medium (Thermo Fisher Scientific) in a 37° C., 5% CO₂ incubator with 130 rpm shaking. Transfection was performed with Expifectamine according to manufacturer's instructions (Thermo Fisher Scientific). Following harvesting of cell conditioned media, 1 M Tris, pH 8.0 was added to a final concentration of 20 mM. Ni-NTA Agarose (QIAGEN) was added to ˜5% conditioned media volume. 1 M sterile PBS, pH 7.2 (GIBCO) was added to ˜3× conditioned media volume. The mixture was stirred overnight at 4° C. Ni-NTA agarose beads were collected in a Buchner funnel and washed with ˜300 mL protein wash buffer (30 mM HEPES, pH 7.2, 150 mM NaCl, 20 mM imidazole). Beads were transferred to an Econo-Pak Chromatography column (Bio-Rad) and protein was eluted in 15 mL of elution buffer (30 mM HEPES, pH 7.2, 150 mM NaCl, 200 mM imidazole). Proteins were concentrated using Amicon Ultracel 10K filters (Millipore) and absorbance at 280 nm was measured using a Nanodrop 2000 spectrophotometer (Thermo Fisher Scientific) to determine protein concentration.

### Surface Plasmon Resonance (SPR)

Experiments were performed using a Sierra SPR-32 Pro (Bruker). Ly6a-Fc fusion protein in HBS-P+ buffer (GE Healthcare) was immobilized to a protein A sensor chip at a capture level of approximately 1200-1500 response units (RUs). Two-fold dilutions of rAAV beginning at 4×10¹²v.g. mL⁻¹ were injected at a flow rate of 10 μl min⁻¹ with a contact time of 240 s and a dissociation time of 600 s. The protein A sensor chip was regenerated with 10 mM glycine pH 1.5 after each cycle. Kinetic data were double reference subtracted.

### Bulk Sequencing for Capsid Enrichment in Ex Vivo Tissue

Ex vivo NHP or human brain slice cultures were either infected with the pool of viruses with equivalent molar ratio or infected with PHP.eB packaging non-CAG-FXN-HA construct. After 7 or 10 days in vitro, the tissues were snap frozen and shipped to Caltech on dry ice. Tissues were immediately stored at −80° C. upon receipt. RNA was extracted from the whole tissue or half of the tissue following a slightly modified Phenol/Chloroform protocol. RNA was further cleaned by treating with DNase using RNA Clean & Concentrator-5 kit (Zymo), and reverse-transcription was done on up to 1 μg purified RNA using Superscript IV VILO Master Mix (ThermoFisher). If DNA was needed from the tissue, DNA was extracted from the other half of the tissue using QIAamp DNA Mini Kit (Qiagen) followed the manufacturer's instructions.

[symbol]

DNA using primers of 5′-TGGACCTAAGCGTTATGACTGGAC-3′ (SEQ ID NO: 25) and 5′-GGAGCAACATAGTTAAGAATACCAGTCAATC-3′ (SEQ ID NO: 26) and PCR was performed using Q5 2× Master Mix (New England BioLabs) at 25 cycles of 98° C. for 10s, 63° C. for 15s, and 72° C. for 20s. Each sample was run in up to 5 reactions using up to 50 ng of cDNA or DNA, each, as a template. After PCR, samples were purified using DNA Clean & Concentrator-25 kit (Zymo). The barcoded F×N region was further recovered and the adaptor sequence was added by performing PCR using primers of 5′-ACGCTCTTCCGATCTTGTTCCAGATTACGCTTGAG-3′ (SEQ ID NO: 27) and 5′-TGTGCTCTTCCGATCTTGTAATCCAGAGGTTGATTATCG-3′ (SEQ ID NO: 28) at 10 cycles of 98° C. for 10s, 55° C. for 15s, and 72° C. for 20s. Samples were then purified using DNA Clean & Concentrator-25 kit. Index sets in the NEBNext Dual Index Primers (New England BioLabs) were carefully chosen and added to the barcoded F×N region by performing PCR at 10 cycles of 98° C. for 10s, 60° C. for 15s, and 72° C. for 20s. To further separate the sequence for later next-generation sequencing (NGS), the PCR samples were run on a 2% low-melting-point agarose gel for separation and recovery of the 210 bp band.

NGS was performed on an Illumina MiSeq Next Generation Sequencer (Illumina) using a 150-cycle MiSeq Reagent Kit v3 (Illumina) following the manufacturer's procedure. All samples were pooled in equal ratio to a 4 nM library. 10% 20 pM PhiX control was spiked in to add diversity to the library. Demultiplexing was done by BaseSpace Sequence Hub and the barcode counting analysis was performed using in-house Python code. For each brain slice culture, the enrichment of capsid variants was calculated by the ratio of the counts of their corresponding barcode to the counts of the corresponding barcode to the internal control capsid (e.g., AAV9 or PHP.eB). To correct any potential error due to titer determination, PCR amplification, or sequencing, DNA from the same pool of virus that was used for the brain slice culture infection was extracted, amplified, and included in the MiSeq NGS and analysis. The enrichment of the capsid variants was then normalized by the input viral DNA in the pool.

### Data Analysis

Quantification of AAV transduction in vivo: The quantification of AAV transduction across tissues was carried out by manually counting fluorescent expression resulting from the AAV genome. ImageJ was used for this purpose.

NGS data alignment, processing and analysis: The raw fastq DNA files were aligned to AAV9 capsid template using a custom alignment software. The NGS data analysis was carried out using a custom data-processing pipeline with scripts written in Python and using plotting software such as Plotly, Seaborn, and GraphPad PRISM 7.05. The AAV9 capsid using the following formula: Enrichment score of variant “x”=log 10 [(Variant “Cx” RC in tissue library1/Sum of variants N RC in library1)/(Variant 1 RC in virus library/Sum of variants N RC in virus library)]. Where N is the total number of variants in a library. The fold-change of a variant “x” to AAV9=(The enrichment of “x”−The enrichment of AAV9)/|The enrichment of AAV9|.

TABLE 1
Amino Acid Sequence of AAV capsids
and capsid variants

| | SEQ | |
|---|---|---|
| | ID | |
| Name | NO | Sequence |
| AAV9 | 1 | MAADGYLPDWLEDNLSEGIREWWALKPGAPQPKA |
| | | NQQHQDNARGLVLPGYKYLGPGNGLDKGEPVNAAD |
| | | AAALEHDKAYDQQLKAGDNPYLKYNHADAEFQERL |
| | | KEDTSFGGNLGRAVFQAKKRLLEPLGLVEEAAKTA |
| | | PGKKRPVEQSPQEPDSSAGIGKSGAQPAKKRLNFG |
| | | QTGDTESVPDPQPIGEPPAAPSGVGSLTMASGGGA |
| | | PVADNNEGADGVGSSSGNWHCDSQWLGDRVITTST |
| | | RTWALPTYNNHLYKQISNSTSGGSSNDNAYFGYST |
| | | PWGYFDFNRFHCHFSPRDWQRLINNNWGFRPKRLN |
| | | FKLFNIQVKEVTDNNGVKTIANNLTSTVQVFTDSD |
| | | YQLPYVLGSAHEGCLPPFPADVFMIPQYGYLTLND |
| | | GSQAVGRSSFYCLEYFPSQMLRTGNNFQFSYEFEN |
| | | VPFHSSYAHSQSLDRLMNPLIDQYLYYLSRTINGS |
| | | GQNQQTLKFSVAGPSNMAVQGRNYIPGPSYRQQRV |
| | | STTVTQNNNSEFAWPGASSWALNGRNSLMNPGPAM |
| | | ASHKEGEDRFFPLSGSLIFGKQGTGRDNVDADKVM |
| | | ITNEEEIKTTNPVATESYGQVATNHQSAQAQAQTG |
| | | WVQNQGILPGMVWQDRDVYLQGPIWAKIPHTDGNF |
| | | HPSPLMGGFGMKHPPPQILIKNTPVPADPPTAFNK |
| | | DKLNSFITQYSTGQVSVEIEWELQKENSKRWNPEI |
| | | QYTSNYYKSNNVEFAVNTEGVYSEPRPIGTRYLTR |
| | | NL |
| AAV1 | 2 | MAADGYLPDWLEDNLSEGIREWWDLKPGAPKPKAN |
| | | QQKQDDGRGLVLPGYKYLGPFNGLDKGEPVNAADA |
| | | AALEHDKAYDQQLKAGDNPYLRYNHADAEFQERLQ |
| | | EDTSFGGNLGRAVFQAKKRVLEPLGLVEEGAKTAP |
| | | GKKRPVEQSPQEPDSSSGIGKTGQQPAKKRLNFGQ |
| | | TGDSESVPDPQPLGEPPATPAAVGPTTMASGGGAP |
| | | MADNNEGADGVGNASGNWHCDSTWLGDRVITTSTR |
| | | TWALPTYNNHLYKQISSASTGASNDNHYFGYSTPW |
| | | GYFDFNRFHCHFSPRDWQRLINNNWGFRPKRLNFK |
| | | LFNIQVKEVTTNDGVTTIANNLTSTVQVFSDSEYQ |
| | | LPYVLGSAHQGCLPPFPADVFMIPQYGYLTLNNGS |
| | | QAVGRSSFYCLEYFPSQMLRTGNNFTFSYTFEEVP |
| | | FHSSYAHSQSLDRLMNPLIDQYLYYLNRTQNQSGS |
| | | AQNKDLLFSRGSPAGMSVQPKNWLPGPCYRQQRVS |
| | | KTKTDNNNSNFTWTGASKYNLNGRESIINPGTAMA |
| | | SHKDDEDKFFPMSGVMIFGKESAGASNTALDNVMI |
| | | TDEEEIKATNPVATERFGTVAVNFQSSSTDPATGD |
| | | VHAMGALPGMVWQDRDVYLQGPIWAKIPHTDGHFH |
| | | PSPLMGGFGLKNPPPQILIKNTPVPANPPAEFSAT |
| | | KFASFITQYSTGQVSVEIEWELQKENSKRWNPEVQ |
| | | YTSNYAKSANVDFTVDNNGLYTEPRPIGTRYLTRP |
| | | L |
| X1 | 3 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSYIPGPSYRQQRVSTTVTQNNNSEFAW |
| | | PGASSWALNGRNSLMNPGPAMASHKEGEDRFFPLS |
| | | GSLIFGKQGTGRDNVDADKVMITNEEEIKTTNPVA |
| | | TESYGQVATNHQSAQGNNTRSVAQAQTGWVQNQGI |
| | | LPGMVWQDRDVYLQGPIWAKIPHTDGNFHPSPLMG |
| | | GFGMKHPPPQILIKNTPVPADPPTAFNKDKLNSFI |
| | | TQYSTGQVSVEIEWELQKENSKRWNPEIQYTSNYY |
| | | KSNNVEFAVNTEGVYSEPRPIGTRYLTRNL |
| X1.1 | 4 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTIDGAA |
| | | TKNQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRSV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |
| X1.2 | 5 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTIDGQS |
| | | SKSQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRSV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |
| X1.3 | 6 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTILQTS |
| | | SPGQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRSV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNN |
| X1.4 | 7 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTA |
| | | PGKKRPVEQSPQEPDSSAGIGKSGAQPAKKRLNFG |
| | | QTGDTESVPDPQPIGEPPAAPSGVGSLTMASGGGA |
| | | PVADNNEGADGVGSSSGNWHCDSQWLGDRVITTST |
| | | RTWALPTYNNHLYKQISNSTSGGSSNDAAYFGYST |
| | | PWGYFDFNRFHCHFSPRDWQRLINNNWGFRPKRLN |
| | | FKLFNIQVKEVTDNNGVKTIANNLTSTVQVFTDSD |
| | | YQLPYVLGSAHEGCLPPFPADVFMIPQYGYLTLND |
| | | GSQAVGRSSFYCLEYFPSQMLRTGNNFQFSYEFEN |
| | | VPFHSSYAHSQSLDRLMNPLIDQYLYYLSRTINGS |
| | | GQNQQTLKFSVAGPSNMAVQGRNYIPGPSYRQQRV |
| | | STTVTQNNNSEFAWPGASSWALNGRNSLMNPGPAM |
| | | ASHKEGEDRFFPLSGSLIFGKQGTGRDNVDADKVM |
| | | ITNEEEIKTTNPVATESYGQVATNHQSAQGNNTRS |
| | | VAQAQTGWVQNQGILPGMVWQDRDVYLQGPIWAKI |
| | | PHTDGNFHPSPLMGGFGMKHPPPQILIKNTPVPAD |
| | | PPTAFNKDKLNSFITQYSTGQVSVEIEWELQKENS |
| | | KRWNPEIQYTSNYYKSNNVEFAVNTEGVYSEPRPI |
| | | GTRYLTRNL |
| X1.5 | 8 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTINGSG |
| | | QNQQTLKFSVAGPSNMAVQGRNYIPGPSYRQQRVS |
| | | TTVTQNNNSEFAAPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRSV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |
| X1.6 | 9 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | AQAVGRSSFYCLEYFPSQMLRTGNNFQFSYEFENV |
| | | PFHSSYAHSQSLDRLMNPLIDQYLYYLSRTINGSG |
| | | QNQQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRSV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |
| AAV- | 10 | MAADGYLPDWLEDNLSEGIREWWDLKPGAPKPKAN |
| X1 | | QQKQDDGRGLVLPGYKYLGPFNGLDKGEPVNAADA |
| | | AALEHDKAYDQQLKAGDNPYLRYNHADAEFQERLQ |
| | | EDTSFGGNLGRAVFQAKKRVLEPLGLVEEGAKTAP |
| | | GKKRPVEQSPQEPDSSSGIGKTGQQPAKKRLNFGQ |
| | | TGDSESVPDPQPLGEPPATPAAVGPTTMASGGGAP |
| | | MADNNEGADGVGNASGNWHCDSTWFDFNRFHCHFS |
| | | PRDWQRLINNNWGFRPKRLNFKLFNIQVKEVTTND |
| | | GVTTIANNLTSTVQVFSDSEYQLPYVLGSAHQGCL |
| | | PPFPADVFMIPQYGYLTLNNGSQAVGRSSFYCLEY |
| | | FPSQMLRTGNNFTFSYTFEEVPFHSSYAHSQSLDR |
| | | LMNPLIDQYLYYLNRTQNQSGSAQNKDLLFSRGSP |
| | | AGMSVQPKNWLPGPCYRQQRVSKTKTDNNNSNFTW |
| | | TGASKYNLNGRESIINPGTAMASHKDDEDKFFPMS |
| | | GVMIFGKESAGASNTALDNVMITDEEEIKATNPVA |
| | | TERFGTVAVNFQSSSGNNTRSVTDPATGDVHAMGA |
| | | LPGMVWQDRDVYLQGPIWAKIPHTDGHFHPSPLMG |
| | | GFGLKNPPPQILIKNTPVPANPPAEFSATKFASFI |
| | | TQYSTGQVSVEIEWELQKENSKRWNPEVQYTSNYA |
| | | KSANVDFTVDNNGLYTEPRPIGTRYLTRPL |
| X2 | 11 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTINGSG |
| | | QNQQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQGNNTRDT |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |
| X3 | 12 | MAADGYLPDWLEDNLSEGIREWWALKPGAPQPKAN |
| | | QQHQDNARGLVLPGYKYLGPGNGLDKGEPVNAADA |
| | | AALEHDKAYDQQLKAGDNPYLKYNHADAEFQERLK |
| | | EDTSFGGNLGRAVFQAKKRLLEPLGLVEEAAKTAP |
| | | GKKRPVEQSPQEPDSSAGIGKSGAQPAKKRLNFGQ |
| | | TGDTESVPDPQPIGEPPAAPSGVGSLTMASGGGAP |
| | | VADNNEGADGVGSSSGNWHCDSQWLGDRVITTSTR |
| | | TWALPTYNNHLYKQISNSTSGGSSNDNAYFGYSTP |
| | | WGYFDFNRFHCHFSPRDWQRLINNNWGFRPKRLNF |
| | | KLFNIQVKEVTDNNGVKTIANNLTSTVQVFTDSDY |
| | | QLPYVLGSAHEGCLPPFPADVFMIPQYGYLTLNDG |
| | | SQAVGRSSFYCLEYFPSQMLRTGNNFQFSYEFENV |
| | | PFHSSYAHSQSLDRLMNPLIDQYLYYLSRTINGSG |
| | | QNQQTLKFSVAGPSNMAVQGRNYIPGPSYRQQRVS |
| | | TTVTQNNNSEFAWPGASSWALNGRNSLMNPGPAMA |
| | | SHKEGEDRFFPLSGSLIFGKQGTGRDNVDADKVMI |
| | | TNEEEIKTTNPVATESYGQVATNHQSAQTNSTRPV |
| | | AQAQTGWVQNQGILPGMVWQDRDVYLQGPIWAKIP |
| | | HTDGNFHPSPLMGGFGMKHPPPQILIKNTPVPADP |
| | | PTAFNKDKLNSFITQYSTGQVSVEIEWELQKENSK |
| | | RWNPEIQYTSNYYKSNNVEFAVNTEGVYSEPRPIG |
| | | TRYLTRNL |

Sequences reported in this table are of AAV VP1 CAP proteins.

TABLE 2
7-mer insertions and substitutions

| Name | SEQ ID NO | Sequence |
|---|---|---|
| X1-7mer | 13 | GNNTRSV |
| X2-7mer | 14 | GNNTRDT |
| X3-7mer | 15 | TNSTRPV |
| X1.1-7mer | 16 | DGAATKN |
| X1.2-7mer | 17 | DGQSSKS |

X1-7mer, X2-7mer, and X3-7mer sequences are inserted between AA positions 588 and 589 of AAV capsid VP1 protein. X1.1-7mer, X1.2-7mer, and X1.3-7mer sequences are substituted into positions 452-458 of AAV capsid VP1 protein.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An adeno-associated virus (AAV) targeting peptide comprising an amino acid sequence that comprises at least 4 contiguous amino acids from a sequence selected from the group consisting of GNNTRSV (SEQ ID NO: 13), GNNTRDT (SEQ ID NO: 14) and TNSTRPV (SEQ ID NO: 15).

2. The AAV targeting peptide of claim 1, wherein the targeting peptide comprises at least 5 contiguous amino acids from the sequence of GNNTRSV (SEQ ID NO: 13).

3. The AAV targeting peptide of claim 1, wherein the targeting peptide comprises at least 6 contiguous amino acids from the sequence of GNNTRSV (SEQ ID NO: 13).

4. The AAV targeting peptide of claim 1, wherein the targeting peptide comprises GNNTRSV (SEQ ID NO: 13).

5. The AAV targeting peptide of claim 1, wherein the targeting peptide comprises at least 4 contiguous amino acids from the sequence GNNTRDT (SEQ ID NO: 14).

6. The AAV targeting peptide of claim 5, wherein the targeting peptide comprises at least 5 contiguous amino acids from the sequence of GNNTRDT (SEQ ID NO: 14).

7. The AAV targeting peptide of claim 5, wherein the targeting peptide comprises at least 6 contiguous amino acids from the sequence of GNNTRDT (SEQ ID NO: 14).

8. The AAV targeting peptide of claim 5, wherein the targeting peptide comprises GNNTRDT (SEQ ID NO: 14).

9. The AAV targeting peptide of claim 1, wherein the targeting peptide comprises at least 4 contiguous amino acids from the sequence TNSTRPV (SEQ ID NO: 15).

10. The AAV targeting peptide of claim 9, wherein the targeting peptide comprises at least 5 contiguous amino acids from the sequence of TNSTRPV (SEQ ID NO: 15).

11. The AAV targeting peptide of claim 9, wherein the targeting peptide comprises at least 6 contiguous amino acids from the sequence of TNSTRPV (SEQ ID NO: 15).

12. The AAV targeting peptide of claim 9, wherein the targeting peptide comprises TNSTRPV (SEQ ID NO: 15).

13. The AAV targeting peptide of any one of claims 1-12, wherein the targeting AAV peptide is part of an AAV.

14. The AAV targeting peptide of claim 13, wherein the targeting peptide is part of a capsid protein of the AAV.

15. The AAV targeting peptide of any one of claims 1-14, wherein the targeting peptide is conjugated to a nanoparticle, a second molecule, a viral capsid protein, or a combination thereof. [symbol]
peptide is a central nervous system (CNS) targeting peptide.

17. An adeno-associated virus (AAV) capsid protein comprising an AAV targeting peptide of any one of claims 1-16.

18. The AAV capsid protein of claim 17, further comprising at least 4 contiguous amino acids from a second amino acid sequence selected from the group consisting of DGQSSKS (SEQ ID NO: 17), DGAATKN (SEQ ID NO: 16), and LQTSSPG (SEQ ID NO: 18).

19. The AAV capsid protein of claim 17, further comprising at least 5 contiguous amino acids from a second amino acid sequence selected from the group consisting of DGQSSKS (SEQ ID NO: 17), DGAATKN (SEQ ID NO: 16), and LQTSSPG (SEQ ID NO: 18).

20. The AAV capsid protein of claim 17, further comprising at least 6 contiguous amino acids from a second amino acid sequence selected from the group consisting of DGQSSKS (SEQ ID NO: 17), DGAATKN (SEQ ID NO: 16), and LQTSSPG (SEQ ID NO: 18).

21. The AAV capsid protein of claim 17, further comprising a second amino acid sequence selected from the group consisting of DGQSSKS (SEQ ID NO: 17), DGAATKN (SEQ ID NO: 16), and LQTSSPG (SEQ ID NO: 18).

22. The AAV capsid protein of any one of claims 18-21, wherein the at least 4, 5 or 6 contiguous amino acids from the second amino acid sequence replace at least 4, 5, 6 or 7 amino acids in AA452-458, or functional equivalents thereof, of the AAV capsid protein.

23. The AAV capsid protein of claim 22, wherein the at least 4, 5 or 6 contiguous amino acids from the second amino acid sequence, or the second amino acid sequence, replace at least 4, 5, 6 or 7 amino acids in the 455 loop, or functional equivalents thereof, of the AAV capsid protein.

24. The AAV capsid protein of any one of claims 17-23, further comprising one or more of amino acid substitutions at position N272, S386, and W503.

25. The AAV capsid protein of any one of claims 17-23, further comprising one or more of amino acid substitutions N272A, S386A, W503A, and W503R.

26. The AAV capsid protein of any one of claims 17-25, wherein the AAV capsid is derived from AAV9, or a variant thereof.

27. The AAV capsid protein of any one of claims 17-25, wherein the AAV capsid is derived from an AAV selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10.

28. A nucleic acid, comprising a sequence encoding the AAV targeting peptide of any one of claims 1-16. [symbol]
one of claims 17-27.

30. A recombinant adeno-associated virus (rAAV), comprising AAV targeting peptide of any one of claims 1-16, or an AAV capsid protein of any one of claims 17-27.

31. A recombinant adeno-associated virus (rAAV), comprising an AAV capsid protein which comprises the AAV targeting peptide of any one of claims 1-16, wherein the amino acid sequence is inserted between two adjacent amino acids in AA586-592, or functional equivalents thereof, of the AAV capsid protein.

32. The rAAV of claim 31, wherein the two adjacent amino acids are AA588 and AA589.

33. The rAAV of any one of claims 30-32, wherein the AAV capsid protein comprises, or consists thereof, SEQ ID NOs: 1 or 2.

34. The rAAV of any one of claims 30-33, further comprising at least 4 contiguous amino acids from a second amino acid sequence selected from the group consisting of DGQSSKS (SEQ ID NO: 17), DGAATKN (SEQ ID NO: 16), and LQTSSPG (SEQ ID NO: 18).

35. The rAAV of any one of claims 30-33, further comprising at least 5 contiguous amino acids from a second amino acid sequence selected from the group consisting of DGQSSKS (SEQ ID NO: 17), DGAATKN (SEQ ID NO: 16), and LQTSSPG (SEQ ID NO: 18).

36. The rAAV of any one of claims 30-33, further comprising at least 6 contiguous amino acids from a second amino acid sequence selected from the group consisting of DGQSSKS (SEQ ID NO: 17), DGAATKN (SEQ ID NO: 16), and LQTSSPG (SEQ ID NO: 18).

37. The rAAV of any one of claims 30-33, further comprising a second amino acid sequence selected from the group consisting of DGQSSKS (SEQ ID NO: 17), DGAATKN (SEQ ID NO: 16), and LQTSSPG (SEQ ID NO: 18).

38. The rAAV of any one of claims 34-37, wherein the at least 4, 5, or 6 contiguous amino acids from a second amino acid sequence, or the second amino acid sequence, replace at least 4, 5, 6 or 7 amino acids in AA452-458, or functional equivalents thereof, of the AAV capsid protein.

39. The rAAV of any one of claims 34-37, wherein the at least 4, 5, or 6 contiguous amino acids from a second amino acid sequence, or the second amino acid sequence, replace at least 4, 5, 6 or 7 amino acids in the 455 loop, or functional equivalents thereof, of the AAV capsid protein.

40. The rAAV of any one of claims 30-39, further comprising one or more of amino acid substitutions at position N272, S386, and W503. [symbol]
acid substitutions N272A, S386A, W503A, and W503R.

42. The rAAV of any one of claims 30-41, wherein the rAAV comprises an rAAV vector genome.

43. The rAAV of claim 42, wherein the rAAV vector genome comprises one or more miRNA-122 (miR-122) binding sites.

44. The rAAV of claim 43, wherein the one or more miR-122 binding sites are located in the 3′ UTR of the rAAV vector genome.

45. A composition, comprising
an AAV targeting peptide of any one of claims 1-16,
an AAV capsid protein of any one of claims 17-27,
a nucleic acid of any one of claims 28-29,
an rAAV of any one of claims 30-44, or a combination thereof.

46. The composition of claim 45, wherein the composition is a pharmaceutical composition comprising one or more pharmaceutical acceptable carriers.

47. A composition for use in the delivery of an agent to a target environment of a subject in need, comprising an AAV comprising (1) an AAV capsid protein of any one of claims 17-27 and (2) an agent to be delivered to the target environment of the subject, wherein the target environment is the nervous system.

48. The composition for use of claim 47, wherein the target environment is the central nervous system (CNS), the peripheral nervous system (PNS), or a combination thereof.

49. The composition for use of any one of claims 47-48, wherein the target environment is brain endothelial cells, neurons, capillaries in the brain, arterioles in the brain, arteries in the brain, or a combination thereof.

50. The composition for use of any one of claims 47-49, wherein the composition is a pharmaceutical composition comprising one or more pharmaceutical acceptable carriers.

51. The composition for use of any one of claims 47-50, wherein the agent to be delivered comprises a nucleic acid, a peptide, a small molecule, an aptamer, or a combination thereof.

52. The composition for use of claim 51, wherein the nucleic acid comprises one or more of:
a) a DNA sequence that encodes a trophic factor, a growth factor, or a soluble protein;
b) a cDNA that restores protein function to humans or animals harboring a genetic mutation(s) in that gene; [symbol]
or state of a cell;
d) a cDNA that encodes a protein or a nucleic acid that can be used for assessing the state of a cell;
e) a cDNA that encodes a protein for gene editing, or a guide RNA;
f) a DNA sequence for genome editing via homologous recombination;
g) a DNA sequence encoding a therapeutic RNA;
h) an shRNA or an artificial miRNA delivery system; and
i) a DNA sequence that influences the splicing of an endogenous gene.

53. The composition for use of any one of claims 47-52, wherein the subject in need is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS.

54. The composition for use of claim 53, wherein the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

55. The composition for use of any one of claims 47-52, wherein the subject in need is a subject suffering from, at risk to develop, or has suffered from a stroke, traumatic brain injury, epilepsy, or spinal cord injury.

56. The composition for use of any one of claims 47-55, wherein the composition is for intravenous administration.

57. The composition for use of any one of claims 47-55, wherein the composition is for systemic administration.

58. The composition for use of any one of claims 47-57, wherein the agent is delivered to endothelial lining of the ventricles in the brain, central canal of the spinal cord, capillaries in the brain, arterioles in the brain, arteries in the brain, or a combination thereof of the subject.

59. The composition for use of any one of claims 47-58, wherein the subject is an adult animal.

60. A method of delivering an agent to a nervous system of a subject, the method comprising: [symbol]
claims 17-27, wherein the AAV vector further comprises an agent to be delivered to the nervous system; and
administering the AAV vector to the subject.

61. The method of claim 60, wherein the administration is a systemic administration.

62. The method of claim 60, wherein the administration is an intravenous administration.

63. The method of any one of claims 60-62, wherein the subject is a primate and the agent is delivered to the endothelial cells and neurons of the nervous system.

64. The method of any one of claims 60-63, wherein the agent is delivered to the endothelial cells of the nervous system of the subject at least 1.5-fold, 2-fold, or 3-fold more efficiently than the delivery of the agent to the neurons of the nervous system.

65. The method of any one of claims 60-64, wherein the nervous system is the central nervous system (CNS).

66. A method of delivering an agent to a cell, the method comprising: contacting an AAV vector comprising an AAV capsid protein of any one of claims 17-27 with the cell, wherein the AAV vector further comprises an agent to be delivered to the nervous system, and wherein the cell is an endothelial cell or a neuron.

67. The method of claim 66, wherein contacting the AAV vector with the cell occurs in vitro, in vivo or ex vivo.

68. The method of any one of claims 66-67, wherein the cell is present in a tissue, an organ, or a subject.

69. The method of any one of claims 66-68, wherein the cell is a brain endothelial cell, a neuron, a cell in the capillaries in the brain, a cell in the arterioles of the brain, a cell in the arteries in the brain, a cell in the brain vasculature, or a combination thereof.

70. The method of any one of claims 60-69, wherein the agent to be delivered comprises a nucleic acid, a peptide, a small molecule, an aptamer or a combination thereof.

71. The method of claim 70, wherein the nucleic acid encodes a therapeutic protein.

72. The method of claim 70, wherein the nucleic acid comprises one or more of:
a) a DNA sequence that encodes a trophic factor, a growth factor, or other soluble factors capable of being released from the transduced cells and affect the survival or function of that cell and/or surrounding cells;
b) a cDNA that restores protein function to humans or animals harboring a genetic mutation(s) in that gene; [symbol]
or state of a cell;
d) a cDNA that encodes a protein or a nucleic acid that can be used for assessing the state of a cell;
e) a cDNA that encodes a protein for gene editing, or a guide RNA;
f) a DNA sequence for genome editing via homologous recombination;
g) a DNA sequence encoding a therapeutic RNA;
h) an shRNA or an artificial miRNA delivery system; and
i) a DNA sequence that influences the splicing of an endogenous gene.

73. The method of any one of claims 60-72, wherein the AAV vector is an AAV9 vector, or a variant thereof.

74. The method of any one of claims 60-72, wherein the AAV vector is a vector selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof.

75. The method of any one of any one of claims 60-74, wherein the serotype of the AAV vector is different from the serotype of the AAV capsid.

76. The method of any one of claims 70-75, wherein the nucleic acid comprises one or more miRNA-122 (miR-122) binding sites.

77. The method of claim 76, wherein at least one of the one or more miR-122 binding sites is located in the 3′ UTR of the nucleic acid.
